(19) United States
(12) Patent Application Publication
(10) Pub. No.: US 2013/0109854 A1
(43) Pub. Date: May 2, 2013

(54) Novel Methods for Preparation of (+)-1-ethyl-4-[2-(4-morpholinyl)ethyl)-3,3-diphenyl-2-pyrrolidinone and Salts Thereof

(71) Applicants: Scott L. Dax, Landenberg, PA (US); Vita Ozola, Latvia (LV); Andrejs Krasikovs, Latvia (LV); Edgars Suna, Latvia (LV)

(72) Inventors: Scott L. Dax, Landenberg, PA (US); Vita Ozola, Latvia (LV); Andrejs Krasikovs, Latvia (LV); Edgars Suna, Latvia (LV)

(73) Assignee: Galleon Pharmaceuticals, Inc.

(21) Appl. No.: 13/451,060

(22) Filed: Apr. 19, 2012

Related U.S. Application Data
(63) Continuation-in-part of application No. 13/286,823, filed on Nov. 1, 2011.

(51) Int. Cl. C07D 413/06; C07D 207/08; C07D 207/26; C07D 207/09

(52) U.S. Cl. 544/141; 548/566; 548/568; 548/572; 548/543

(57) ABSTRACT

The present invention includes a method of preparing a composition comprising (+)-doxapram or a salt thereof, wherein the composition is essentially free of (−)-doxapram or a salt thereof.

### CROSS-REFERENCE TO RELATED APPLICATIONS

The present application is a continuation-in-part of and claims priority to, U.S. application Ser. No. 13/286,823, filed Nov. 1, 2011, which application is incorporated by reference herein in its entire.

### BACKGROUND OF THE INVENTION

Normal control of breathing is a complex process that involves the body's interpretation and response to chemical stimuli such as carbon dioxide, pH and oxygen levels in blood, tissues and the brain. Breathing control is also affected by wakefulness whether the patient is awake or sleeping). Within the brain medulla, there is a respiratory control center that interprets the various signals that affect respiration and issues commands to the muscles that perform the work of breathing. Key muscle groups are located in the abdomen, diaphragm, pharynx and thorax. Sensors located centrally and peripherally then provide input to the brain's central respiration control areas that enables response to changing oxygen requirements. Normal respiratory rhythm is maintained primarily by the body's rapid response to changes in carbon dioxide levels (CO₂). Increased CO₂ levels signal the body to increase breathing rate and depth, resulting in higher oxygen levels and subsequent lower CO₂ levels. Conversely, low CO₂ levels can result in periods of apnea since there is no stimulation to breathe.

The ability of a mammal to breathe, and to modify breathing according to the amount of oxygen available and demands of the body, is essential for survival. There are many diseases in which loss of normal breathing rhythm is a primary or secondary feature of the disease. Examples of diseases with a primary loss of breathing rhythm control are apneas (central, mixed or obstructive; where the breathing repeatedly stops for 10 to 60 seconds) and congenital central hypoventilation syndrome. Secondary loss of breathing rhythm may be due to chronic cardio-pulmonary diseases (e.g., heart failure, chronic bronchitis, emphysema, and impending respiratory failure), excessive weight (e.g., obesity-hypoventilation syndrome), certain drugs (e.g., anesthetics, sedatives, anxiolytics, hypnotics, alcohol, and narcotic analgesics) and/or factors that affect the neurological system (e.g., stroke, tumor, trauma, radiation damage, and ALS). In chronic obstructive pulmonary diseases where the body is exposed to chronically low levels of oxygen, the body adapts to the lower pH by kidney-mediated retention of bicarbonate, which has the effect of partially neutralizing the CO₂/pH respiratory stimulation.

Sleep apnea is characterized by frequent periods of no or partial breathing, Key factors that contribute to these apneas include decrease in CO₂ receptor sensitivity, decrease in hypoxic ventilatory response sensitivity (e.g., decreased response to low oxygen levels) and loss of “wakefulness.” Apnea events result in hypoxia (and the associated oxidative stress) and eventually severe cardiovascular consequences (high blood pressure, stroke, heart attack). Snoring has some features in common with steep apnea, with the upper airway muscles losing their tone, resulting in snoring sounds and also inefficient airflow and hypoxia.

Racemic 1-ethyl-4-[2-(4-morpholinyl-ethyl)-3,3-diphenyl-2-pyrrolidinone (commonly known as doxapram) is a known respiratory stimulant, marketed under the name of Dopram™.

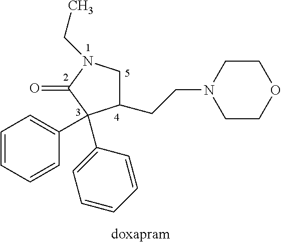 [SMILES: CCN1CC(CCN2CCOCC2)C(c2ccccc2)(c2ccccc2)C1=O]

Doxapram has a strong, dose-dependent effect on stimulating respiration (breathing) in animals (Ward & Frank®, 1962, Fed. Proc. 21:325), Administered intravenously, doxapram causes an increase in tidal volume and respiratory rate. Doxapram is used in intensive care settings to stimulate respiration in patients with respiratory failure and to suppress shivering after surgery. Doxapram is also useful for treating respiratory depression in patients who have taken excessive doses of drugs such buprenorphine and fail to respond adequately to treatment with naloxone. However, use of doxapram in the medical setting is hampered by side effects, such as high blood pressure, panic attacks, tachycardia (rapid heart rate), tremor, convulsions, sweating, vomiting and the sensation of “air hunger.” Doxapram may not be used in patients with coronary heart disease, epilepsy and high blood pressure.

The C-4 carbon in the structure of doxapram is a chiral center, and thus there are two distinct enantiomers associated with this molecule: the (+)-enantiomer and the (−)-enantiomer. The concept of enantiomers is well known to those skilled in the art. The two enantiomers have the same molecular formula and identical chemical connectivity, but opposite spatial “handedness,” being a mirror image of each other and not superimposable, Chiral molecules have the unique property of causing a rotation in the original plane of vibration of plane-polarized light. Individual enantiomers are able to rotate plane-polarized light in a clockwise (dextrorotary; the (+)-enantiomer) or counterclockwise (levorotatory; the (−)-enantiomer) manner. For a specific combination of solvent, concentration and temperature, the pure enantiomers rotate plane-polarized light by the same number of degrees but in opposite directions.

A racemic mixture or a “racemate” is a term used to indicate the mixture of essentially equal quantities of enantiomeric pairs. Racemic mixtures are devoid of appreciable optical activity due to the mutually opposing optical activities of the individual enantionters. Apart from their interaction with polarized light, enantiomers may differ in their physical, chemical and pharmacological activities, but such differences between enantiomers are largely unpredictable.

Enantiomers may be separated from a racemate, or synthesized directly using enantioselective methods. An enantiomer can be prepared from a chiral starting material or an intermediate containing the same stereochemistry of the desired enantiomeric product. Alternatively, since chiral centers may often be inverted, intermediates of opposite stereochemistry may also be used in such synthetic approaches. It may also be possible to develop and use an asymmetric synthetic route in which the desired stereochemistry of the enantiomer is installed through stereocontrolled bond formation of an achiral species, as opposed to the use of a chiral starting material. In this approach, the stereochemistry of the chiral center arises from a chiral auxiliary, such as a chiral catalyst, acid or base, that directs bond formation from one face (direction) within a homochiral environment. Such synthetic techniques need to be highly specific and typically must be optimized for any given enantiomer.

Chiral resolution involves the derivatization of a racemate [(+)-ent and (−)-ent] with optically pure reagents, also known as chiral partners (CP), to generate diastereomeric complexes or adducts (Scheme 1). The resultant diastereomeric mixture [(+)-ent•CP and (−)-ent•CP] may then be separated by crystallization techniques in the case of diastereomeric complexes, or by chromatography in the case of diastereomeric adducts. The selection of the chiral partnering agent is important as to provide diastereomeric complexes or adducts that may be separated by crystallization or chromatography. After separation, the chiral partner may be removed, or the adduct with the chiral partnering agent may be dissociated, and the respective enantiomers may be obtained in optically-enriched or optically-pure form,

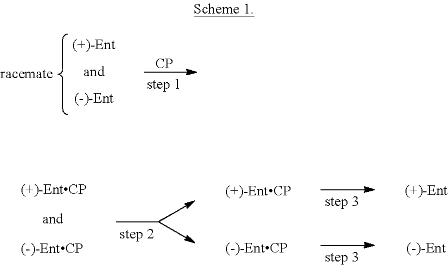

Chiral resolution and chiral chromatography techniques are exemplified in Schemes 2-4, wherein each occurrence of R* is independently a chiral group. In Scheme 2, a racemic amine (R*—NR2) may be treated with a chiral organic acid (such as a tartaric acid derivative; depicted as R*—COOH) to produce the corresponding diastereomeric ammonium salts. Crystallization effects separation of the diastereomeric pair. Removal of the chiral organic acid serving as the chiral partnering agent, by neutralization and extraction for example, affords pure enantiomer(s).

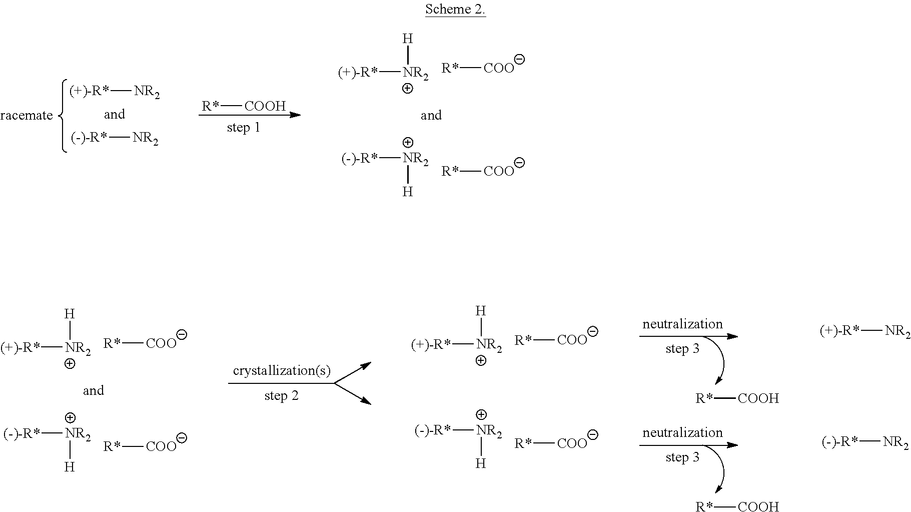

As illustrated in Scheme 3, a racemic carboxylic acid (R*—COOH) may treated with a chiral organic alcohol (R*—OH) (such as 1-phenylethanol for example) to produce the corresponding diastereomeric esters. Chromatography effects separation of the diastereomeric pair. Cleavage of the ester, by acid- or base-mediated hydrolysis for example, affords pure enantiomer(s).

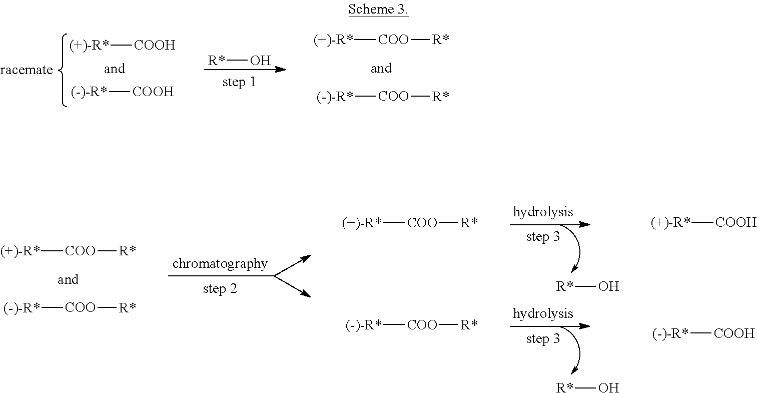

Alternatively, racemates may be directly physically separated into respectively enantiomers via chiral chromatography (Scheme 4). This technique involves contacting a solution of the racemate with a surface containing an immobilized chiral partner, referred to as a chiral stationary phase (CSP). The association of each individual enantiomer with the chiral partner (CSP) effects separation since one enantiomer has favorable interactions with the CSP, whereas the opposite enantiomer has disfavored or less favored interactions with the CSP.

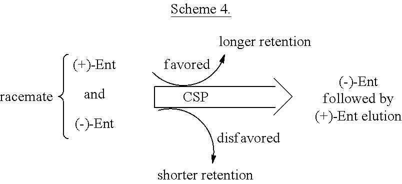

Recent attempts have been made to develop pure enantiomers as new drugs, based on previously marketed racemic drugs (Nunez et al., 2009, Curr. Med. Chem. 16(16):2064-74), Development of an individual enantiomer as a novel drug, based on the already used racemate, requires separation of the racemate into its enantiomeric components (or diastereomeric components, if the molecule has more than one chiral center and the de novo pharmacokinetic, pharmacological and toxicological characterization of each enantiomer (or diastereomer), since its properties may differ substantially and unpredictably from those of the racemate.

Doxapram is marketed and medically used as a racemate. Doxapram has been previously separated into its pure enantiomers using methods such as chiral high-performance liquid chromatography (Chankvetadze et al., 1996, J. Pharm. Biomed. Anal. 14:1295-1303; Thunberg et al., 2002, J. Pharm. Biomed. Anal, 27:431-39), and chiral capillary electrophoresis (Christians & Holzgrabe, 2001, J. Chromat. A 911:249-57). Using in silico methods, the enantiomers of doxapram were predicted to have identical oral bioavailability (Moda et al., 2007, Bioorg. Med. Chem, 15:7738-45).

There is a need in the art for the identification of a compound that may be used to treat breathing control disorders or diseases. Such a compound should restore all or part of the body's normal breathing control system in response to changes in CO₂ and/or oxygen, and yet have minimal side effects. There is also a need in the art for the identification of a synthetic route that allows for the large-scale synthesis of the compound with reliably high yield, enantioselectivity and purity. The present invention fulfills these needs.

### BRIEF DESCRIPTION OF THE INVENTION

The invention includes a composition comprising at least one crystalline salt of (R)-doxapram selected from the group consisting of:

(i) a crystalline sulfate salt of (R)-doxapram, wherein the XRPD spectrum of the salt comprises 28 values (in) of 7.97±0.20, 8.56±0.20, 10.40±0.20, 11.00±0.20, 12.48±0.20, 15.69±0.20, 17.16±0.20, 17.98±0.20, 18.67±0.20, 19.69±0.20, 20.17±0.20, 20.89±0.20, 21.12±0.20, 21.66±0.20, 22.23±0.20, 23.21±0.20, 23.53±0.20, 24.15±0.20, 24.62±0.20, 25.94±0.20, and 27.09±0.20;

(ii) a crystalline 1)-glucuronate salt of (R)-doxapram, wherein the XRPD spectrum of the salt comprises 2θ values (in°) of 7.64±0.20, 8.69±0.20, 9.76±0.20, 11.63±0.20, 12.33±0.20, 13.34±0.20, 15.33±0.20, 15.98±0.20, 16.96±0.20, 17.43±0.20, 18.08±0.20, 18.55±0.20, 19.54±0.20, 20.21±0.20, 20.61±0.20, 20.91±0.20, 21.56±0.20, 22.73±0.20, 23.35±0.20, 24.82±0.20, and 25.37±0.20;

(iii) a crystalline maleate salt of (R)-doxapram, wherein the XRPD spectrum of the salt comprises 2θ values (in°) of (5.88-5.93)±0.20, (9.56-9.58)±0.20, (12.43-12.57)±0.20, (13.45±13.49)±0.20, (15.23-15.29)±0.20, (19.24-19.25)±0.20, (19.60-19.79)±0.20, (20.06-20.07)±0.20, (20.52-20.54)±0.20, 21.21-21.29)±0.20, (22.65-22.66)±0.20, and (23.00-23.05)±0.20;

(iv) a crystalline oxalate salt of (R)-doxapram, wherein the XRPD spectrum of the salt comprises 2θ values (in°) of 8.64±0.20, 9.93±0.20, 11.03±0.20, 15.89±0.20, 17.33±0.20, 17.62±0.20, 17.92±0.20, 18.30±0.20, 18.72±0.20, 19.62±0.20, 19.87±0.20, 20.94±0.20, 23.20±0.20, 23.53±0.20, and 24.00±0.20;

(v) a crystalline L-tartrate salt of (R)-doxapram, wherein the XRPD spectrum of the salt comprises 2θ values (in°) of (5.90-5.95)±0.20, (8.49-8.54)±0.20, (10.03-10.10)±0.20, (10.68-10.70)±0.20, (12.72-12.80)±0.20, (14.67-14.77)±0.20, (15.04-15.13)±0.20, (18.43-18.57)±0.20, (20.09-20.17)±0.20, and (20.67-20.79)±0.20;

(vi) a crystalline phosphate salt of (R)-doxapram, wherein the XRPD spectrum of the salt comprises 2θ values (in c) of 9.56±0.20, 10.76±0.20, 11.83±0.20, 13.47±0.20, 13.92±0.20, 15.33±0.20, 17.57±0.20, 18.74±0.20, 19.17±0.20, 20.07±0.20, 21.28±0.20, 22.16±0.20, 21.66±0.20, 22.46+0.20, and 28.13±0.20;

(vii) a crystalline phosphate salt of (R)-doxapram, wherein the XRPD spectrum of the salt comprises 2θ values (in c) of 9.63±0.20, 9.73±0.20, 10.48±0.20, 11.95±0.20, 11.95±0.20, 13.37±0.20, 13.65±0.20, 15.21±0.20, 17.43±0.20, 18.45±0.20, 19.02±0.20, 20.31±0.20, 21.04±0.20, 21.99±0.20, 22.38±0.20, 23.46±0.20, and 23.93±0.20;

(viii) a crystalline hydrochloride salt of (R)-doxapram, wherein the XRPD spectrum of the salt comprises 2θ values (in°) of 7.47±0.20, 10.12±0.20, 11.39+0.20, 11.96±0.20, 12.64±0.20, 14.68±0.20, 15.87±0.20, 16.88±0.20, 17.44±0.20, 17.83±0.20, 18.40±0.20, 10.24±0.20, 19.76±0.20, 20.72±0.20, 21.68±0.20, 22.8.4±0.20, 23.40±0.20, 24.36+0.20, 25.44+0.20, 26.24+0.20, 27.07+0.20, 28.13+0.20, 29.16±0.20, 29.88±0.20, 30, 83, 31.20±0.20; and any combinations thereof.

In one embodiment, the salt in (i) comprises about one molar equivalent of sulfuric acid. In another embodiment, the salt in (ii) comprises about one molar equivalent of D-glucuronic acid. In yet another embodiment, the salt in (iii) comprises about one molar equivalent of maleic acid. In yet another embodiment, the salt in (iv) comprises about one molar equivalent of oxalic acid. In yet another embodiment, the salt in (v) comprises about one molar equivalent of L-tartaric acid and about 0.4 to 0.6 molar equivalents of methanol. In yet another embodiment, the salt in (vi) comprises about one molar equivalent of phosphoric acid and about one molar equivalent of water. In yet another embodiment, the salt in (vii) comprises about one molar equivalent of phosphoric acid and about 0.6 molar equivalents of water. In yet another embodiment, the salt in (viii) comprises one molar equivalent of hydrochloric acid and about one molar equivalent of water.

The invention further includes a composition comprising at least one compound selected from the group consisting of: (R)-2-(1-Ethylpyrrolidin-3-yl)-2,2-diphenylacetonitrille; (R)-2-(1-Ethylpyrrolidin-3-yl)-2,2-diphenylacetamide; (R)-2-(1-Ethylpyrrolidin-3-yl)-2,2-diphenylacetic acid; (R)-1-Ethyl-4-(2-iodo-ethyl)-3,3-diphenyl-pyrrolidin-2-one; (R)-1-Ethyl-4-(2-d₈-morpholin-4-yl-ethyl)-3,3-diphenyl-pyrrolidin-2-one; a salt thereof, and any combinations thereof.

The invention further includes a method of preparing a composition comprising doxapram or a salt thereof. The method comprises cyclizing an ester derivative of (Z)-4-diphenylacetyl-ethyl-amino)-but-2-en-1-ol to generate 1-ethyl-3,3-diphenyl-4-vinyl-pyrrolin-2-one. The method further comprises derivatizing diphenyl-4-vinyl-pyrrolin-2-one to generate 4-(2-X-ethyl)-1-ethyl-3,3-diphenyl-pyrrolidin-2-one, wherein X is a leaving group. The method further comprises reacting 4-(2-X-ethyl)-1-ethyl-3,3-diphenyl-pyrrolidin-2-one with morpholine to generate doxapram or a salt thereof.

In one embodiment, the ester derivative is acetate. In another embodiment, X is selected from the group consisting of fluoride, chloride, bromide, triflate, tosylate and mesylate. In yet another embodiment, the cyclizing comprises reacting the ester derivative of (Z)-4-diphenylacetyl-ethyl-amino)-but-2-en-1-ol with a strong base. In yet another embodiment, the strong base comprises potassium hexamethyldisilazide, sodium hexamethyldisilazide or sparteine. In yet another embodiment, the cyclizing comprises the steps of: derivatizing the ester derivative of (Z)-4-diphenylacetyl-ethyl-amino)-but-2-en-1-ol to generate a carbonate derivative of (g)-4-diphenylacetyl-ethyl-amino)-but-2-en-1-ol; and, reacting the carbonate derivative of (Z)-4-diphenylacetyl-ethyl-amino)-but-2-en-1-ol with a strong base to generate 1-ethyl-3,3-diphenyl-4-vinyl-pyrrolin-2-one. In yet another embodiment, the strong base comprises potassium hexamethyldisilazide, sodium hexamethyldisilazide or sparteine. In yet another embodiment, the doxapram or a salt thereof is enantiomerically enriched in (R)-doxapram. In yet another embodiment, (R)-doxapram or a salt thereof is in at least about 70% enantiomeric excess in the composition. In yet another embodiment, the cyclizing comprises the steps of: hydrolyzing the ester derivative of (Z)-4-diphenylacetyl-ethyl-amino)-but-2-en-1-ol to generate (Z)-4-diphenylacetyl-ethyl-amino)-but-2-en-1-ol; derivatizing (Z)-4-diphenylacetyl-ethyl-amino)-but-2-en-1-ol to generate bis(η³-1-(ethyl-(diphenylacetyl)aminomethylyl)-allyl)-dichloro-dipalladium(II); and, reacting bis(η³-1-(ethyl-(diphenylacetyl)aminomethylyl)-allyl)-dichloro-dipalladium(II) with R-BINAP in the presence of a strong base to generate 1-ethyl-3,3-diphenyl-4-vinyl-pyrrolin-2-one enriched in the (R)-enantiomer.

The invention further includes a method of preparing a composition comprising (R)-doxapram, wherein in the composition (R)-doxapram is in an enantiomeric excess over (S)-doxapram. The method comprises hydrolyzing (R)-2-(1-ethylpyrrolidin-3-yl)-2,2-diphenylacetonitrile to generate (R)-2-(1-ethylpyrrolidin-3-yl)-2,2-diphenylacetic acid. The method further comprises derivatizing (R)-2-(1-ethylpyrrolidin-3-yl)-2,2-diphenylacetic acid to generate (R)-1-ethyl-4-(2-iodoethyl)-3,3-diphenylpyrrolidin-2-one. The method further comprises reacting (R)-1-ethyl-4-(2-iodoethyl)-3,3-diphenylpyrrolidin-2-one with morpholine to generate (R)-doxapram.

In one embodiment, the hydrolyzing comprises hydrolyzing (R)-2-(1-ethylpyrrolidin-3-yl)-2,2-diphenylacetonitrile with acid to generate (R)-2-(1-ethylpyrrolidin-3-yl)-2,2-diphenylacetamide; and hydrolyzing (R)-2-(1-ethylpyrrolidin-3-yl)-2,2-diphenylacetamide with acid to generate (R)-2-(1-ethylpyrrolidin-3-yl)-2,2-diphenylacetic acid. In another embodiment, the derivatizing comprises contacting (R)-2-(1-ethylpyrrolidin-3-yl)-2,2-diphenylacetic acid with acetic anhydride and an iodide salt. In another embodiment, the (R)-2-(1-ethylpyrrolidin-3-yl)-2,2-diphenylacetonitrile to be used is of at least about 90% enantiomeric excess. In yet another embodiment, the (R)-2-(1-ethylpyrrolidin-3-yl)-2,2-diphenylacetonitrile to be used is of at least about 95% enantiomeric excess. In yet another embodiment, the (R)-2-(1-ethylpyrrolidin-3-yl)-2,2-diphenylacetonitrile to be used is of at least about 98% enantiomeric excess. In yet another embodiment, the (R)-2-(1-ethylpyrrolidin-3-yl)-2,2-diphenylacetonitrile to be used is of at least about 99% enantiomeric excess. In yet another embodiment, the (R)-2-(1-ethylpyrrolidin-3-yl)-2,2-diphenylacetonitrile to be used is chirally enriched by diastereoselective precipitation of its salt with a chiral organic acid. In yet another embodiment, the chiral organic acid comprises (+)-dibenzoyl-D-tartaric acid (D-DBTA). In yet another embodiment, (R)-2-(1-ethylpyrrolidin-3-yl)-2,2-diphenylacetonitrile is generated by contacting (R)-3-X-1-ethylpyrrolidine with a reaction mixture comprising diphenylacetonitrile and a strong base, wherein X is a leaving group. In yet another embodiment, X is selected from the group consisting of chloride, bromide, iodide, tosylate, mesylate and triflate. In yet another embodiment, X is chloride and the reaction mixture further comprises a promoter. In yet another embodiment, the promoter comprises an iodide salt and the contacting is performed at a temperature of about 50° C. to about 145° C. In yet another embodiment, (R)-3-chloride-1-ethylpyrrolidine is generated from (S)-1-ethylpyrrolidin-3-ol. In yet another embodiment, (S)-1-ethylpyrrolidin-3-ol is reacted with thionyl chloride to generate (R)-3-chloride-1-ethylpyrrolidine. In yet another embodiment, X is tosylate or mesylate. In yet another embodiment, the contacting is performed at a temperature of about 50° C. to about 145° C. In yet another embodiment, (R)-3-X-1-ethylpyrrolidine is generated by reacting (R)-1-ethyl-3-pyrrolidinol with tosyl chloride or mesyl chloride. In yet another embodiment, (R)-3-X-1-ethylpyrrolidine is not isolated before being derivatized to generate (R)-2-(1-ethylpyrrolidin-3-yl)-2,2-diphenylacetonitrile.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments that are presently preferred. It should be understood, however, that the invention is not limited to the precise arrangements and instrumentalities of the embodiments shown in the drawings.

FIG. 1 is a schematic illustration of a method implemented for the chiral separation of doxapram.

FIG. 2 is a graph illustrating the resolution of doxapram (labeled PCT 10052 racemate) using chiral chromatography. The enantiomers were detected in an enantiomeric ratio of 49.34 and 50.66% for Peak 1 and 2, respectively.

FIG. 3 is a graph illustrating a chiral chromatography analysis of (R)-doxapram (peak 2 in FIG. 2), which indicated an enantiomeric purity of 98.2%.

FIG. 4 is a graph illustrating a chiral chromatography analysis of (S)-doxapram (Peak 1 in FIG. 2), which indicated an enantiomeric purity of 99.9%.

FIG. 5 is an illustration of the ¹H NMR spectrum for the (+)-doxapram ((R)-doxapram) in CDCl₃ at 25° C.

FIG. 6 is an illustration of the ¹H NMR spectrum for the (+)-doxapram ((R)-doxapram) in CDCl₃ at 25° C.

FIG. 7 is an illustration of the ¹³C NMR spectrum for the (+)-doxapram ((R)-doxapram) CDCl₃ at 25° C.

FIG. 8 is an illustration of the ¹³C NMR spectrum for the (+)-doxapram ((R)-doxapram) CDCl₃ at 25° C.

FIG. 9 is an illustration of the IR spectrum of (R)-doxapram hydrochloride monohydrate.

FIG. 10 is an illustration of the thermal analysis profile by DSC for (R)-doxapram hydrochloride monohydrate.

FIG. 11 is art illustration of the X-ray powder diffraction profile for (R)-doxapram hydrochloride monohydrate.

FIG. 12, comprising FIGS. 12A-12C, is a series of graphs illustrating the vibrational circular dichroism analysis performed with the doxapram enantiomers.

FIG. 13, comprising FIGS. 13A-13C, illustrates calculated low-energy conformers of (R)-doxapram. FIGS. 13A and 13B illustrate optimized geometries of the four calculated lowest-energy conformers of the (R)-configuration. FIG. 13C is a series of graphs illustrating the VCD (upper frame) and IR (lower frame) spectra observed for (R)-doxapram (“Observed”) compared with calculated spectra for the eight calculated conformations for the (R)-configuration (“Calculated”).

FIG. 14 illustrates the VCD and IR spectra for (+)-doxapram (GAL-C054) and (−)-doxapram (GAL-C053). IR (lower frame) and VCD (upper frame) spectra of (+)-doxapram and (−)-doxapram CDCl₃ (5.9 mg/0.12 mL and 9.6 mg/0.2 mL); 100-μm path-length cell with BaF₂ windows; 7 h collection for both samples and solvent; instrument optimized at 1400 cm⁻¹. Solvent-subtracted IR and enantiomer-subtracted VCD spectra are shown. Uppermost traces are the VCD noise spectra.

FIG. 15 illustrates the IR and VCD Spectra of (R)-doxapram (GAL-C054). IR (lower frame) and VCD (upper frame) spectra of (R)-doxapram Lot # VGP-80 in DMSO-d₆ (9 mg/0.13 mL) and Lot #11-06507-6S (6 mg/0.12 mL) compared with those of Lot #11-06552-3S (8 mg/0.125 mL); 100-μL); 100-red with those of windows; 10 h collection for Lot # VGP-80, Lot #11-06507-6S, and DMSO-d₆, and 4 h collection for Lot #11-06552-3S; instrument optimized at 1400 cm⁻¹. Solvent-subtracted spectra are illustrated for the IR and VCD of all batches.

FIG. 16 illustrates a sample trace for chiral HPLC analysis of (R,S)-2-(1-ethylpyrrolidin-3-yl)-2,2-diphenylacetonitrile (19).

FIG. 17 illustrates a sample trace for chiral HPLC analysis of (R,S)-1-ethyl-4-(2-iodo-ethyl)-3,3-diphenyl-pyrrolidin-2-one (22).

FIG. 18 illustrates a sample trace for chiral HPLC analysis of (±)-doxapram.

FIG. 19 illustrates a XRPD diffractogram of the D-glucuronate salt of (+)-doxapram, (28). Peak labels in this image are meant as a visual aid. Table 20 lists accurate 2θ positions of prominent signals.

FIG. 20 illustrates a XRPD diffractogram of the sulfate salt of (+)-doxapram, (29). Peak labels in this image are meant as a visual aid. Table 21 lists accurate 2θ positions of prominent signals.

FIG. 21 and FIG. 22 illustrate XRPD diffractograms of the maleate salt of (+)-doxapram, (30). Peak labels in this image are meant as a visual aid. Table 22 lists accurate 2θ positions of prominent signals.

FIG. 23 illustrates a XRPD diffractogram of the oxalate salt of (+)-doxapram, (31). Peak labels in this image are meant as a visual aid. Table 23 lists accurate 2θ positions of prominent signals.

FIG. 24 and FIG. 25 illustrate XRPD diffractograms of the tartrate salt of (+)-doxapram, (32). Peak labels in this image are meant as a visual aid. Table 24 lists accurate 2θ positions of prominent signals.

FIG. 26 and FIG. 27 illustrate XRPD diffractograms of the phosphate salt of (+)-doxapram, (33—Form A; 34—Form B). Peak labels in this image are meant as a visual aid. Table 25 and Table 26 list accurate 2θ positions of prominent signals.

FIG. 28 illustrates an overlay of XRPD patterns of (+)-doxapram phosphate salts. From top to bottom: 33—Form A; 34—Form B, and 35—Form C.

### DETAILED DESCRIPTION OF THE INVENTION

The present invention includes a method of preparing the (+)-enantiomer of doxapram (Compound 1; known as (1)-doxapram, or (+)-4-(2-(1-ethyl-5-oxo-4,4-diphenylpyrrolidin-3-yl)ethyl)morpholine, or (+)-ethyl-4-[2-(4-morpholinyl)ethyl]-3,3 diphenyl-2-pyrrolidinone) or a salt thereof. In one embodiment, the (+)-doxapram is essentially free of (−)-doxapram or a salt thereof. In another embodiment, the method of preparing (+)-doxapram comprises an enantiomeric separation comprising formation of a diastereomeric salt and recrystallization of the salt. In yet another embodiment, the method of preparing (+)-doxapram comprises chiral chromatography separation of the enantionters of doxapram. In yet another embodiment, the method of preparing (+)-doxapram comprises an asymmetric synthesis starting from achiral starting materials. In yet another embodiment, the method of preparing (+)-doxapram comprises a synthesis starting from a chiral starting material or intermediate. In yet another embodiment, the salts of (+)-doxapram comprise specific crystalline polymorphic forms offering medicinally useful solubility properties and useful manufacturing properties. In one aspect, (+)-doxapram is (R)-doxapram, and (−)-doxapram is (S)-doxapram.

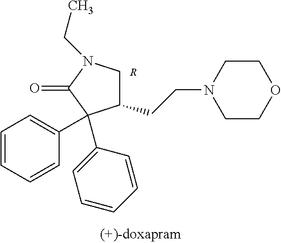

The present invention also includes a pharmaceutical formulation comprising the (+)-enantiomer of doxapram (known as (+)-doxapram, or (+)-4-(2-(1-ethyl-5-oxo-4,4-diphenylpyrrolidin-3-yl)ethyl)morpholine, or (+)-ethyl-4-[2-(4-morpholinyl)ethyl]-3,3-diphenyl-2-pyrrolidinone) or a salt thereof, and a pharmaceutically acceptable carrier, wherein the formulation is essentially free of (−)-doxapram or a salt thereof.

In one aspect, the present invention relates to the unexpected discovery that the (+)-enantiomer of doxapram displays most or all the desired beneficial pharmacological activity associated with the racemic doxapram. In another aspect, the present invention relates to the unexpected discovery that the (−)-enantiomer of doxapram is essentially devoid of activity in stimulating ventilation or reversing respiratory depression, and moreover produces a number of acute side effects in animals that were not detected as the same doses with (+)-doxapram, such as hunching posture, increased urination and defecation, clonic movements and other seizure-like behaviors, pronounced drops in mean arterial blood pressure, and production of cardiac arrhythmias and death.

A composition comprising (+)-doxapram or a salt thereof, wherein the composition is essentially free of (−)-doxapram or a salt thereof, may be administered to a subject who is prone to or suffers from a breathing control disorder or disease in order to prevent, treat or mitigate the breathing control disorder of a composition comprising (+)-doxapram or a salt thereof wherein the composition is essentially free of (−)-doxapram or a salt thereof is unexpectedly advantageous over administration of racemic doxapram or a salt thereof, because (+)-doxapram or a salt thereof has most or all the desired beneficial pharmacological respiratory stimulant activity, together with positive effects on arterial blood gases, associated with racemic doxapram but with significantly reduced adverse side effects compared to administration of racemic doxapram or a salt thereof.

The compositions of the invention are thus useful for treating a respiratory disease or disorder in a subject in need thereof. The respiratory disease or disorder includes, but is not limited to, respiratory depression (induced by anesthetics, sedatives, anxiolytic agents, hypnotic agents, alcohol, and analgesics), sleep apnea, apnea of prematurity, obesity-hypoventilation syndrome, primary alveolar hypoventilation syndrome, dyspnea, altitude sickness, hypoxia, hypercapnia and chronic obstructive pulmonary disease (COPD). The method comprises administering to the subject a therapeutically effective amount of a pharmaceutical formulation comprising a composition of the invention.

### DEFINITION

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, animal pharmacology, and organic chemistry are those well-known and commonly employed in the art.

As used herein, the articles “a” and “an” refer to one or to more than one (i.e. to at least one) of the grammatical object of the article. By way of example, “an element” means one element or more than one element.

A “subject”, as used therein, may be a human or non-human mammal. Non-human mammals include, for example, livestock and pets, such as ovine, bovine, porcine, canine, feline and murine mammals. Preferably, the subject is human.

As used herein, the term “about” will be understood by persons of ordinary skill in the art and will vary to some extent on the context in which it is used.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “isolated” as applied to a material refers to a material that has been partially, substantially or completely separated from the reaction mixture in which it may be formed, or from the solvent(s) into which it may be extracted, to generate a solution, slurry or solid that is enriched in the material.

As used herein, the term “IPC” refers to In-Process Controls. IPCs are assays conducted during the course of the process to monitor progress (e.g., pH, and absence of starting material) and/or to evaluate quality of material (e.g., purity, and enantiomeric excess).

As used herein, the term “L-DBTA” refers to dibenzoyl-L-tartaric acid or a salt thereof.

As used herein, the term “D-DBTA” refers to dibenzoyl-D-tartaric acid or a salt thereof.

As used herein, the term “CSP” refers to a chiral stationary phase.

As used herein, the term “doxapram” refers to 4-(2-(1-ethyl-5-oxo-4,4-diphenylpyrrolidin-3-yl)ethyl)morpholine, or ethyl-4-[2-(4-morpholinyl)ethyl]-3,3-diphenyl-2-pyrrolidinone), or a salt thereof. Unless otherwise noted, “doxapram” refers to racemic doxapram, which comprises an essentially equimolar mixture of the two enantiomers doxapram (the (+)-enantiomer and the (−)-enantiomer).

As used herein, the “(+)-doxapram” and “(−)-doxapram” enantiomers are defined in terms of the order in which they are eluted from chiral HPLC column, defined as: (a) a CHIRALPAK® AY 20μ column, with 3 cm internal diameter×25 cm length, using ethanol with 0.2% DMEA (dimethylethylamine) and CO₂ as mobile phase, in a ratio of 15:85, with a flow rate of 85 g/min, a column temperature of 35° C., and UV detection at 220 nm; or (b) a CHTRALPAK® AY-H 5μ column, with 3 cm internal diameter×25 cm length, using ethanol with 0.2% DMEA and CO₂ as mobile phase, in a ratio of 15:85, with a flow rate of 85 g/min, a column temperature of 35° C., and UV detection at 220 nm. Under either condition, the (−)-doxapram enantiomer has a shorter elution/retention time from the column than the (+)-doxapram enantiomer. The nomenclature “(+)-doxapram” should not be construed to imply that this enantiomer rotates the vibrational plane of plane-polarized light in a clockwise manner under all possible combinations of solvent, temperature and concentration. Similarly, the nomenclature “(−)-doxapram” should not be construed to imply that this enantiomer rotates the vibrational plane of plane-polarized light in a counter-clockwise manner under all possible combinations of solvent, temperature and concentration.

In a non-limiting embodiment, the absolute stereochemistry of (+)-doxapram and (−)-doxapram enantiomers may be defined by comparing actual vibrational circular dichroism (VCD) spectra to calculated VCD spectra. In this technique, an experimentally obtained VCD spectrum is overlaid upon a computationally generated VCD spectra generated from low-energy conformers using ab initio techniques and absolute stereochemistry, with probability values, and assigned based upon amplitude direction and absorption peak values. Using these techniques, the (+)-enantiomer of doxapram is assigned the (R)-stereochemical configuration, and the H-enantiomer of doxapram is assigned the (S)-stereochemical configuration.

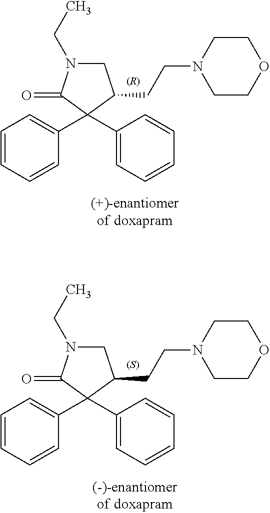

As used herein, the term “enantiomeric purity” of a given enantiomer over the opposite enantiomer indicates the excess % of the given enantiomer over the opposite enantiomer, by mole. For example, in a mixture comprising about 80% of a given enantiomer and about 20% of the opposite enantiomer, the enantiomeric purity of the given enantiomer is about 60%.

As used herein, the term “essentially free or” as applied to a given enantiomer in a mixture with the opposite enantiomer indicates that the enantiomeric purity of the given enantiomer is higher than about 80%, more preferably higher than about 90%, even more preferably higher than about 95%, even more preferably higher than about 97%, even more preferably higher than about 99%, even more preferably higher than about 99.5%, even more preferably higher than about 99.9%, even more preferably higher than about 99.95%, even more preferably higher than about 99.99%. Such purity determination may be made by any method known to those skilled in the art, such as chiral HPLC analysis or chiral electrophoresis analysis.

As used herein, the term ED₅₀ refers to the effective dose that produces a given effect in 50% of the subjects.

As used herein, a “disease” is astute of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” of a compound is that amount of compound that is sufficient to provide a beneficial effect to the subject to which the compound is administered. The term to “treat,” as used herein, means reducing the frequency with which symptoms are experienced by a patient or subject or administering an agent or compound to reduce the severity with which symptoms are experienced.

As used herein, “treating a disease or disorder” means reducing the frequency with which a symptom of the disease or disorder is experienced by a patient. Disease and disorder are used interchangeably herein.

As used herein, the term “adverse events” (AEs) or “adverse effects” refer to a change in normal behavior or homeostasis and refers to observed or measured effects in animals such as hunching posture, increased urination and defecation, clonic movements and other seizure-like behaviors, pronounced drops in mean arterial blood pressure, production of cardiac arrhythmias and death.

### Compositions of the Invention

In one aspect, the invention includes a composition comprising (+)-doxapram or a salt thereof wherein the composition is essentially free of (−)-doxapram or a salt thereof.

In one embodiment, the enantiomeric purity of the (+)-doxapram or a salt thereof is at least about 90%. In another embodiment, the enantiomeric purity of the (+)-doxapram or a salt thereof is at least about 95%. In yet another embodiment, the enantiomeric purity of the (+)-doxapram or a salt thereof is at least about 97%. In yet another embodiment, the enantiomeric purity of the (+)-doxapram or a salt thereof is at least about 99%. In yet another embodiment, the enantiomeric purity of the (+)-doxapram or a salt thereof is at least about 99.5%. In yet another embodiment, the enantiomeric purity of the (+)-doxapram or a salt thereof is at least about 99.9%. In yet another embodiment, the enantiomeric purity of the (+)-doxapram or a salt thereof is at least about 99.95%. In yet another embodiment, the enantiomeric purity of the (+)-doxapram or a salt thereof is at least about 99.99%. In yet another embodiment the invention includes salts of (+)doxapram comprising specific and medically useful crystalline polymorphic forms, as characterized by techniques known in the art, including by not limited to X-ray powder diffraction analysis (XRPD) and differential scanning calorimetry (DSC). In yet another embodiment, the composition further comprises at least one pharmaceutical carrier.

### Methods of the Invention

The invention includes a method of preparing a composition comprising (R)-doxapram, wherein in the composition (R)-doxapram is in an enantiomeric excess over (S)-doxapram. The invention comprises the step of reacting a chiral acid with a first solution comprising doxapram, to generate a first system, wherein (S)-doxapram and the chiral acid form a salt that is insoluble in the first system. The invention further comprises the step of isolating a second solution from the first system. The invention further comprises the step of preparing a composition from the second solution, wherein in the composition (R)-doxapram is at an enantiomeric excess over (S)-doxapram.

In one embodiment, the chiral acid is a tartaric acid derivative. In another embodiment, the tartaric acid derivative is L-dibenzoyltartaric acid (L-DBTA). In yet another embodiment, the first solution comprises acetone. In yet another embodiment, the enantiomeric excess is greater than about 50% ee.

The invention also includes a method of preparing a composition comprising (R)-doxapram, wherein in the composition (R)-doxapram is in a second enantiomeric excess over (S)-doxapram. The invention comprises the step of reacting a first chiral acid with a first solution comprising doxapram, to generate a first system, wherein (S)-doxapram and the first chiral acid form a salt that is insoluble in the first system. The invention further comprises the step of isolating a second solution from the first system, wherein in the second solution (R)-doxapram is in a first enantiomeric excess over (S)-doxapram. The invention further comprises the step of reacting the second solution with a basic solution, to generate a second system. The invention further comprises the optional step of concentrating the second system, to generate a third system. The invention further comprises the step of dissolving the second or third system in a first organic solvent, to generate a fourth system. The invention further comprises the step of reacting the fourth system with a second chiral acid, to generate a fifth system, wherein (R)-doxapram and the second chiral acid form a salt that is insoluble in the fifth system. The invention further comprises the step of isolating a solid from the fifth system. The invention further comprises the optional step of (i) dissolving the solid isolated from the fifth system in a second organic solvent, to generate a sixth system; (ii) reacting the sixth system with an aqueous basic solution, to generate a seventh system, wherein the pH of the seventh system is equal to or greater than 8; and, (ii) isolating the organic phase from the seventh system. The invention further comprises the step of preparing a composition from the solid isolated from the fifth system or the organic phase isolated from the seventh system, wherein in the composition (R)-doxapram is in a second enantiomeric excess over (S)-doxapram.

In one embodiment, the first chiral acid is a first tartaric acid derivative. In another embodiment, the first tartaric acid derivative is L-dibenzoyltartaric acid (L-DBTA). In yet another embodiment, the second chiral acid is a second tartaric acid derivative. In yet another embodiment, the second tartaric acid derivative is D-dibenzoyltartaric acid (D-DBTA). In yet another embodiment, the first organic solvent comprises acetone. In yet another embodiment, the first enantiomeric excess is greater than about 50% ee. In yet another embodiment, the second enantiomeric excess is greater than about 60% ee. In yet another embodiment, the composition is further recrystallized at least once from a third organic solvent, wherein in the resulting recrystallized material (R)-doxapram is in at least 98% enantiomeric excess over (S)-doxapram. In yet another embodiment, the third organic solvent comprises a mixture of acetone and ethanol. In yet another embodiment, the composition comprises (R)-doxapram D-dibenzoyltartrate.

The invention also includes a method of preparing a composition comprising (R)-doxapram monohydrochloride monohydrate, comprising hydrating solid (R)-doxapram monohydrochloride to generate solid (R)-doxapram monohydrochloride monohohydrate, wherein in the composition (R)-doxapram is in an enantiomeric excess over (S)-doxapram.

In one embodiment, the enantiomeric excess is at least 98% ee. In another embodiment, the (R)-doxapram monohydrochloride is prepared by reacting a solution of (R)-doxapram in a first solvent with a solution of hydrogen chloride in a second solvent, to generate a first system. In yet another embodiment, the first solvent comprises methyl-tert-butyl-ether. In yet another embodiment, the second solvent comprises ethyl acetate. In yet another embodiment, the (R)-doxapram monohydrochloride is insoluble in the first system. In yet another embodiment, the method further comprises the steps of: optionally concentrating the first system, to generate a second system; dissolving the first or second system in an aqueous solvent, to generate a third system; filtering the third system, to generate a first filtrate; and concentrating the first filtrate, to generate solid (R)-doxapram monohydrochloride.

The invention also includes a method of preparing a composition comprising (R)-doxapram, wherein in the composition (R)-doxapram is in an enantiomeric excess over (S)-doxapram, comprising performing chiral chromatography purification of doxapram using a CHIRALPAK® AY column.

In one embodiment, the mobile phase comprises CO₂. In another embodiment, the mobile phase comprises a mixture of CO₂ and an alcohol, such as methanol or ethanol, with or without an additive, such as dimethylethylamine.

(+)-Doxapram or a salt thereof that is essentially free of (−)-doxapram or a salt thereof may be prepared by resolution of racemic doxapram, using a method such as chiral resolution or chiral chromatography (in anon-limiting example, chiral HPLC).

### Enantiomeric Separation By Chiral Chromatography

(+)-Doxapram and (−)-doxapram may be obtained by dissolving the racemate in a suitable solvent and passing the solution through a HPLC column containing a chiral stationary phase. Non-limiting examples of suitable chiral HPLC columns and conditions contemplated within the invention are: (a) a CHIRALPAK® AY g/min, a column, with 3 cm internal diameter×25 cm length, using ethanol with 0.2% DMEA (dimethylethylamine) and CO₂ as mobile phase, in a ratio of 15:85, with a flow rate of 85 g/min, a column temperature of 35° C., and UV detection at 220 nm; and (b) a CHIRALPAK® AY-H column, with 3 cm internal diameter×25 cm length, using ethanol with 0.2% DMEA and CO₂ as mobile phase, in a ratio of 15:85, with a flow rate of 85 g/min, a column temperature of 35° C., and UV detection at 220 nm. The invention is not limited to these examples as other chiral stationary phases, solvents and HPLC conditions are applicable and may be readily developed and utilized by one skilled in the art.

### Enantiomeric Separation By Chiral Resolution

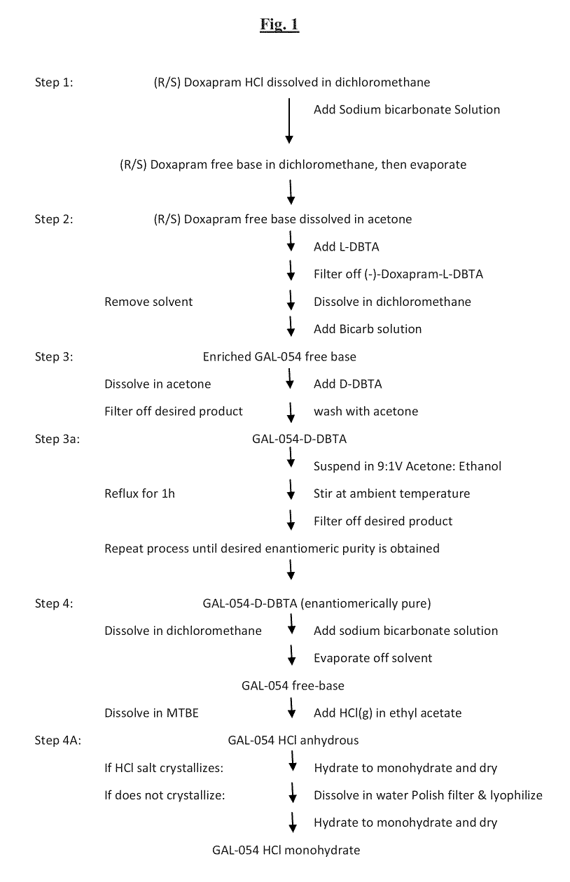

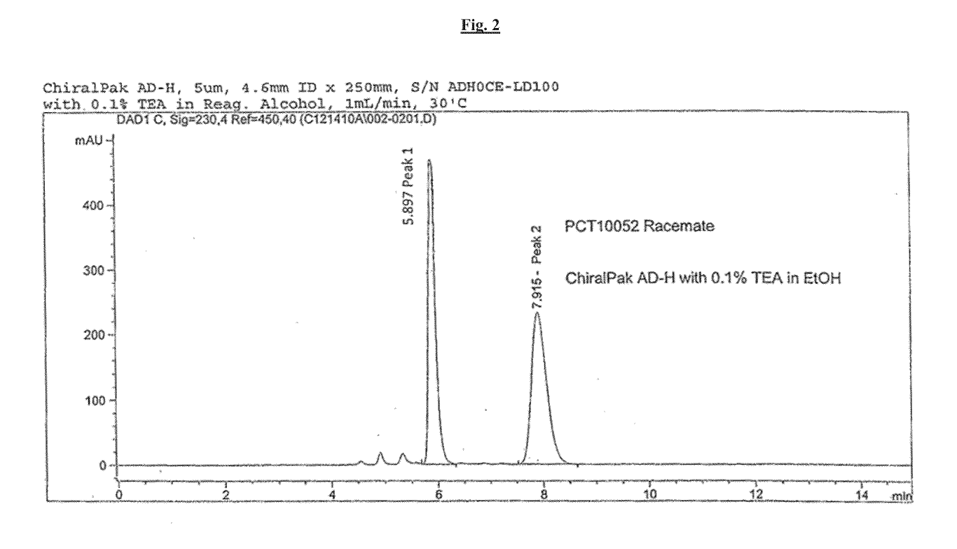

(+)-Doxapram and (−)-doxapram enantiomers may be obtained by a method comprising the steps of dissolving the racemate in a suitable solvent and treating the corresponding solution with a chiral organic acid, such as tartaric acid or a derivative thereof, such as dibenzoyltartaric acid (DBTA). The method further comprises the step of using crystallization to separate the diastereomeric salts. The method further comprises the step of isolating the desired enantiomer via neutralization and extraction (FIG. 1).

### Asymmetric Synthesis:

Doxapram, (+)-doxapram and (−)-doxapram enantiomers may be obtained by a method comprising converting (Z)-butene-1,4-diol into the intermediate (Z)-4-(diphenylacetyl-ethyl-amino)-but-2-enyl ester of acetic acid, and subsequently inducing carbocyclization of the intermediate by reacting it with a strong base, such as potassium hexamethyldisilazide or sparteine, to yield 1-ethyl-3,3-diphenyl-4-vinyl-pyrrolidin-2-one, racemic or optically enriched form respectively. The carbocyclization reaction may be also carried out in the presence of tris(dibenzylideneacetone)dipalladium(0) and BINAP (2,2′-bis(diphenylphosphino)-1,1′-binaphthyl) to obtain 1-ethyl-3,3-diphenyl-4-vinyl-pyrrolidin-2-one in optically enriched form. The intermediate 1-ethyl-3,3-diphenyl-4-vinyl-pyrrolidin-2-one may be further derivatized to yield doxapram, (+)-doxapram or (−)-doxapram enantiomers.

### Chiral Synthesis:

(+)-Doxapram may be obtained by a method comprising alkylating (S)-3-hydroxypyrrolidine to yield (S)-1-ethylpyrrolidin-3-ol, which may be chlorinated to yield (R)-3-chloro-1-ethylpyrrolidine. In one aspect, the chirality of the material is fixed by way of the alkylation reaction and is not affected by subsequent reactions. Therefore, if suitable chiral purity is established at the stage of the alkylation product, it remains intact throughout the remainder of the synthesis. In one embodiment, reaction monitoring for chirality during the synthesis need only be performed through the alkylation stage.

Reaction of (R)-3-chloro-1-ethylpyrrolidine with the anion of diphenylacetonitrile yields (R)-2-(1-ethylpyrrolidin-3-yl)-2,2-diphenylacetonitrile. Subsequent hydrolyses converts the nitrile group to a carboxamide and then to the carboxylic acid (R)-2-(1-ethylpyrrolidin-3-yl)-2,2-diphenylacetic acid. The carboxylic acid may be rearranged to (R)-1-ethyl-4-(2-iodoethyl)-3,3-diphenylpyrrolidin-2-one, without affecting the chirality of the material (Scheme 21).

In another embodiment, (R)-ethyl-3-pyrrolidinol may be converted to a tosylate, mesylate, or other nucleophilically labile hydroxyl derivative known in the art, and alkylated with the anion of diphenylacetonitrile to afford, by inversion of configuration, (R)-2-(1-ethylpyrrolidin-3-yl)-2,2-diphenylacetonitrile (Scheme 22). This compound may be further derivatized to yield (+)-doxapram.

In a further embodiment, the chiral purity of any basic intermediate, such as the alkylation product, may be improved as necessary by forming a salt with a chiral acid and separating the corresponding diastereosiomeric salts. This procedure comprises reacting a chiral acid with a solution comprising the free base of the basic intermediate (such as the alkylation product) to generate a first system, wherein either the (R) enantiomer of the alkylation product, or its (5) enantiomer, selectively crystallizes or remains in solution, allowing partial or complete removal of the undesired enantiomer.

The (−)-doxapram enantiomer may be obtained using the same procedures outlined herein, utilizing (R)-3-hydroxypyrrolidine, (S)-1-ethylpyrrolidin-3-ol or (S)-3-chloro-1-ethylpyrrolidine as starting materials.

### Salts

The compounds described herein may form salts with acids, and such salts are included in the present invention. In one embodiment, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, D-glucuronic, lactic, malic, tartaric, dibenzoyltartaric, dibenzyltartaric, benzoyltartaric, benzyltartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, cyclohexylaminosulfonic, stearic, alginic, β-hydroxybutyric, salicylic, galactaric and galacturonic acid.

In one embodiment, the salts of (+)-doxapram are crystalline polymorphs, as characterized by XRPD, DSC and other technique known in the art. The salts and their crystalline polymorphs may possess medicinally useful solubility profiles, which offer desirable absorption behaviors upon oral administration to humans. Isolation of the salt may also offer a means to remove impurities and result in products that can be conveniently handled in manufacturing environments. The salts contemplated within the invention, as isolated solid materials, may also possess properties which allow for economical manufacturing and which confer unique and valuable stability during isolation, handling, shipping, and storage upon long term storage.

### Pharmaceutical Compositions and Formulations

The invention also includes a pharmaceutical composition of (+)-doxapram or a salt thereof, wherein the composition is essentially free of (−)-doxapram or a salt thereof. Such a pharmaceutical composition may consist of (+)-doxapram or a salt thereof alone, wherein the compositions is essentially free of (−)-doxapram or a salt thereof, in a form suitable for administration to a subject, or the pharmaceutical composition may comprise (+)-doxapram or a salt thereof, wherein the compositions is essentially free of (−)-doxapram or a salt thereof and one or more pharmaceutically acceptable carriers, one or more additional ingredients, or some combination of these. The compound (+)-doxapram may be present in the pharmaceutical composition in the form of a physiologically acceptable salt, such as in combination with a physiologically acceptable anion, as is well known in the art.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier. Pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition art agent which delays absorption, for example, aluminum monostearate gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative, such as benzyl alcohol, sorbic acid, parabens, imidurea or combinations thereof. The composition may also include an antioxidant and a chelating agent to inhibit the degradation of the compound. Preferred antioxidants for some compositions are BHT, BHA, alpha-tocopherol and ascorbic acid. Preferred chelating agents include edetate salts (e.g. disodium edetate) and citric acid.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2, 7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein,

### General Comments

The term “IPC” refers to In-Process Controls: assays that are conducted during the course of the process to monitor progress (e.g., pH, and absence of starting material) and/or to evaluate quality of material (e.g., purity, and enantiomeric excess).

### Example 1

### Chiral Resolution for Preparing (+)-Doxapram

Step 1: Convert (R/S) Doxapram HCl to (R/S) Doxapram free base
Step 2: Preparation of enriched GAL-054 (the desired enantiomer) from (R/S) Doxapram free base

### Step 3: Preparation of GAL-054 D-DBTA from enriched GAL-054

Step 3a: Purification of GAL-054-D-DBTA Salt (the diasteromeric salt)
Step 4: Preparation of GAL-054HCl from GAL-054-D-DBTA Salt
Step 4A: Preparation of GAL-054HCl monohydrate from GAL-054HCl

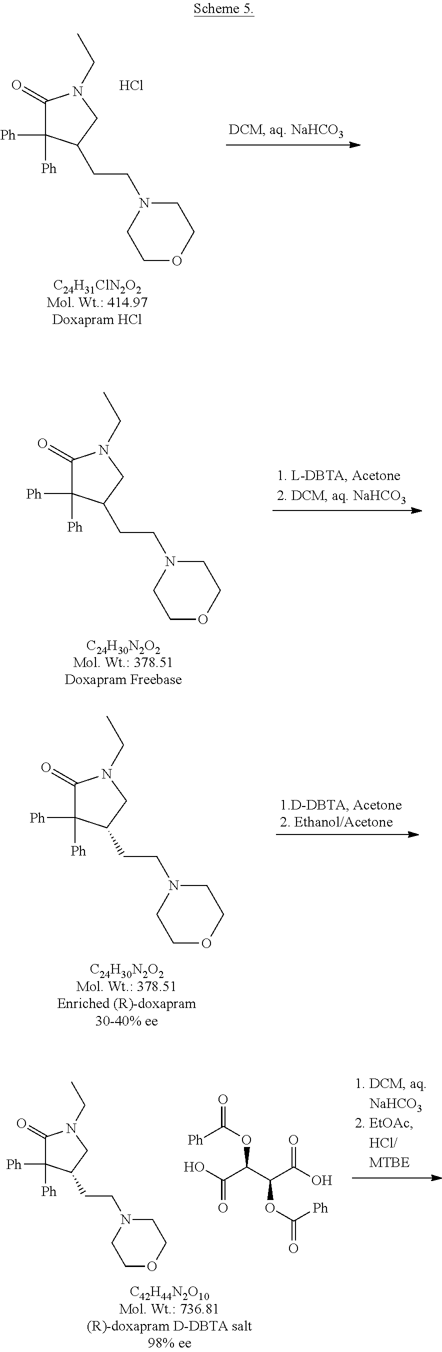 [SMILES: CCN1CC(CCN2CCOCC2)C(c2ccccc2)(c2ccccc2)C1=O ; Cl ; CCN1CC(CCN2CCOCC2)C(c2ccccc2)(c2ccccc2)C1=O ; CCN1C[C@H](CCN2CCOCC2)C(c2ccccc2)(c2ccccc2)C1=O ; CCN1C[C@H](CCN2CCOCC2)C(c2ccccc2)(c2ccccc2)C1=O ; O=C(O[C@H](C(=O)O)[C@H](OC(=O)c1ccccc1)C(=O)O)c1ccccc1]

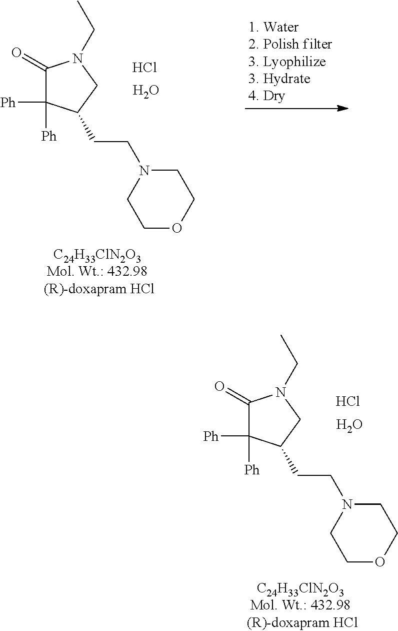

### Step 1: Conversion of Doxapram HCl to Doxapram Free Base

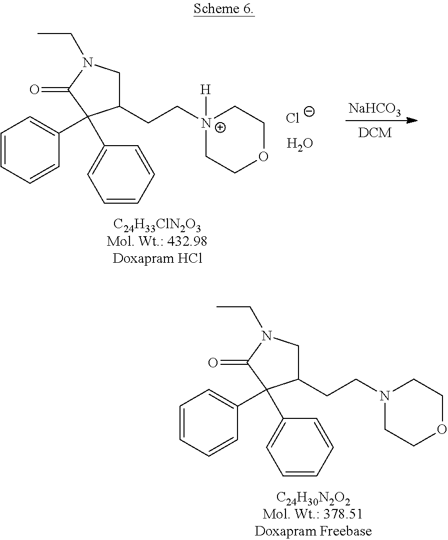

TABLE 1
List of Materials for Step 1.

| | | | | | Expected | Purpose of |
|---|---|---|---|---|---|---|
| Reagents/Materials | MW | Eqs. | Moles | Density | Amt (kg) | Material |
| Doxapram HCl USP | 432.98 | 1.0 | 2.3 | — | 1.0 | Racemic |
| | | | | | | Drug |
| Dichloromethane, ≧99% | 84.93 | — | — | 1.33 | 16 | Solvent |
| 4.5% Sodium Bicarbonate, | 84.01 | 1.3 | 3.0 | 1 | 5.6 | Acid |
| aqueous solution | | | | | | neutralizer |
| Sodium Sulfate, anhydrous | 142.04 | — | — | — | To be | Remove |
| | | | | | determined | water from |
| | | | | | by chemist | solvent |

Doxapram hydrochloride was placed in dichloromethane and the mixture was stirred with the sodium bicarbonate solution [IPC 1 & 2]. After separation from the aqueous layer, water was removed from the dichloromethane layer by adding anhydrous sodium sulfate and then filtering off the solid. Doxapram free base was isolated by roto-evaporation of the organic filtrate [IPC 3 & 4].

### Step 1 In-Process Controls (IPC):

IPC Test 1: Measure pH of 4.5% sodium bicarbonate solution following extractions; Maintain pH ≧8.0 to maintain extraction efficiency
IPC Test 2: IPC achiral HPLC analysis of 4.5% sodium bicarbonate solution following extractions; Report results for process development information; Look for detectable levels of doxapram.
IPC Test 3: IPC achiral HPLC analysis of rotary evaporator content; Report results
IPC Test 4: Gross weight of doxapram free base; Report results
   Step 2: Preparation of Enriched (+)-Doxapram from (R/S)-Doxapram Free Base

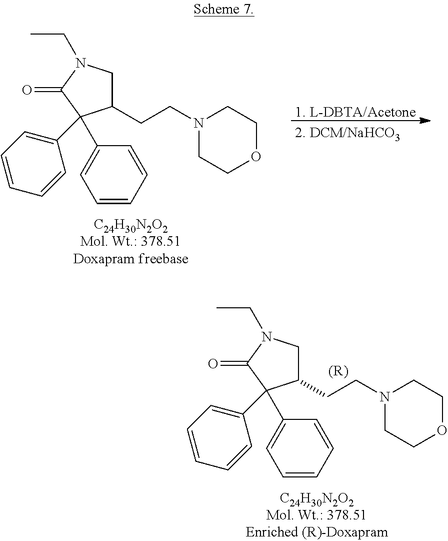

TABLE 2
List of Materials for Step 2.

| | | | | | Expected | Purpose of |
|---|---|---|---|---|---|---|
| Reagents/Materials | MW | Eqs. | Moles | Density | Amt (kg) | Material |
| Doxapram freebase | 378.51 | 1.0 | 2.3 | — | 0.87* | Racemate drug |
| Dibenzoyl-L-tartaric | 358.30 | 0.438 | 1.01 | — | 0.36 | Complex with |
| acid (L-DBTA), ≧98% | | | | | | undesired |
| | | | | | | enantiomer & |
| | | | | | | precipitate from |
| | | | | | | solution |
| Dichloromethane, ≧99% | 84.93 | — | — | 1.33 | 7.5 | Solvent |
| 9% Sodium Bicarbonate, | 84.01 | 0.68 | — | 1 | 2.0 | Break |
| aqueous solution | | | | | | diastereomeric |
| | | | | | | salt to form free |
| | | | | | | base |
| Sodium Sulfate, | 142.04 | — | — | — | To be | Remove residual |
| anhydrous | | | | | determined | water in solvent |
| | | | | | by chemist | |
| Acetone, ≧99% | 58.08 | — | — | 0.79 | 10 | Solvent |

*if the Step 1 isolated yield was >100%, amount here represented theoretical amount of that step

The two enantiomers were each present as approximately 50% of doxapram free base. L-DBTA was added in two portions to form a salt with (S)-doxapram and precipitate it from solution. Feasibility experiments have determined that the quantity of L-DBTA necessary to remove the (S)-enantiomer without significantly precipitating the (R)-enantiomer is approximately 88% of an equivalent quantity of (S)-doxapram present.

To a multi-neck RB flask, 0.87 kg of doxapram free base and 6.1 L of acetone were added and the mixture was stirred. Then, L-DBTA (0.21 kg) was added at 20° C. and the resultant mixture was stirred for 2 hours. In a separate container, L-DBTA (0.15 kg) were dissolved in 0.2 L of acetone and this solution was slowly added to the growing suspension in the multi-neck RB flask over 3 hours. The addition funnel was rinsed with acetone, and the mixture was stirred for one additional hour. The precipitate was filtered off, and the cake was rinsed with 0.5 L of acetone. Samples of solid and liquid were collected for analysis [IPC 1 & 2]. The filter cake ((S)-doxapram) was vacuum-dried and stored [IPC 6]. The filtrate was concentrated by rotary evaporation.

The material, enriched in (R)-doxapram, was dissolved in dichloromethane (2.6 L plus 1 L flask rinse) and transferred to a separatory flask. To this solution, 2 L of 9% sodium bicarbonate solution were slowly and carefully added. The mixture was agitated for at least 1 hour, and the pH was checked. If the pH dropped below pH 8, additional bicarbonate solution was added as needed. The organic dichloromethane layer was collected. The separation flack was then charged with an additional 1 L of dichloromethane and agitated for at least 10 minutes to extract additional product from the aqueous bicarbonate layer. The organic layer was collected. The bicarbonate solution was collected [IPC 3] and a sample analyzed for (R)-doxapram content. To the combined organic DCM layers was added 1 kg of anhydrous magnesium sulfate, and the mixture was then filtered and the volatiles removed by rotary evaporation to consolidate the enriched (R)-doxapram free base. A sample of this material was obtained for analysis [IPC 4 & 5].

### Step 2 In-Process Controls:

IPC Test 1: Chiral HPLC of enriched (R)-doxapram in acetone; Report results
IPC Test 2: Chiral HPLC of (S)-doxapram-L-DBTA salt; Report results
IPC Test 3: Achiral HPLC of aqueous bicarbonate layer; Report results
IPC Test 4: Chiral HPLC of enriched (R)-doxapram (following rotary evaporation); Report results
IPC Test 5: Loss on drying (105° C. for 2 hours) of enriched (R)-doxapram (following rotary evaporation); Report results
IPC Test 6: Chiral HPLC of (S)-doxapram-L-DBTA salt (following vacuum oven drying); Report results
   Step 3a: Preparation of (R)-Doxapram D-DBTA from Enriched (R)-Doxapram

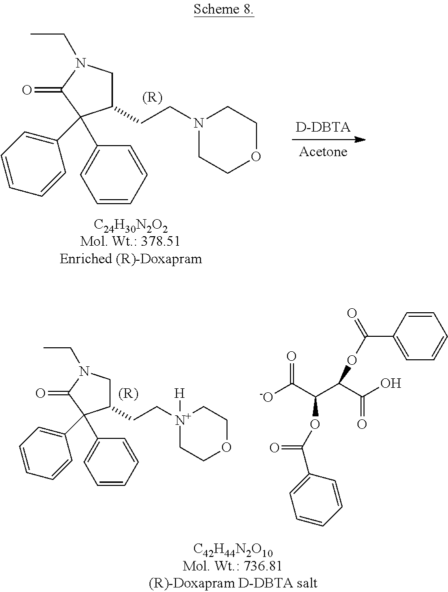

TABLE 3
List of Materials for Step 3a.

| | | | | | Expected | Purpose of |
|---|---|---|---|---|---|---|
| Reagents/Materials | MW | Eqs. | Moles | Density | Amt (kg) | Material |
| Enriched (R)-doxapram | 378.8 | 1 | 1.6 | — | 0.60 | Starting material |
| | | | | | | containing 2:1-4:1 |
| | | | | | | R to S |
| | | | | | | enantiomer ratio of |
| | | | | | | Doxapram |
| Dibenzoyl- | 358.31 | 0.72* | 1.1* | — | 0.41 (to be | Chiral salt to |
| D-tartaric acid, 98% | | | | | determined | preferentially |
| | | | | | by chemist) | complex with R |
| | | | | | | enantiomer |
| Process Water, Filtered | 18.02 | — | — | 1 | | Solvent |
| Acetone, ≧99% | 58.08 | — | — | 0.79 | 3.8 | Solvent |

In this step, D-DBTA was added to the system, forming a salt with (R)-doxapram, and precipitating it from solution. Into a multineck RB flask was added the enriched (R)-doxapram (0.6 kg) along with 4.2 L acetone. The mixture was vigorously stirred and to this solution were added 0.25 equivalents of D-DBTA. The quantity of additional D-DBTA to be added was calculated based on the (R)-doxapram free base content determined in Step 2 IPC 4&5; total D-DBTA added was 0.65-0.8 eq (in one embodiment, depending on operator discretion). The D-DBTA was dissolved in acetone and added to the RB flask slowly over 3 hours. The resultant solid was filtered, and the cake was washed with acetone. Samples of the filtrate and cake were obtained for testing [IPC 1 & 2]. The filtrate was agitated for at least 30 minutes in the round bottom flask. The precipitate produced was filtered and the cake was rinsed with acetone. Samples of the cake and the rinse were obtained for testing [IPC 3 & 4]. The round bottom flask was rinsed with 0.5 L acetone and filtered. The agitation and filtration process was repeated as needed [IPC 5, 6 & 7].

### Step 3a In-Process Contras—

IPC Test 1: Chiral HPLC of (R)-doxapram-D-DBTA Salt (filter cake 1st); Report results
IPC Test 2: Chiral HPLC of (R)-doxapram-D-DBTA Salt (filtrate 1st); Report results
IPC Test 3: Chiral HPLC of (R)-doxapram-D-DBTA Salt (filter cake 2nd); Report results
IPC Test 0.4: Chiral HPLC of (R)-doxapram-D-DBTA Salt (filtrate 2nd); Report results
IPC Test 5: Chiral HPLC of Acetone filtrates; Report results
IPC Test 6: Chiral HPLC of (R)-doxapram-D-DBTA salt; Report results
IPC Test 7: Loss on Drying (105° C. for 2 hours) of (R)-doxapram-D-DBTA salt; Report results

### Step 3b: Purification of (R)-Doxapram-D-DBTA Salt

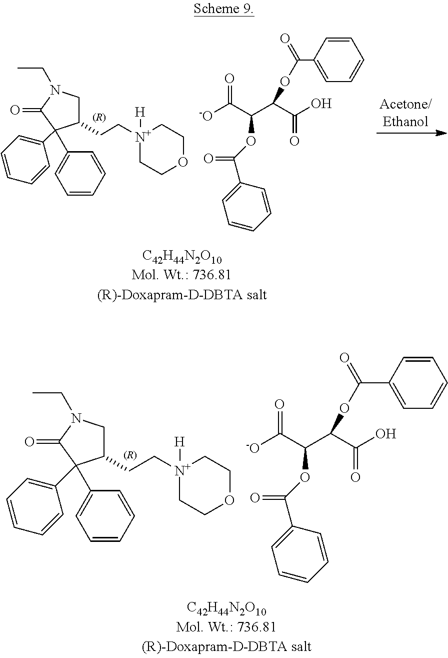

TABLE 4
List of Materials for Step 3b.

| | | | | | Expected | Purpose of |
|---|---|---|---|---|---|---|
| Reagents/Materials | MW | Eqs. | Moles | Density | Amt (kg) | Material |
| (R)-doxapram- | 736.81 | — | — | — | all wet cake | 60-90% ee pure |
| D-DBTA salt | | | | | available | (R)-doxapram- |
| | | | | | from | D-DBTA salt |
| | | | | | previous step | |
| (9v/1v) Acetone (≧99%)/ | — | — | — | — | — | Selective |
| Ethanol (≧99%) | | | | | | recrystallization |
| | | | | | | solvent system |

(R)-doxapram D-DBTA was enantiomerically purified by dissolution and precipitation from acetone/ethanol. Into a multineck round bottom flask, acetone ≧99% ethanol ≧99% (9/1 (v/v)) were added to the wet product of Step 3a and the mixture was agitated. The resultant mixture was heated at reflux (approximately 60° C.) for at least 1 hour. Then, the mixture was cooled to approximately 20° C. and agitated for at least an additional hour. The mixture was filtered and the round bottom flask was rinsed onto the filter cake with 0.5 L of acetone/ethanol (9/1 (v/v). Samples of the filter cake and of the filtrate were obtained for chiral assay [IPC 1 & 2].

If the filter cake was <98.5% ee, then step 3b was repeated until an enantiomeric purity of ≧98.5% ee [IPC 1 & 2] was achieved. If the filter cake was found to be ≧98.50 cc (enantiomeric purity), then the material was vacuum oven dried (vacuum >20 inches of Hg) on oven paper at 40° C., until constant weight for one hour [IPC 3] was obtained. A dried sample is then obtained [IPC 4].

### Step 3b In-Process Controls:

IPC Test 1: Chiral HPLC of (R)-doxapram-D-DBTA Salt in Acetone/Ethanol filtrates; Report results
IPC Test 2: Chiral HPLC of (R)-doxapram-D-DBTA Salt (wet cake); Limits: NL 98.5% ee [Calculated by % (R)-doxapram minus % (S)-doxapram]
IPC Test 3: Weight monitoring during vacuum oven drying of (R)-doxapram-D-DBTA Salt; Limits: Dry to constant weight. Constant weight defined as tray reading at least 1 hour apart having the same weight within ±50 g
IPC Test 4: Chiral HPLC of (R)-doxapram-D-DBTA Salt (vacuum oven dried); Limits: NL 985% ee
   Step 4: Preparation of (R)-Doxapram Hydrochloride Monohydrate from (R)-Doxapram-D-DBTA Salt

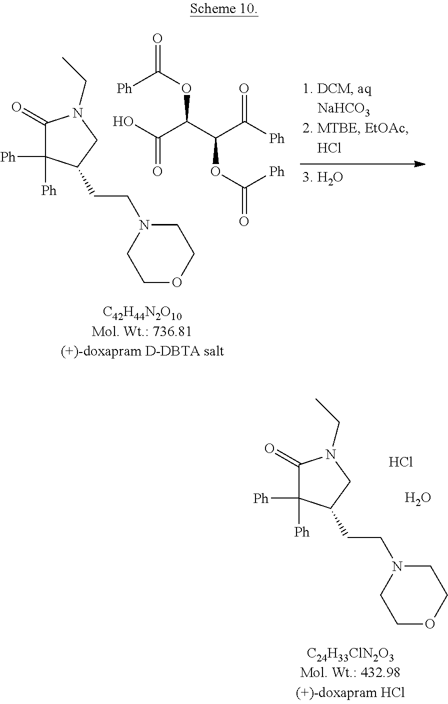

TABLE 5
List of Materials for Step 4.

| | | | | | Expected | Expected |
|---|---|---|---|---|---|---|
| Reagents/Materials | MW | Eqs. | Moles | Density | Amt (kg) | Amt (L) |
| (R)-doxapram D-DBTA salt | 736.81 | 1 | 0.68 | — | 0.50 | — |
| Dichloromethane, ≧99% | 84.93 | — | — | 1.33 | 7.8 | 5.9 |
| 4.5% Sodium Bicarbonate, | 84.01 | — | — | 1 | 5.1* | 5.1* |
| aqueous solution | | | | | | |
| Methyl tert-Butyl Ether | 88.15 | — | — | 0.74 | 10.7 | 14.5 |
| (MTBE), ≧99% | | | | | | |
| Hydrogen chloride gas | 36.46 | — | — | — | * | * |
| Ethyl Acetate, ≧99% | 88.1 | — | — | 0.90 | * | * |
| Water For Injection | 18.02 | — | — | 1 | 1.4 | 1.4 |
| Quality Water | | | | | | |

*TBD—To Be Determined By Chemist

(R)-doxapram D-DBTA was converted to (R)-doxapram hydrochloride monohydrate salt. In a separator flask, 3.5 L of dichloromethane (DCM) and 0.5 kg (R)-doxapram-D-DBTA were added and the mixture was agitated. To this mixture were slowly added 3.4 L of 4.5% sodium bicarbonate, followed by careful agitation for 1 hour. The was maintained at ≧8, and the organic DCM layer was collected. The aqueous phase was extracted again by charging separatory flask with 1.4 L of DCM and agitating for at least 10 minutes. The organic DCM layer was collected. Separately the aqueous phase layer was collected. The empty separatory flask was charged with the previously collected R)-doxapram in DCM layer. To this were slowly added 1.7 L of 4.5% sodium bicarbonate solution, and the mixture was carefully agitated at least 10 minutes. The organic DCM layer, containing the (R)-doxapram, was collected. The aqueous layer was separately collected. The empty separator) flask was charged with the DCM containing 115 the (R)-doxapram. To this were slowly added 1.4 L of water for injection USP (or EP) and the mixture was agitated for at least 10 minutes. After this time, the two layers were separately collected. The DCM layer containing the (R)-doxapram was concentrated via rotary evaporation rinsing flasks with DCM. After evaporation, methyl-t-butyl ether was added and the mixture was subjected to rotary evaporation multiple times to remove residual water, yielding (R)-doxapram as a free base. Samples to determine residual water content [IPC 1] were obtained.

Separately, a multi-neck RB flask was charged with 1 L of ethyl acetate and cooled to 10° C. At this time, HCl gas was bubbled into the flask in order to charge with approximately 40 g of HCl gas. The temperature was maintained at ≦15° C. A 25 mL sample of the approximately 1N HCl in ethyl acetate was obtained for titration [IPC 2].

Into a flask containing the (R)-doxapram obtained as described above were added 5 L of MTBE and the mixture was agitated to effect dissolution. This solution was then charged to a clean multi-neck RB flask, rinsing with 1 L of MTBE. The solution was agitated, and then 1.1 eq of 1N HCl in ethyl acetate were added. The mixture was further agitated at 20° C.±5° C. for 1 hour. Crystallization of (R)-doxapram HCl was verified and the drug substance was filtered, rinsing the flask and wash cake with an additional 1 L of MTBE.

The resultant powder was vacuum oven-dried on trays pre-lined with fluorocarbon film, to constant weight at ≦55° C. Samples were obtained to monitor residual solvent level [IPC 3]. The vacuum oven may be heated to <110° C. if necessary to remove excess MTBE [IPC 3]. The residual water content was measured; in one embodiment, the monohydrate was the form obtained [IPC 4]. If needed, material may dried to within water range of a monohydrate. If needed, an empty clean vacuum tray was charged with filtered process water, positioned on bottom shelf of vacuum oven and an internal temperature ≦30° C. was maintained for at least 8 hours to produce the monohydrate final product [IPC 5]. The bulk API was stored in glass jars under quarantine while proceeding with finish product release tests.

### Step 4 In-Process Controls—

IPC Test 1: Water determination by Karl Fisher of (R)-doxapram from the rotary evaporator flask (containing residual MTBE); Report results—expected range ≦0.1%
IPC Test 2: Titration of approximate 1N HCl in ethyl acetate; limits: 0.7 to 1.3N

IPC Test 3: Preliminary residual solvent analysis of (R)-doxapram HCl (powder in vacuum oven); Limits: MTBE, ethanol, ethyl acetate and acetone limit NMT 1000 ppm each; DCM limit NMT 300 ppm.
IPC Test 4: Water determination by Karl Fisher of (R)-doxapram (powder in vacuum oven); Limits: 3.7-4.8%
IPC Test 5: Water determination by Karl Fisher of (R)-doxapram (powder in vacuum oven); Limits: 3.7-4.8%
   Step 4a: Preparation of (R)-Doxapram HCl Monohydrate from (R)-Doxapram HCl

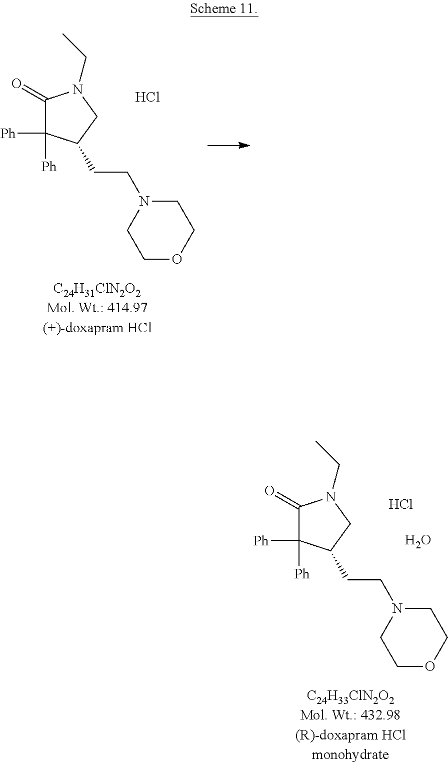 [SMILES: CCN1C[C@H](CCN2CCOCC2)C(c2ccccc2)(c2ccccc2)C1=O ; Cl ; CCN1C[C@H](CCN2CCOCC2)C(c2ccccc2)(c2ccccc2)C1=O ; Cl ; O]

TABLE 6
List of Materials for Step 5.

| | | | | | Expected |
|---|---|---|---|---|---|
| Reagents/Materials | MW | Eqs. | Moles | Density | Amt |
| (R)-doxapram HCl | 432.98 | 1.0 | — | — | 100 g |
| Water For Injection | 18.02 | — | — | 1 | * |
| Quality Water, WFI | | | | | |
| Process Water, Filtered | 18.02 | — | — | 1 | * |

* To be determined by chemist

If (R)-doxapram HCl was found to precipitate from MTBE in the previous step, this step may be used to form the crystalline monohydrate, Crystallization of (R)-doxapram HCl from MTBE may be very sensitive to residual water in the solvents. If (R)-doxapram HCl failed to precipitate in the previous step, then the residual solvents may have to be removed and the (R)-doxapram HCl dissolved in water for filtration and lyophilization. (R)-doxapram was produced using both methods: direct crystallization from MBTE; and concentration of non-crystalline (oil) product, which was subsequently converted to the desired (R)-doxapram monohydrate.

If (R)-doxapram HCl precipitated from MTBE in Step 4, following reducing the solvent level to acceptable levels, filtered water was placed into an empty tray in the vacuum oven. Under a nitrogen headspace, the vacuum oven was heated to 30° C. and maintained thereafter, to humidify the chamber and drug substance for 1 hour. This procedure was repeated until the drug substance powder samples stabilized between 3.7 and 4.8% water content [IPC 1]. The water tray was then removed and the vacuum oven jacket temperature heated to 60° C. and maintained thereafter for 8 hours. After allowing to cool to room temperature, drug substance was collected. Samples of drug substance powder were obtained for water determination [IPC 2].

If (R)-doxapram HCl failed to precipitate from MTBE in Step 4, the mixture was then evaporated to dryness to form an oil. This oil was dissolved in minimal volumes of MTBE and evaporated to dryness, and the process was repeated several times to form a powder. Since the solvents contained small amounts of hydrophobic residues, the drug substance was then dissolved in a minimal amount of sterile water and filtered through hydrophobic membrane filters until the aqueous solution was clear [IPC 3)]. The aqueous solution was lyophilized to a fine powder [IPC 4]. The residual water content was measured [IPC 5]. If needed, material was dried to within water range. If needed, an empty clean vacuum tray was charged with filtered process water and positioned on bottom shelf of vacuum oven. The internal temperature was maintained at ≦30° C. for at least 8 hours (IPC 6). The bulk material was collected and stored in amber glass jars under quarantine white proceeding with finish product release tests.

### Step 4a In-Process Controls—

IPC Test 1 & 2: Water determination by Kart Fisher of (R)-doxapram (powder in vacuum oven); Limits: 3.7±4.8%
IPC Test 3: Visual inspection of filtered aqueous solution—clear solution
IPC Test 4: Dry to contrast tray weight when tray temperature is ≧20° C.—constant weight <50 g change within 1 hour
IPC Test 5 & 6: Water determination by Kart Fisher of (R)-doxapram (powder in vacuum oven); Limits: 3.7±4.8%

### Example 2

### Chiral HPLC Separation of Doxapram into (R)-Doxapram and (S)-Doxapram

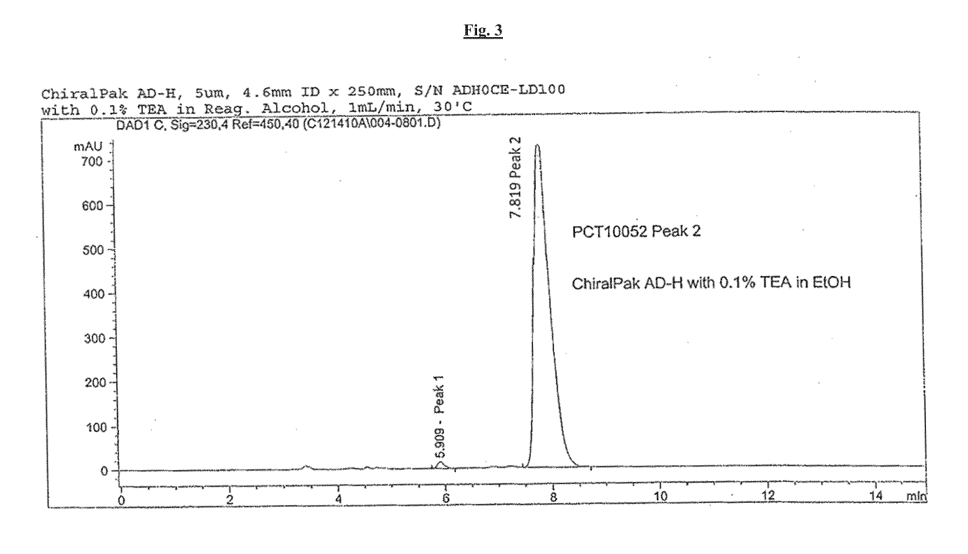

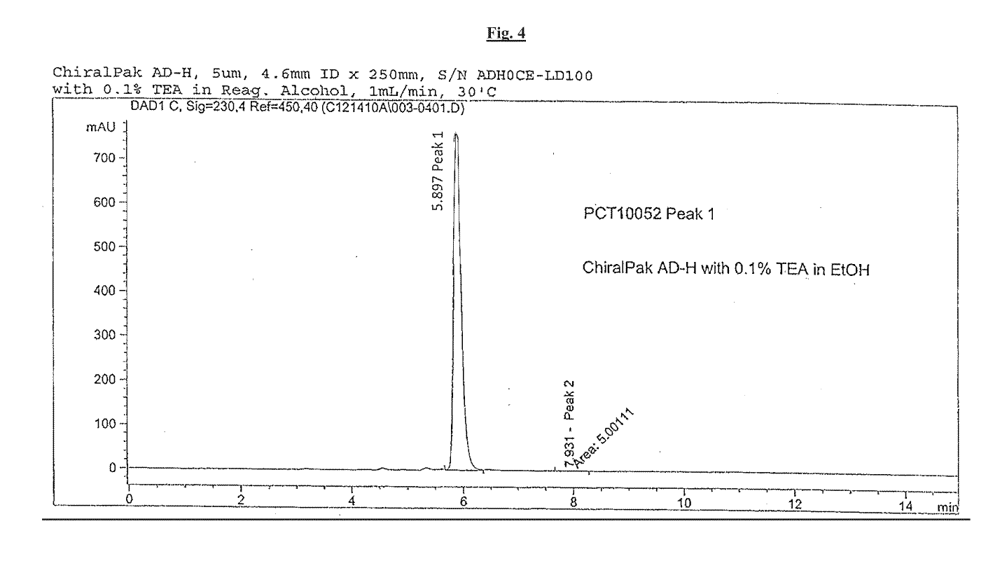

FIGS. 2-4 illustrate chromatograms for doxapram and the two isolated enantiomers of doxapram, which were column separated as the free base.

### Example 3

### Elucidation of Structural Data

### NMR Spectroscopy

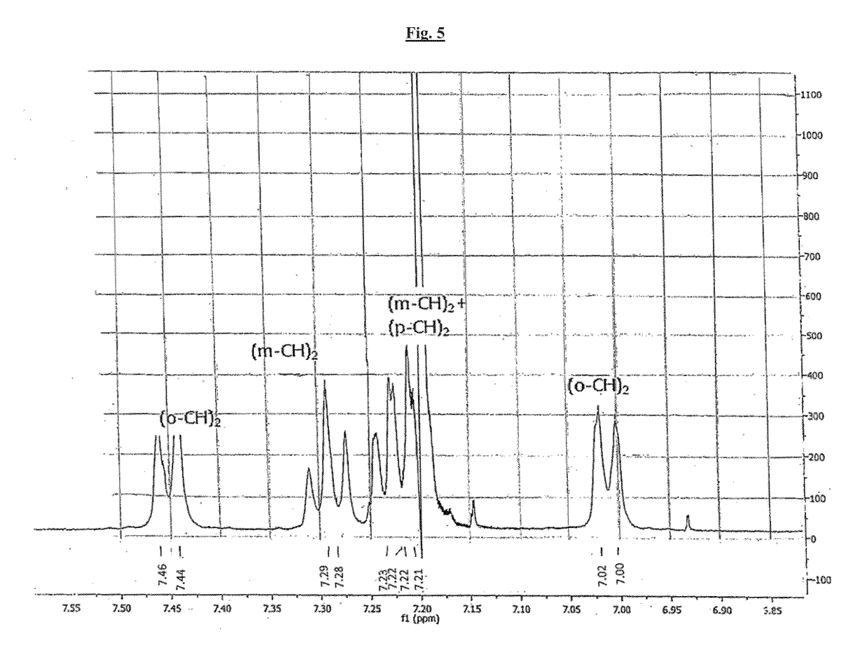

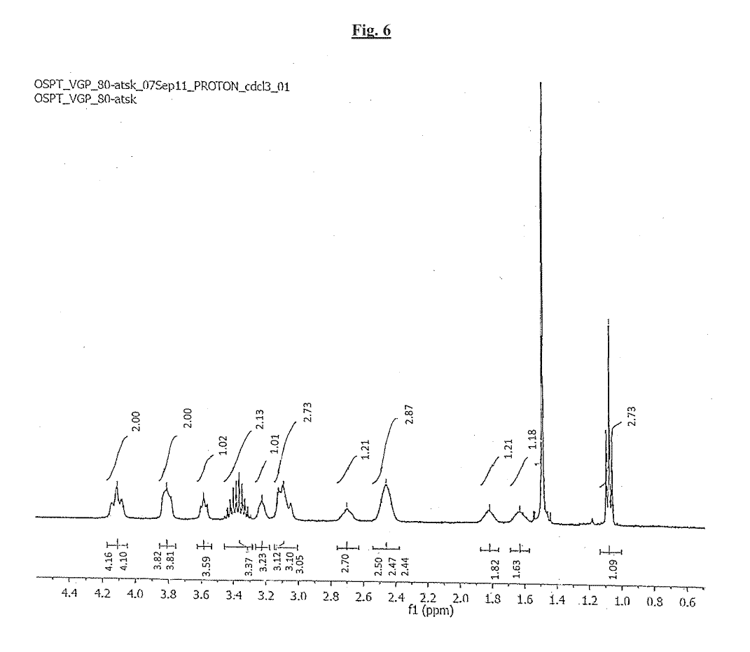

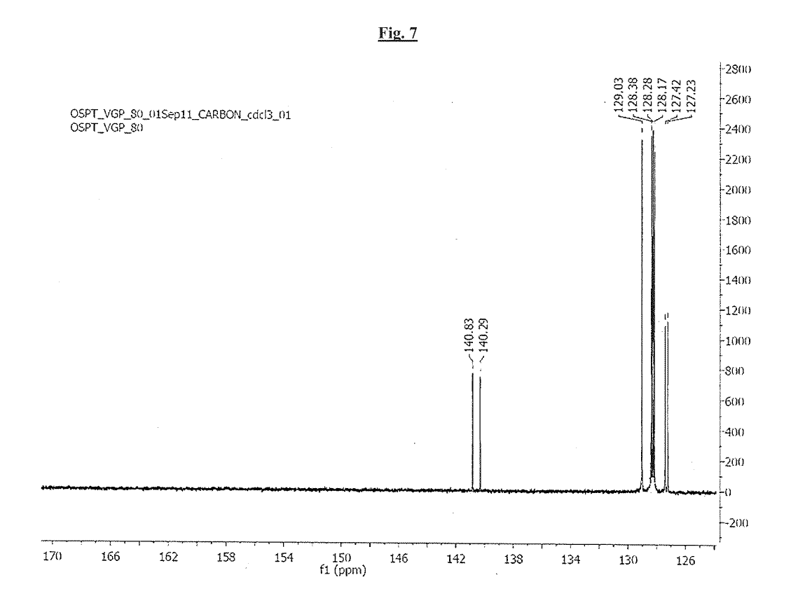

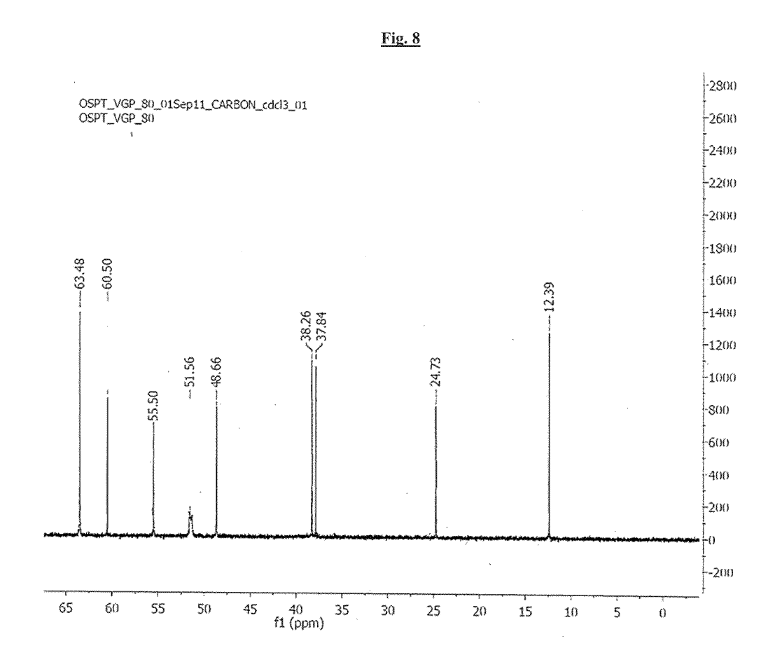

¹H and ¹³C NMR spectra of the (+)-enantiomer of doxapram, along, with putative resonance peak assignments, is illustrated in Tables 7-8 and FIGS. 5-8.

TABLE 7
¹H Chemical Shift Assignments for (+)-doxapram in CDCl₃ at 25° C.

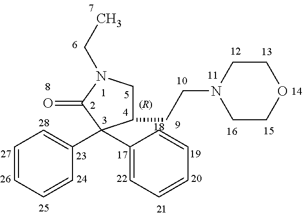

| No. | ¹H (ppm) | Assignment |
|---|---|---|
| A | 1.09 | 7 (3H) |
| B | 1.63, 1.82 | 9 (2H) |
| | 1.7 | Water |
| C | 2.44, 2.47, 2.50, 2.70 | 10, 12, 16 (4H) |
| D | 3.05, 3.10, 3.12, 3.23 | 5, 12, 16, 4 (4H) |
| E | 3.37 | 6 (2H) |
| F | 3.59 | 5 (1H) |
| G | 3.81, 3.82 | 13, 15 (2H) |
| H | 4.10, 4.16 | 13, 15 (2H) |
| I | 7.00, 7.02, 7.21, 7.22, 7.22, | 18-22 and 24-28 (10H) |
| | 7.23, 7.28, 7.29, 7.44, 7.46 | |
| J | 12.96 | 11 (N—H) (1H) |

TABLE 8
¹³C Chemical Shift Assignments for (+)-doxapram in CDCl₃ at 25° C.

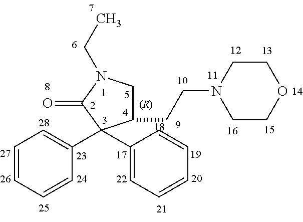

| No | ¹³C (ppm) | Assignment |
|---|---|---|
| A | 12.27 | 7 |
| B | 24.73 | 9 |
| C | 37.84, 38.26 | 4, 6 |
| D | 48.55 | 5 |
| E | 51.29, 51.50 | 12, 16 |
| F | 55.35 | 10 |
| G | 60.50 | 3 |
| H | 63.48 | 13, 15 |
| I | 127.23, 127.42 128.17, 128.28, 128.38, 129.03 | 18-22 and 24-28 |
| J | 140.29, 140.83 | 17, 23 |
| K | 173.49 | 2 |

### IR Spectroscopy

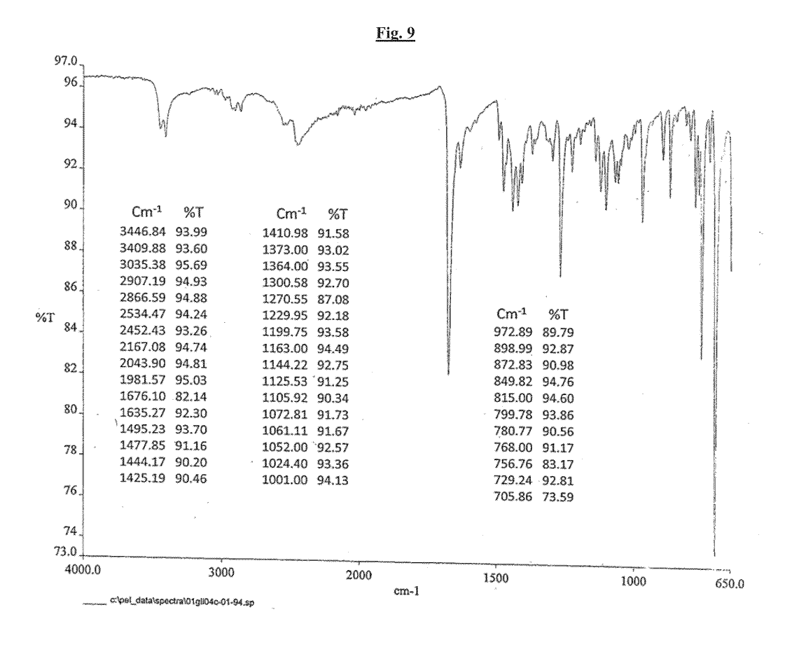

The infrared spectrum of (R)-doxapram is illustrated in FIG. 9. Tentative assignments of absorbance peaks are illustrated in Table 9.

TABLE 9
IR assignments for (R)-doxapram hydrochloride monohydrate

| Wavenumber (cm⁻¹) | Peak Strength | Assignment |
|---|---|---|
| 3448 & 3410 | Weak | C—H stretch |
| 1676 | Strong | C═O amide stretching |
| 1496, 1478, | Weak | C—C ring stretching |
| 1444, 1425 | | |
| 1270 | Medium | C—N stretch |
| 873 | Weak-medium | N—C—H stretch |
| | | (morpholinyl) |
| 756 | Strong-medium | C—H out-of-plane |
| | | aromatic ring bending |
| 706 | Strong | C—C ring bending |

### Mass Spectrometry

The parent ion mass obtained from LCMS was 379 amu, which agrees with the theoretical mass of (R)-doxapram and conforms to the molecular weight of (R)-doxapram. High-resolution results Obtained through direct injection are illustrated in Table 10.

TABLE 10
Mass Spectrometry for (R)-Doxapram (Lot#: VGP-80).

| Determined | Theoretical Exact | |
|---|---|---|
| Molecular Weight | Mass | Formula |
| 379 | 378.51 | C₂₄H₃₀N₂O₂ |

### Elemental Analysis

Elemental analysis results of (R)-doxapram are illustrated in Table 11.

TABLE 11
Elemental Analysis for (R)-Doxapram Hydrochloride
Monohydrate, Lot# VGP-80.

| Element | Theoretical (%) | Result (%) |
|---|---|---|
| C | 66.57% | 66.47% |
| H | 7.68% | 7.59% |
| N | 6.47% | 6.47% |

Thermal Analysis by Differential Scanning calorimetry (DSC)

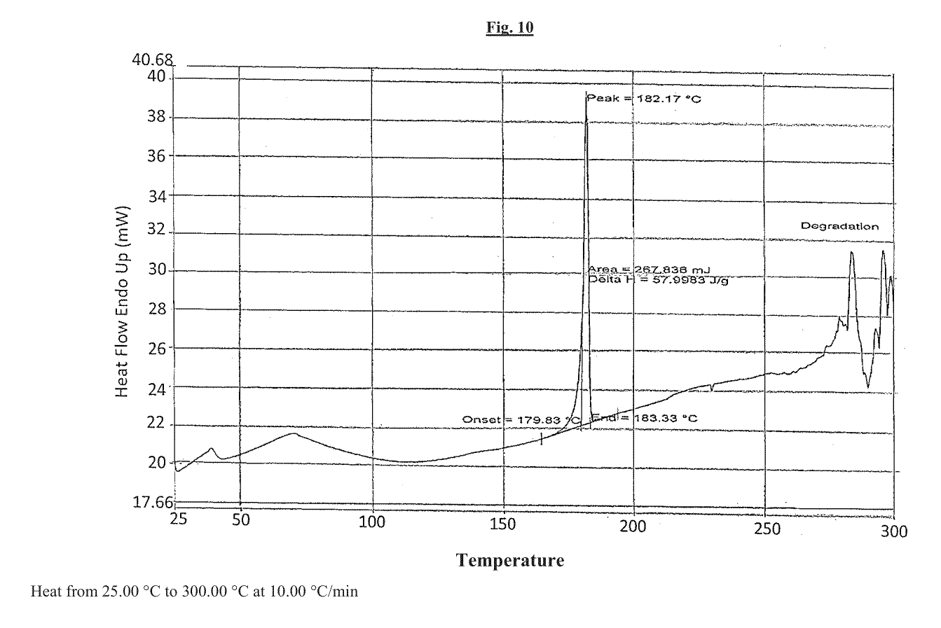

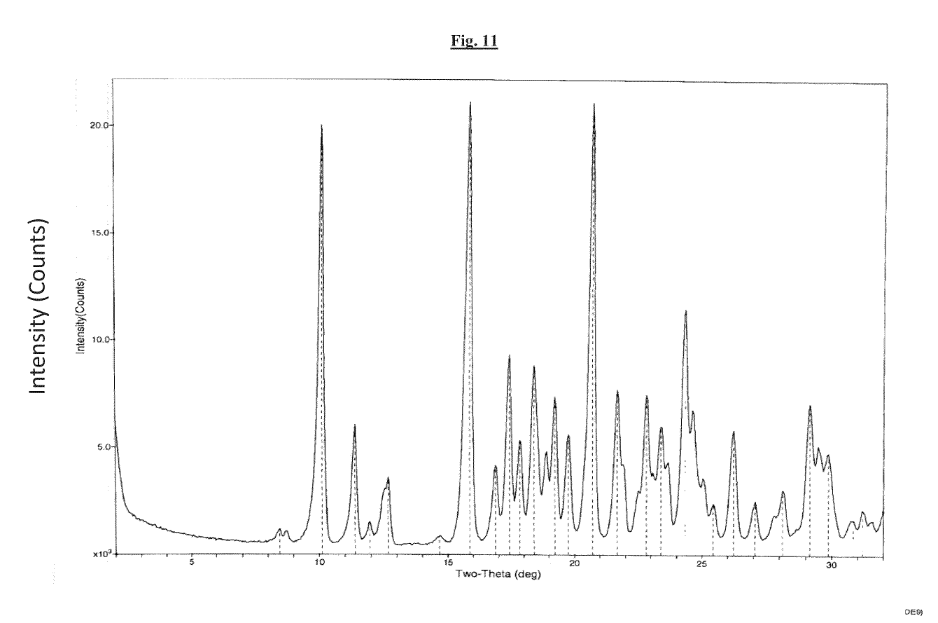

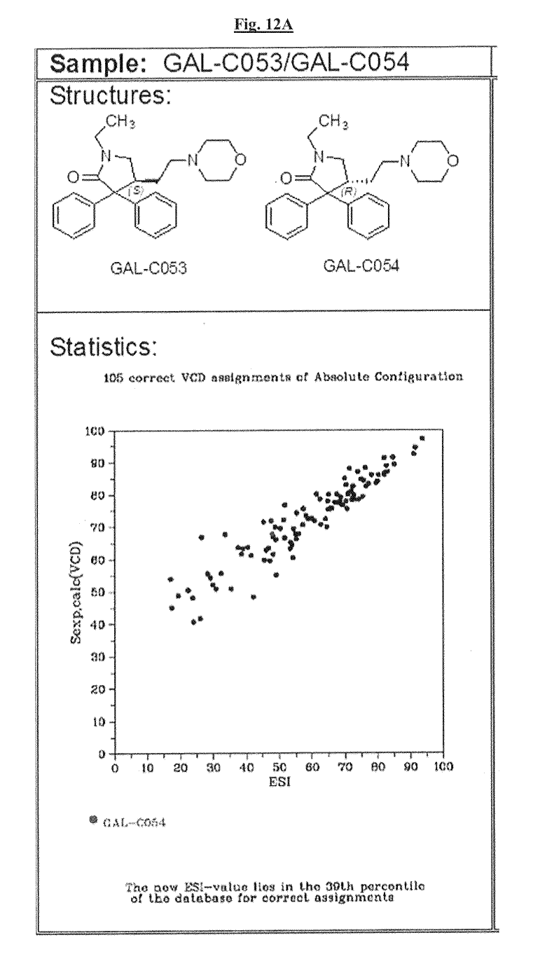

(R)-doxapram was analyzed from 25° C. 300° C., at a rate of 10° C. per minute and was found to have endotherms at 182° C. (FIG. 10),

### X-ray Powder Diffraction

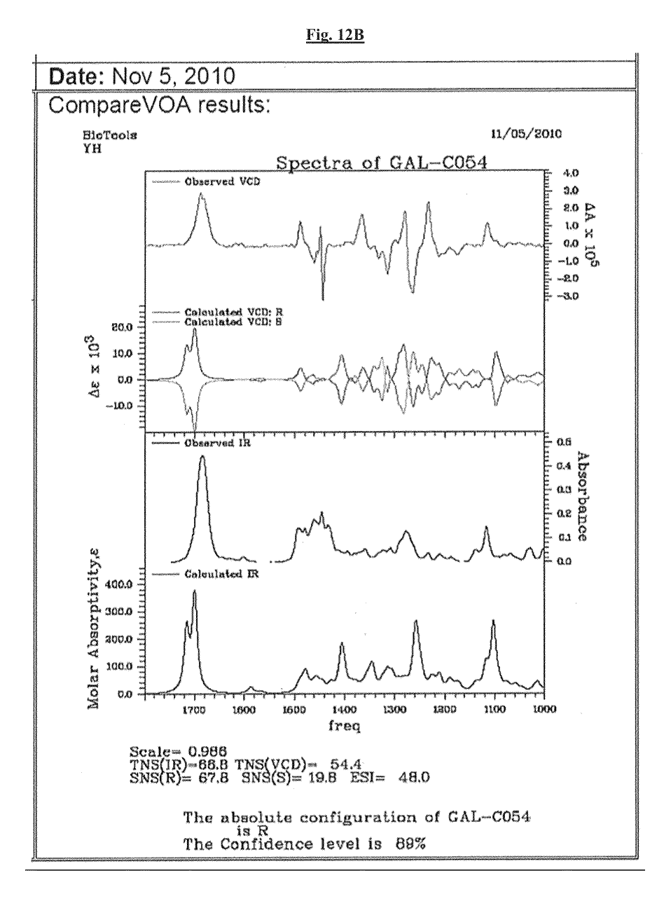

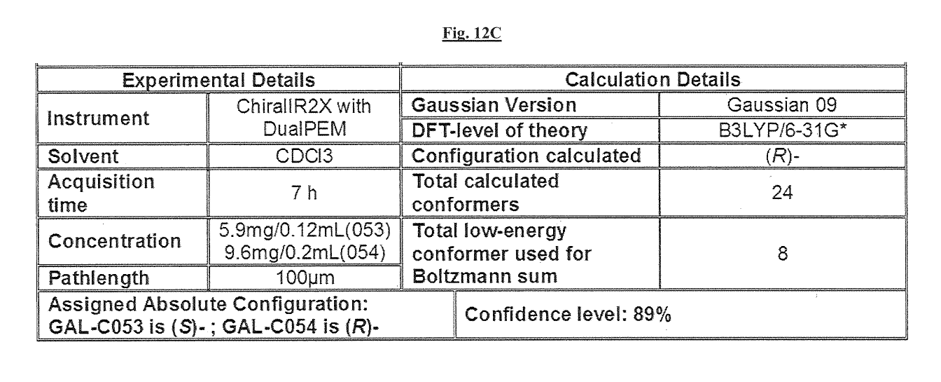

The x-ray diffraction pattern (XPPD) of (R)-doxapram is illustrated in FIG. 11. Table 20 summarizes prominent XRPD Signals for (+)-doxapram hydrochloride.

### Example 4

### Vibrational Circular Dichroism of the (+)-Doxapram and (−)-Doxapram to Assign Absolute Stereochemistry

Vibrational circular dichroism, infrared spectroscopy and optical rotation were used to determine the absolute configuration of (+)-doxapram. Doxapram was separated by chiral column into its two enantiomers as described elsewhere herein.

### Optical Rotation (OR) Measurements

The optical rotation (OR) of (+)-doxapram and (−)-doxapram were measured using a JASCO DIP-370 Polarimeter at 590 nm and 25° C. The measured specific OR values are −88.3°, c=0.8 in EtOH for (−)-doxapram and +10.9°, c=0.9 in EtOH for (+)-doxapram, (+)-Doxapram had appreciable amounts of residual solvent,

### Theoretical Calculations

The (R)-configuration was built with Hyperchem (Hypercube, Inc., Gainesville, Fla.). A conformational search was carried out with Hyperchem for the entire structure at the molecular mechanics level. Geometry optimization, frequency, IR and VCD intensity calculations of the conformers resulting from the conformational search were carried out at the DFT level (B3LYP functional/6-31G (d) basis set) with Gaussian 09 (Gaussian Inc., Wallingford, Conn.). The calculated frequencies were scaled by 0.97 and the IR and VCD intensities were converted to Lorentzian bands with 6-cm⁻¹ half-width at half-height for comparison to experiment.

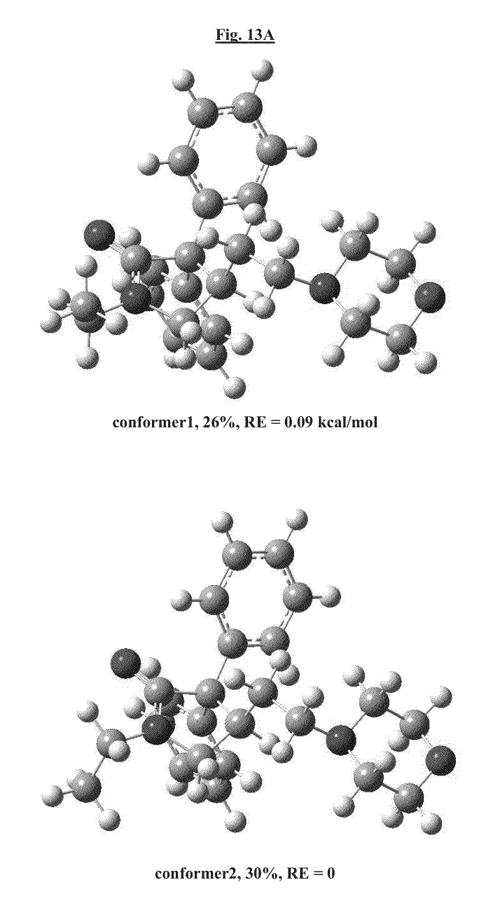

Gaussian calculations identified twelve conformers that had energies within 1.3 kcal/mol from the lowest-energy conformer. The other conformers had energies more than 1.4 kcal/mol higher than the lowest-energy conformer. The optimized geometries of the four lowest-energy conformers of the (R) configuration are illustrated in FIGS. 13A-13B, and the observed VCD and IR spectra along with those of the twelve low-energy conformers are illustrated in FIG. 13C. Based on the overall agreement in VCD pattern for the observed and the Boltzmann sum of the calculated spectra of the twelve lowest-energy conformers (FIG. 13C), the absolute configuration of (+)-doxapram was assigned as (R) and the absolute configuration of (−)-doxapram was assigned as (S).

### Example 5

### Vibrational Circular Dichroism of (+)-Doxapram and (−)-Doxapram to Assign Absolute Stereochemistry

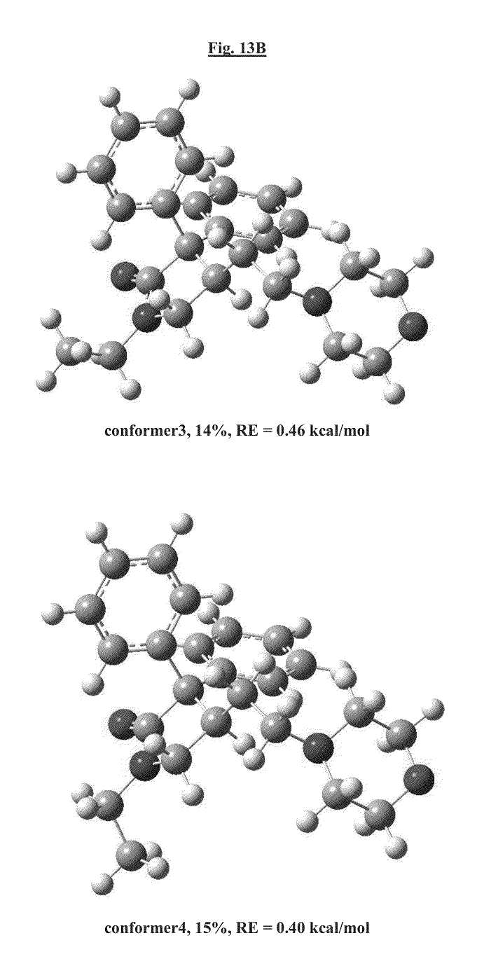

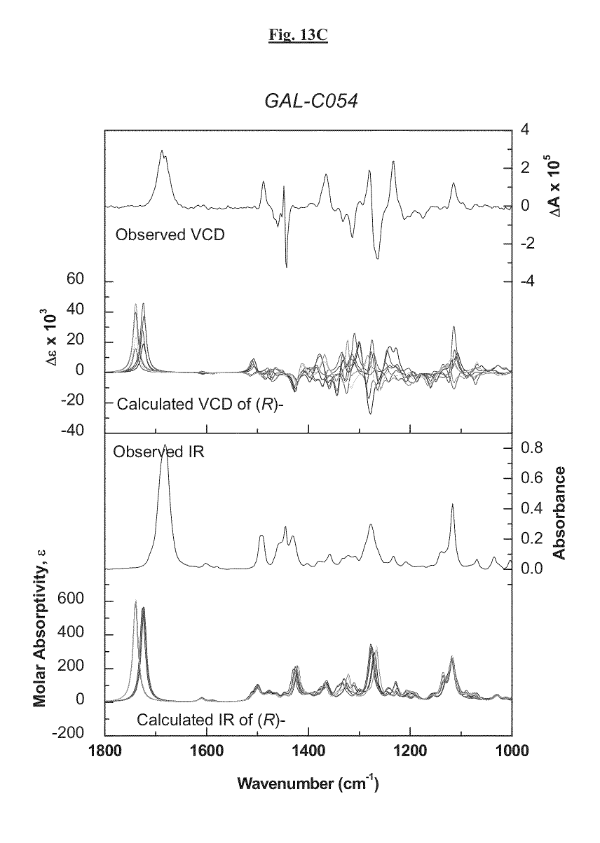

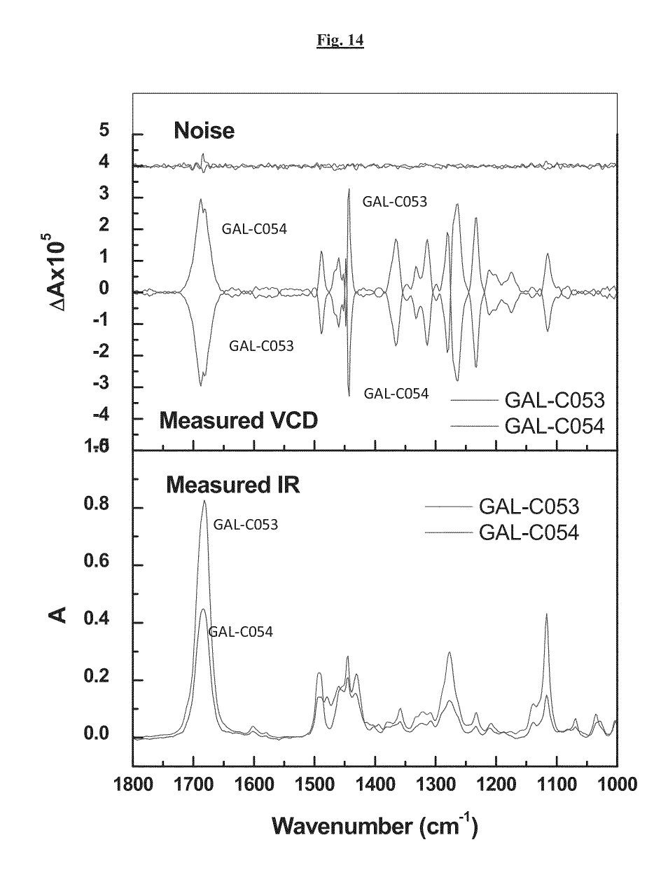

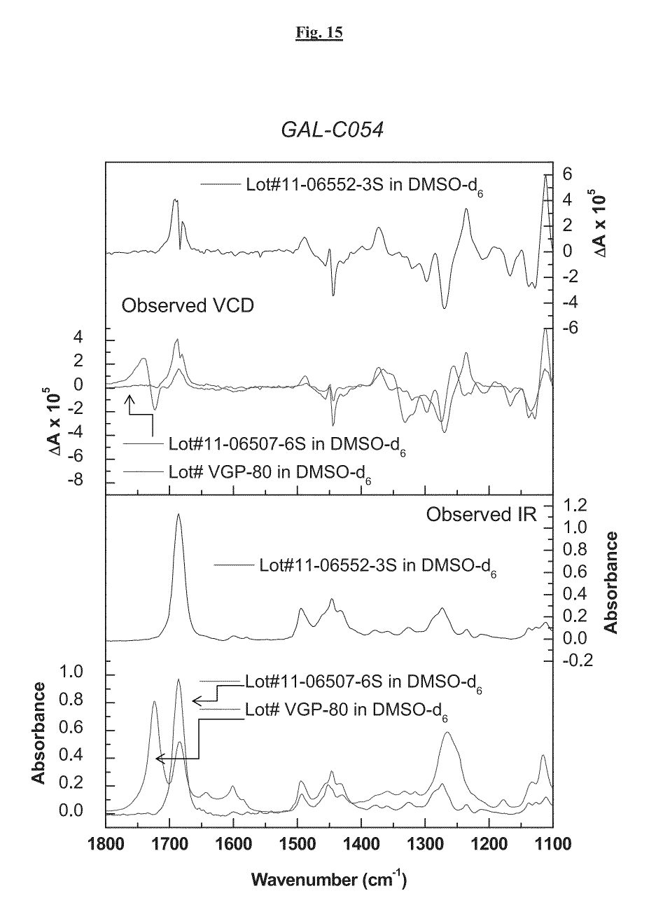

(+)-Doxapram salt was submitted to absolute configuration determination. The absolute configuration of a previous batch of (+)-doxapram free base was determined to be (R) based on comparison of the experimental VCD and IR spectra with those of the calculated (R) configuration (Example 4). The new batch was dissolved in DMSO-d₆ (8 mg/0.13 mL) and placed in a 100 μm pathlength cell with BaF₂ windows. IR and VCD spectra were recorded on a ChiralIR2X® VCD spectrometer (BioTools, Inc.) equipped with dual PEM accessory, with 4 cm⁻¹ resolution, 10 h collection for both (R)-doxapram and DMSO-d₆, and instrument optimized at 1400 cm⁻¹, The solvent-subtracted IR and VCD spectra are compared with those of the previous batches as shown in FIG. 14. The observed VCD features of the new batch were the same as for the previous batch, for which the absolute configuration was determined by comparing the VCD of the freebase with the calculated VCD. Therefore the absolute configuration of the new batch is assigned (R).

### Example 6

### Alternative Chiral Resolution Process to Prepare (−9-Doxapram Hydrochloride Monohydrate Salt

Step 1: Convert (R/S) Doxapram HO to (R/S) Doxapram free base
Step: Preparation of enriched (R)-doxapram (t) e desired enantiomer) from (R/S) Doxapram free base
Step 3: Preparation of (R)-doxapram D-DBTA from enriched (R)-doxapram
Step 3a: Purification of (R)-doxapram D-DBTA Salt (the diastereomeric salt)
Step 4: Preparation of (R)-doxapram HCl from (R)doxapram D-DBTA Salt

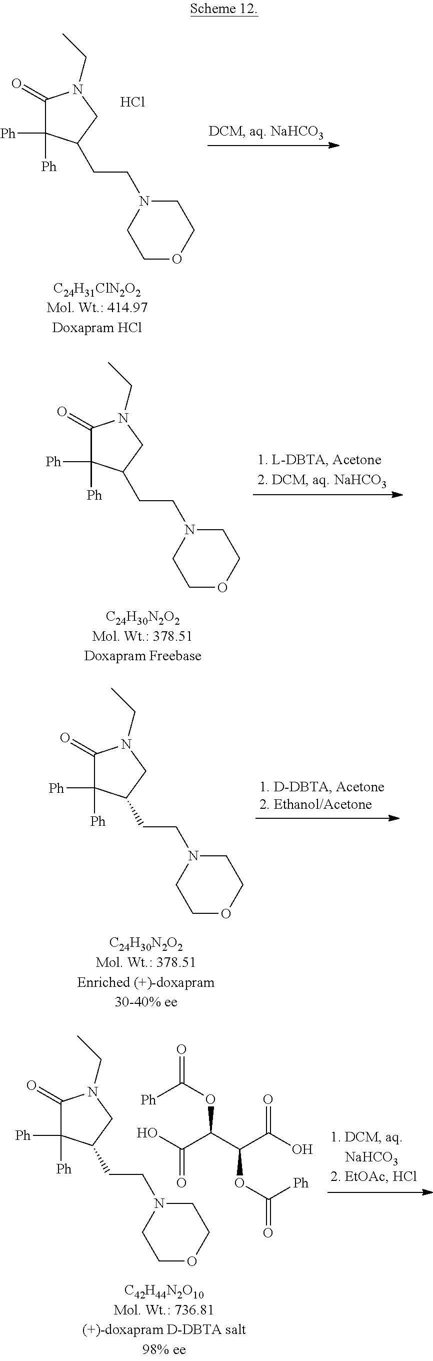

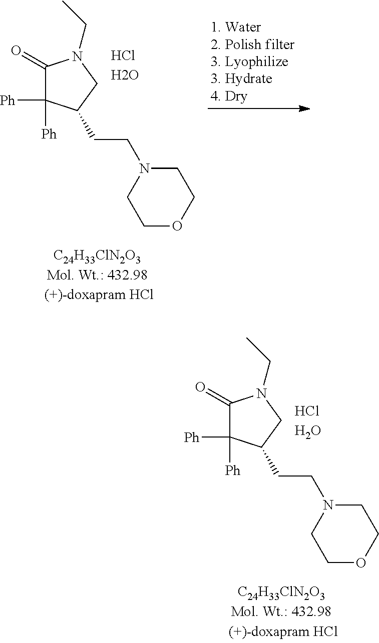

### Step 1: Convert (R/S):Doxapram HO to (R/S) Doxapram Free Base

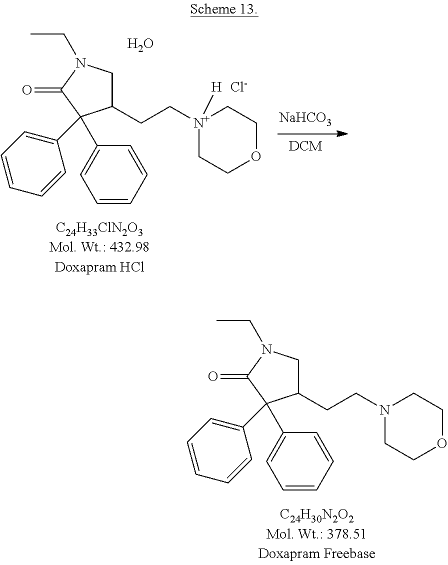

TABLE 12
List of Materials for Step 1.

| | | | | | Actual | Purpose of |
|---|---|---|---|---|---|---|
| Reagents/Materials | MW | Eqs. | Moles | Density | Amt (kg) | Material |
| Doxapram HCl USP | 432.98 | 1.0 | 2.3 | — | 1.0 | Racemic |
| | | | | | | Drug |
| Dichloromethane, ≧99% | 84.93 | — | — | 1.33 | 11 L | Solvent |
| 4.5% Sodium Bicarbonate, | 84.01 | 1.3 | 3.0 | 1 | 5.6 L & 3 kg | Acid |
| aqueous solution | | | | | | neutralizer |
| Sodium Sulfate, anhydrous | 142.04 | — | — | — | 1 kg | Remove |
| | | | | | | water from |
| | | | | | | solvent |

Doxapram hydrochloride was placed in dichloromethane and extracted with the sodium bicarbonate solution [IPC 1]. After separation of the layers, residual water was removed from the dichloromethane layer with anhydrous sodium sulfate. Doxapram free base was isolated by rotary evaporation of the organic solvent [IPC 2].

### Step 1 In-Process Controls:

IPC Test 1: Measure pH of 4.5% sodium bicarbonate solution—pH 8
IPC Test 2: Gross weight of doxapram free base and collect a retain sample—0.9 kg (103% yield)
   Step 2: Preparation of Enriched (+)-Doxapram from Doxapram Free Base

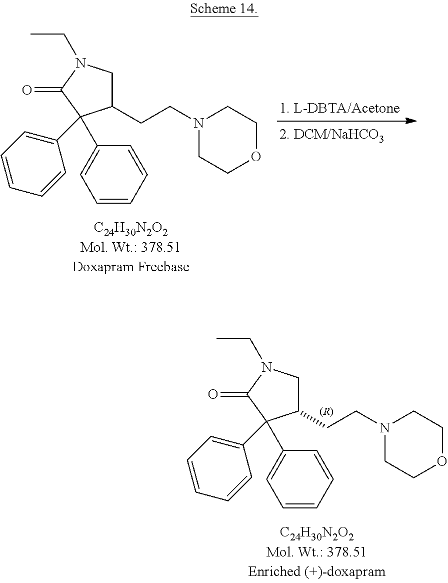

TABLE 13
List of Materials for Step 2.

| | | | | | Actual | Purpose of |
|---|---|---|---|---|---|---|
| Reagents/Materials | MW | Eqs. | Moles | Density | Amt (kg) | Material |
| Doxapram freebase | 378.51 | 1.0 | 2.3 | — | 0.90 | Racemate drug |
| Dibenzoyl-L-tartaric acid, ≧98% | 358.30 | 0.438 | 1.01 | — | 0.36019 | Complex with |
| CAS # 2743-38-6 | | | | | | undesired |
| | | | | | | enantiomer & |
| | | | | | | precipitate from |
| | | | | | | solution |
| Dichloromethane, ≧99% | 84.93 | — | — | 1.33 | 5.6 L | Solvent |
| 9% Sodium Bicarbonate, | 84.01 | 0.68 | — | 1 | 2 L | Break |
| aqueous solution | | | | | | diastereomeric |
| | | | | | | salt to form free |
| | | | | | | base |
| Sodium Sulfate, anhydrous | 142.04 | — | — | — | 1 kg | Remove residual |
| | | | | | | water in solvent |
| Acetone, ≧99% | 58.08 | — | — | 0.79 | 8 L | Solvent |

To a multi-neck RB flask, 0.87 kg of doxapram free base and 6.1 L acetone were added and stirred. To this were added 0.21 kg of L-DBTA at 20° C., and the 5 mixture was stirred for 2 hours. In a separate container, 0.15 kg of L-DBTA were dissolved in 0.2 L of acetone and slowly added to the growing suspension in the multi-neck RB flask over 3 hours. The addition funnel was rinsed with additional acetone. The mixture was agitated for 1 additional hour. The resultant precipitate was filtered off and the cake rinsed with 0.5 L of acetone, Samples of solid and liquid were collected for analysis [IPC 1 & 2]. The filter cake (undesired enantiomer) was vacuum dried and stored for disposal [IPC 6]. The filtrate was concentrated by rotary evaporation. The enriched enantiomer rotovap flasks was dissolved in dichloromethane (2.6 L plus 1 L to rinse flasks) and transferred to a separatory flask. To this solution was slowly added 2 L of 9% sodium bicarbonate solution. The mixture was agitated for 1 hour. If the pH was equal to or greater than pH 8, additional bicarbonate solution was not needed. The organic DOA layer was collected. The separation flask was charged with 1 L of DCM and agitated for at least 10 minutes. The DCM layer was collected. Bicarbonate solution [IPC 3] was collected and assayed for (R)-doxapram content. A 1 kg portion of anhydrous magnesium sulfate was added to the DCM layers, and after agitating the mixture was filtered into flasks for rotary evaporation. The rotovap flasks were rinsed to consolidate the enriched (R)-doxapram free base into one flask. Samples were withdrawn for analysis [IPC 4 & 5].

### Step 2 In-Process Controls—

IPC Test 1: Chiral HPLC of enriched (R)-doxapram in Acetone; (+) 70.56%; (−) 29.44% 41.1% ee
IPC Test 2: Chiral HPLC of S-Doxapram-L-DBTA Salt; (+) 8.4%; (−) 91.6%
IPC Test 3: Achiral HPLC of aqueous bicarbonate layer; Total quantity of S-doxapram and (R)-doxapram was <0.1%.
IPC Test 4: Chiral HPLC of enriched (R)-doxapram (following rotoevaporation);

Chiral HPLC results: (+) 73.7% 26.3% so % ee 47.4%

[Looking to achieve an enrichment of >30%]; Weight 0.55 kg
IPC Test 5: Loss on Drying (105° C. for 2 hours) of Enriched (R)-doxapram (following rotary evaporation); LOD=5.14%
IPC Test 6: Chiral HPLC of S-Doxapram-L-DBTA salt (following vacuum oven drying); 629 g; (+) 19% (−) 81%;

LOD not determined.

### Mass Balance:

Started with 0.9 kg (2.378 moles) of doxapram free base
Ended with 0.522 kg (1.38 moles) of enriched (R)-doxapram free base
[Chiral purity was 73.7% (R)-doxapram so process produced 0.385 kg (1.0 mmoles)]
Yield: 1 mole recovered÷1.19 moles×100=84%
Recovered 0.629 kg of Doxapram-L-DBTA, assuming 5% LOD, 0.6 kg (0.81 moles)
Chiral purity of 19% (R)-doxapram-L-DBTA equals 0.154 moles; lost in cake [IPC 6]
Accountability: 1.19 moles'−1 mole recovered−0.154 mole lost=0.036 mole unaccounted for of (R)-doxapram.
Step 3: Preparation of (R)-Doxapram D-DBTA from Enriched (R)-Doxapram

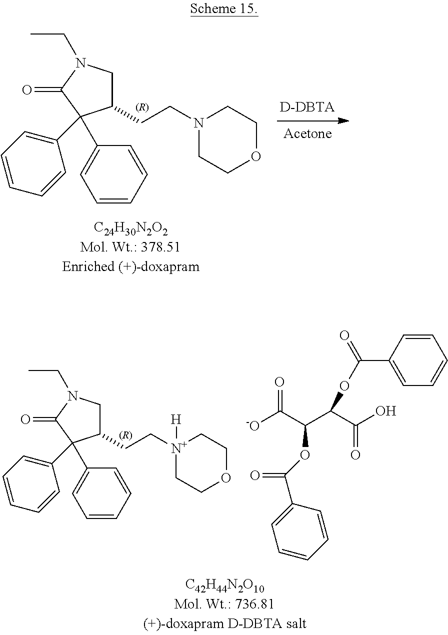

TABLE 14
List of materials for Step 3a.

| | | | | | Actual | |
|---|---|---|---|---|---|---|
| Reagents/Materials | MW | Eqs. | Moles | Density | Amt (kg) | Purpose of Material |
| Enriched (R)-doxapram | 378.8 | 1 | 1.38 | — | 0.522 | Starting material |
| | | | | | Corrected for | containing 2:1-4:1 |
| | | | | | LOD | R to S enantiomer |
| | | | | | | ratio of Doxapram |
| Dibenzoyl-D-tartaric acid, 98% | 358.31 | 0.77 | 1.06 | — | 0.38 | Chiral salt to |
| | | | | | | preferentially |
| | | | | | | complex with R |
| | | | | | | enantiomer |
| Process Water, Filtered | 18.02 | — | — | 1 | 5.3 L | Solvent |
| Acetone, ≧99% | 58.08 | — | — | 0.79 | 3.8 | Solvent |

Into a multineck RB flask, enriched (R)-doxapram (0.6 kg) was dissolved in 3.8 L acetone and stirred. To this solution were added 0.25 eq of D-DBTA, and the mixture was agitated for at least 30 minutes. The quantity of additional D-DBTA to be added was estimated based on the R-doxapram free base (R)-doxapram) content determined in Step 2 IPC 4&5; total D-DBTA added was 0.77 eq (operator discretion). The D-DBTA was dissolved in acetone and added to the RB flask slowly over at least 3 hours. The slurry was filtered and the cake washed with acetone. The filtrate and cake samples were tested [IPC 1 & 2]. The filtrate was agitated for at least 30 minutes in the RB flask to enhance further precipitation. The suspension was filtered and the cake rinsed; and samples were withdrawn from the cake and rinsed for testing [IPC 3 & 4]. The agitation, filtration and rinse process was repeated [IPC 5]. The wet cake was collected, weighed and samples were submitted for analysis [IPC 6].

### Step 3: In-Process Controls—

IPC Test 1: Chiral HPLC of (R)-doxapram-D-DBTA Salt (filter cake 1st); (+) 89.853; (−) 10.1461%, so % ee is 79.7%
IPC Test 2: Chiral HPLC of (R)-doxapram-D-DBTA Salt (loss to 1^{st} filtrate 1st); (+) 21.583%; (−) 78.416% so % ee is 56.8%
IPC Test 3: Chiral HPLC of Acetone filtrate; Sample 16: (+) 20.30%; (−) 79.69%
IPC Test 4: Chiral HPLC of (R)-doxapram-D-DBTA Salt (2^{nd} precipitation); (+) 91.624%; (−) 8.376% so ee % is 83.248%
IPC Test 5: Chiral HPLC of Acetone filtrates; Sample 18: (+) 80.7%; (−) 19.3%
IPC Test 6: Chiral HPLC of (R)-doxapram-D-DBTA salt (wet cake); Net weight 1.8 kg; LOD 23.1%; (—F—) 92.1%; (−) 7.9%

### Step 3a: Purification of (±)Doxapram-D-DBTA Salt

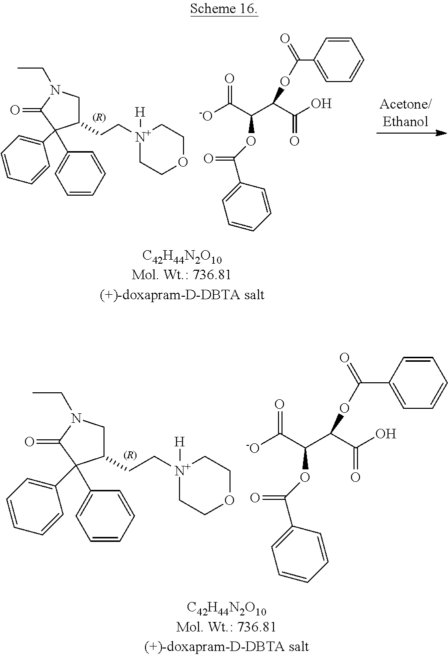

TABLE 15
List of materials for Step 3b.

| | | | | | Expected | Purpose of |
|---|---|---|---|---|---|---|
| Reagents/Materials | MW | Eqs. | Moles | Density | Amt (kg) | Material |
| (R)-doxapram-D-DBTA salt | 736.81 | — | — | — | 1.8 kg** | 60-90% ee pure |
| | | | | | | (R)-doxapram- |
| | | | | | | D-DBTA salt |
| (9v/1v) Acetone (≧99%)/ | — | — | — | — | 8.5 L | Selective |
| Ethanol (≧99%) | | | | | | recrystallization |
| | | | | | | solvent system |

**Charge all wet cake available from previous step

Into a multi-neck RB flask, 8.5 L of acetone/ethanol (9/1 (v/v) and 1.8 kg of (R)-doxapram-D-DBTA (from Step 3) were added, and agitated. The mixture was heated at reflux (approximately 60° C.) for 1 hour. The mixture was cooled to approximately 20° C. and agitated for 1 hour. The content was filtered; the RB flask and cake rinsed with 0.5 L of acetone/ethanol (9/1 (v/v)). Samples of cake and filtrate were collected and submitted for chiral assay [IPC 1 & 2]. The filtrate had a high ratio of the desired enantiomer, so the reflux temperature was reduced to ≧40° C. for additional purification steps. The filtrate was also concentrated to dryness (0.25 kg) and purified separately; see Step 3a third purification. The cake (0.6 kg) did not have an enantiomeric purity ≧98.5% re, and was re-sampled for chiral HPLC analysis. A second purification of this material was necessary.

The 0.6 kg and 0.25 kg of (R)-doxapram-D-DBTA were reprocessed separately using a 40° C. reflux temperature. The IPC of the repeated purifications were within analytical method variability of the desired enantiomeric purity of ≧98.5% ee to proceed to Step 4. The repurification materials were combined,

Step 3a—1^{st} Purification: In-Process Controls
IPC Test 1: Chiral HPLC of (R)-doxapram-D-DBTA Salt (filtrate); (+) 77.3%; (−) 22.7%%. The reflux temperature in this BOP as changed from 60° C. to 40° C. based on the high content of (+)-enantiomer. Product was recovered by rotary evaporation to dryness (0.25 kg) and purified separately in BOP 01GLL03A (below) purification.
IPC Test 2: Chiral HPLC of (R)-doxapram-D-DBTA Salt (loss to 1^{st} filtrate 1st); (085.3%; (−) 14.7% so % ee is 70.6% (R)-enantiomer

Resampled: (+) 94.0%; (−) 6.0% so % ee is 88.0%.
IPC Test 3: Record weigh of wet cake of (R)-doxapram-D-DBTA salt-—0.6 Kg of product
   Step 3a—2^{nd} Purification: In-Process Controls
   0.6 kg of (1-O-doxapram-D-DBTA from above as starting material
IPC Test 1: Chiral HPLC of (R)-doxapram-D-DBTA Salt (filtrate); (+) 65.6%; (−) 34.3%
IPC Test 2: Chiral HPLC of (R)-doxapram-D-DBTA Salt (loss to 1^{st} filtrate 1st); (+) 99.2%; (−) 0.8% so % ee is 98.4%
IPC Test 3: Record weight of wet cake of (R)-doxapram-D-DBTA salt—394.38 g of product
   Step 3a—2^{nd} purification of Step 15 isolated product: In-Process Controls 0.25 kg of (R)-doxapram-D-DBTA from step 15 in 3a 1^{st} purification as the starting material
IPC Test 1: Chiral HPLC of (R)-doxapram-D-DBTA Sail (filtrate); (+) 46.3%; (−) 53.7%%
IPC Test 2: Chiral HPLC of (R)-doxapram-D-DBTA Salt (loss 1^{st} filtrate 1st); (+) 98.9%; (−) 1.1% 1.1% so % ee is 97.8%;
IPC Test 3: Record weigh of wet cake of (R)-doxapram-D-DBTA salt—171.88 g of product
   From 3b purifications, 171.88 g and 394.38 g of (R)-doxapram-D-DBTA were isolated (Total=566.26 g).

### Mass Balance:

Started with 0.522 kg (1.38 moles) of enriched (R)-doxapram free base [1 mole of (R)-doxapram free base]
Ended with two wet cakes totaling 0.56626 kg of (R)-doxapram-L-DBTA [99% chiral purity]
[Chiral purity was 99% (R)-doxapram]
First purification produced: 0.25 kg of 77.3% (R)-doxapram-L-DBTA and 0.6 kg of 94% (R)-doxapram-L-DBTA in wet cake form
Recovered material cannot be calculated because the wet cakes were not dried.
Assuming the cakes were dry powder, the calculated quantity of the R-enantiomer would be 1 mmole.
Step 4: Preparation of (R)-Doxapram Monohydrate Hydrochloride from (R)-Doxapram-D-DBTA Salt

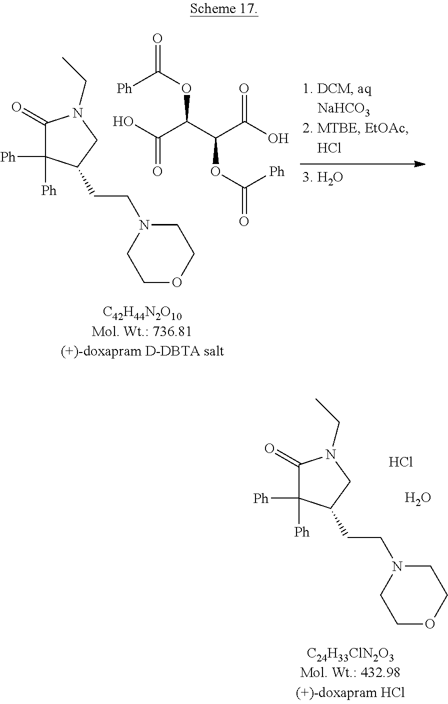

TABLE 16
List of Materials for Step 4.

| | | | | | Actual | |
|---|---|---|---|---|---|---|
| Reagents/Materials | MW | Eqs. | Moles | Density | Amt (kg) | |
| (R)-doxapram-D- | 736.81 | 1 | 0.68 | — | 0.50 | |
| DBTA salt | | | | | | |
| Dichloro- | 84.93 | — | — | 1.33 | 5.9 L & 9 L | |
| methane, ≧99% | | | | | | |
| 4.5% Sodium | 84.01 | — | — | 1 | 6.1 | |
| Bicarbonate, | | | | | | |
| aqueous solution | | | | | | |
| Methyl tert- | 88.15 | — | — | 0.74 | 13.5 L & 6 L | |
| Butyl Ether | | | | | | |
| (MTBE), ≧99% | | | | | | |
| Hydrogen | 36.46 | — | — | — | 0.05 | kg |
| chloride gas | | | | | | |
| Ethyl | 88.1 | — | — | 0.90 | 1 L & 0.8 L | |
| Acetate, ≧99% | | | | | | |
| Water for | 18.02 | — | — | 1 | 1.4 | L |
| Injection, | | | | | | |
| Quality Water | | | | | | |
| Process Water, | 18.02 | — | — | 1 | 5 | L |
| Filtered | | | | | | |

In a separator) flask, 3.5 L of dichloromethane (DCM) and 0.5 kg (R)-doxapram-D-DBTA were added and agitated. To this mixture were slowly added 3.4 L of 4.5% sodium bicarbonate, followed by agitation for 1 hour. The pH was maintained at ≧8 by the additional of 1 L of 4.5% sodium bicarbonate. The organic DCM layer was collected. The aqueous phase was extracted by charging separatory flask with it 0.4 L of DCM and agitating for 10 minutes. The organic DCM layer was collected. The aqueous phase layer was collected. The empty separatory flask was charged with (R)-doxapram in DCM, to which 1.7 L of 4.5% sodium bicarbonate solution were slowly added followed by agitation for 10 minutes. The organic and aqueous layers were collected. The empty separator flask was charged with (R)-doxapram in DCM followed by 1.4 L of water for injection USP which was slowly added. The mixture was agitated for 10 minutes. The two layers were separately collected. The (R)-doxapram in DCM layer was concentrated via, rotary evaporation rinsing the flasks with DCM. To this was added MTBE and the mixture was concentrated by rotary evaporation to remove residual water. This process was repeated several times (to meet IPC1). Material was sampled to determine residual water content [IPC 1] and to determine weight of residual API in RB flask (0.25 kg).

A 3-L multineck RB flask was charged with 1 L of ethyl acetate, cooled to 15° C., and HCl gas was bubbled into the flask to charge with approximately 40 g of HCl gas maintaining the temperature at approximately 15° C. A 25 mL sample of HCl in ethyl acetate was obtained for titration [IPC 2]. The remaining HCl solution was diluted with 800 mL ethyl acetate to prepare a 1.1N HCl solution [IPC 2a].

In a rotovap flask containing (R)-doxapram, 5 L of MTBE was added to effect dissolution. The solution was then transferred to a clean multi-neck RB flask rinsing flasks with 1 L of MTBE. The mixture was agitated. To this was added a solution of 1.1 eq of 1N HCl in ethyl acetate over 40 minute period with continued agitation at 17° C. for 1 hour. An oil formed [IPC 3, 3a, 3b for KF of solutions or solvents] which was concentrated by rotary evaporation to dryness. This material was dissolved in 1.5 L DCM and concentrated via rotary evaporation and stored. The material was dissolved in 3 kg MTBE, passed through a polishing filter (0.2 micron) and the filtrate was concentrated via rotary evaporation.

This procedure was repeated, this time dissolving the material in 1.5 kg MTBE followed by rotary evaporation. This material was subjected to the process once more, this time dissolving in 3 kg MTBE and passing through another polishing filter (0.2 micron) followed by concentration via rotary evaporation to produce a solid. This solid was dried in a vacuum oven on vacuum trays pre-lined with fluorocarbon film. The material was dried to constant weight over four days. The temperature was ramped from 25-35° C. over 2 hrs, 35° C. to 78° C. over 5 h and 78° C. to 100° C. over 1 h, and held at 100° C. for 11 hours before allowing to ramp cool to 50° C. over 5 hours. Samples to monitor residual solvent level and water content after 23 additional hours of drying were obtained [IPC 4 & 5]. Vacuum oven drying continued, ramping from 26° C. to 100° C. over 6 h and maintaining the temperature at 100° C. for 22 h, then cooling for 5 h to 50° C., then continued to cool to 26° C. over 12 hours [IPA 7]. Vacuum oven heating was performed from 25° C. to 100° C. over 3 hours; maintaining temperature until cooling to 50° C. then cool to 25° C.,

At this time, an empty clean vacuum tray was charged with filtered process water, positioned on bottom shelf of vacuum oven. The temperature was maintained between 25 and 30° C. for 4.5 hours [IP 9] and [IP 10].

The following samples were collected:
QC: 10 g; Retain: 2 g; Microbiologic Limits Test: 15 g; NMR: 5 mg; Amber bottle: 267 g; Total: 294 g

### Step 4 In-Process Controls

IPC Test 1: Water determination by Karl Fisher of (R)-doxapram from the rotovap flask and after drying MTBE solution by rotary evaporation; Expected range ≦0.1%. KF result: 0.04%; Net weight of residue API: 0.25 kg
IPC Test 2: Titration of approximate 1N HCl in ethyl acetate; Limits: 0.7 to 1.3N, Result: 1.9N; Resubmitted after dilution; Result: 1.1N HCl
IPC Test 3: Water determination by Karl Fisher of MTBE from (R)-doxapram plus HCl; Unplanned sample: KF result: 1.438%
IPC Test 3a: Also submitted sample of HCl in EA for KF; KF result: 0.286%
IPC Test 3b: Also submitted sample of MTBE used to dissolve (R)-doxaprani freebase for KF; KF result: 0.082%
IPC Test 4: Water determination by Karl Fisher of MTBE from (R)-doxapram HCl; Unplanned sample (early): KF result: 2.3%
IPC Test 5: Preliminary residual solvent analysis of (R)-doxapram HCl (powder in vacuum oven) after 19 h of drying; Limits: MTBE, ethanol, ethyl acetate and acetone limit NMT 1000 ppm each; DCM limit NMT 300 ppm; Results: KF 1.2%; MTBE 4786 ppm, others <100 ppm
IPC Test 6: Water determination by Karl Fisher of MTBE from (R)-doxapram HCl; Planned sample: KF result 1.2%
IPC Test 6a: Achiral HPLC purity of (R)-doxapram; Result: 100.00%
IPC Test 6b: Residual solvents; Results: MTBE 1109 ppm
IPC Test 7: Karl Fisher [sample #01GLL04-01-73]: Results: 4.2%
IPC Test 8: Karl Fisher and MTBE residual tested; Results: KF 2.5% and MTBE 504 Ppm.
IPC Test 9: Karl Fisher, HPLC purity, HPLC chirality, pH and appearance tested; Results: KF:3.429, pH 4.863; Achiral purity 100.0%; Chiral purity 98.8% ee (99.4% (R)-doxapram); appearance: white solid
IPC Test 10: KF test; Result: 4.345%

### Mass Balance:

Started with 0.5 kg (0.68 triples) of wet (R)-doxapram-D-DBTA (99% chiral purity)
Recovered 294 g of (R)-doxapram HCl H₂O containing 4.345% water, equivalent to 281.2 g (R)-doxapram HCl anhydrous (0.6776 moles)
Yield: 0.6776 moles÷1 mole recovered in BOP Step 2=67.8% yield
Accountability: 1 mole−0.6776 moles=0.3224 moles (134 g of (R)-doxapram HCl H₂O) lost of potential drug substance during chiral separation and purification steps 3 and 3a.

TABLE 17
Step 4B, Double Distill Solvents to Remove Impurities: First Distillation

| | | | | | Actual | |
|---|---|---|---|---|---|---|
| | | | | | Amt | Actual Amt |
| Reagents/Materials | MW | Eqs. | Moles | Density | Processed | Produced |
| Dichloromethane, ≧99% | 84.93 | — | — | 1.33 | 10 L | 7.5 L |
| MTBE, ≧99% | 88.15 | — | — | 0.74 | 10 L | 10 L |

TABLE 18
Step 4B, Double Distill Solvents to Remove Impurities: Second Distillation

| | | | | | Actual | |
|---|---|---|---|---|---|---|
| | | | | | Amt | Actual Amt |
| Reagents/Materials | MW | Eqs. | Moles | Density | Processed | Produced |
| Dichloromethane, ≧99% | 84.93 | — | — | 1.33 | 7.5 L | 4.4 L |
| MTBE, ≧99% | 88.15 | — | — | 0.74 | 10 L | 7 L |

DCM was double distilled at a reflux temperature of ≦40° C. MTBE was double distilled at a reflux temperature of ≦55° C.

Step 4C: Rework (R)-Doxapram HCl Monohydrate Via Extraction of Impurities by Aqueous Solution Polish Filtration and MTBE wash. Followed by Free Base Extraction, and Crystallization of (R)-Doxapram HCl from MTBE Solution.

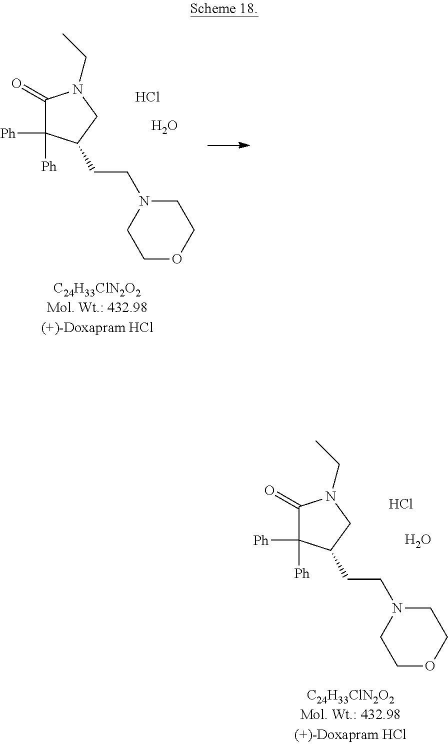 [SMILES: CCN1C[C@H](CCN2CCOCC2)C(c2ccccc2)(c2ccccc2)C1=O ; Cl ; O ; CCN1C[C@H](CCN2CCOCC2)C(c2ccccc2)(c2ccccc2)C1=O ; Cl ; O]

TABLE 19
List of Materials for Step 4A.

| | | | | | Expected | Actual |
|---|---|---|---|---|---|---|
| Reagents/Materials | MW | Eqs. | Moles | Density | Amt | Amt |
| (R)-doxapram HCl | 432.98 | 1.0 | — | — | 140 g | 140 g |
| DCM | 84.93 | | | 1.33 | 4.4 kg | 3.1 kg |
| (double distilled) | | | | | | |
| MTBE | 88.15 | | | 0.74 | 6.9 kg | 6.9 kg |
| (double distilled) | | | | | | |
| Water For Injection | 18.02 | — | — | 1 | 7.7 L | 7.2 L |
| Quality Water, WFI | | | | | | |
| Process Water, Filtered | 18.02 | — | — | 1 | * | * |
| Sodium bicarbonate | 84.01 | | | | 82 g | 82 g |
| HCl gas | 35.46 | | | | 40 g | 150 g |
| Ethyl acetate, ≧99% | 88.1 | | | 0.9 | 0.9 kg | 2.8 kg |
| Sodium Sulfate, | 142.04 | | | | 0.5 | 0.5 |
| anhydrous | | | | | | |

*Not required in this batch

(R)-doxapram HCl H₂O (140 g) was dissolved in 2.8 L of water for injection (WFI), and the solution was passed through a 0.45 μm polypropylene filter. The container was rinsed with an additional 0.5 L of WFI and filtered. In a separator funnel, the combined aqueous phases (filtrates from above) were washed twice with 1.4 of double-distilled MTBE. The container was rinsed with 0.5 L WFI twice. To the aqueous drug solution in a separatory flask obtained from above was then added doubly distilled DCM (1.7 L). Sodium bicarbonate powder (82 g) was added to the mixture and agitated. The aqueous phase was extracted again with 0.9 L DCM (doubly distilled) and the layers collected separately and the separatory flask rinsed with 0.5 L of DCM (doubly distilled). The organic layers were combined and washed with 0.5 L of WFI to remove any trace bicarbonate. Sodium sulfate anhydrous powder (0.5 kg) was added to the organic layer and agitated for 1 hour to reduce water content. This material was then filtered. The organic phase filtrate was evaporated to dryness and then dissolved in 0.5 L MTBE (doubly distilled) and concentrated via rotary evaporation at 50″C. The same procedure was repeated using 0.6 L MTBE (doubly distilled). The (R)-doxapram free base was dissolved in 2 L MTBE (doubly distilled) and passed through a polishing filter to a 3-neck RB flask. The flask was rinsed and transferred with an additional 0.5 L of MTBE (IPC 1).

In a separate flask, 1 L of ethyl acetate was added and HCl gas was bubbled into the solvent. Once 0.1 kg was registered on the scale, the HCl addition was stopped (IPC2). Based on the HCl concentration, the ethyl acetate solution was diluted to approximately 1N HCl and sampled again for MI concentration determination (IPC3).

This solution, of (R)-doxapram free base was cooled in MTBE to ≦15° C. (approximately 10° C.) and 0.277 L of 1.4N HCl in ethyl acetate were added over a 70 minute interval Crystalline-looking precipitate was then collected on a 0.45 um PP filter. No MTBE rinse was used. The wet powder was spread on a vacuum oven lined tray, weighed and dried at 45° C. (50° C.) for 2 hours, then 72° C. for 2 hours, and finally 98° C. for 24 hours (IPC 4 & 5). The residual solvent and water content was within specification so no further drying was performed and hydration was not necessary. Collected samples—10 g for QC release, 2 g for retain, 5 mg for NMR and 105 g for client (stored in amber jar); total 117 g. Label and store amber glass bottle at −20° C.

### Step 4C In-Process Controls—

IPC Test 1: Water determination by Karl Fisher; result: 0.42%
IPC Test 2: HCl concentration in EA; Target approximately 1N HCl solution; results: 3.2N HCl
IPC Test 3: HCl concentration in EA; Target approximately 1N HCl solution; results: 1.3N HCl
IPC Test 4: Residual solvent levels:

| Solvent | Spec limit | Actual value | |
|---|---|---|---|
| Ethyl acetate | ≦1000 ppm | <101 | ppm |
| DCM: | ≦300 ppm | <89 | ppm |
| Acetone: | ≦1000 ppm | <96 | ppm |
| MTBE: | ≦1000 ppm | 66 1 | ppm |

IPC Test 5: Water determination by Karl Fisher of (R)-doxapram HCl H₂O; result: 4.2% (consistent with monohydrated salt)

### Yield: 83.6%

### Example 7

### Carbocyclization Synthesis of Doxapram

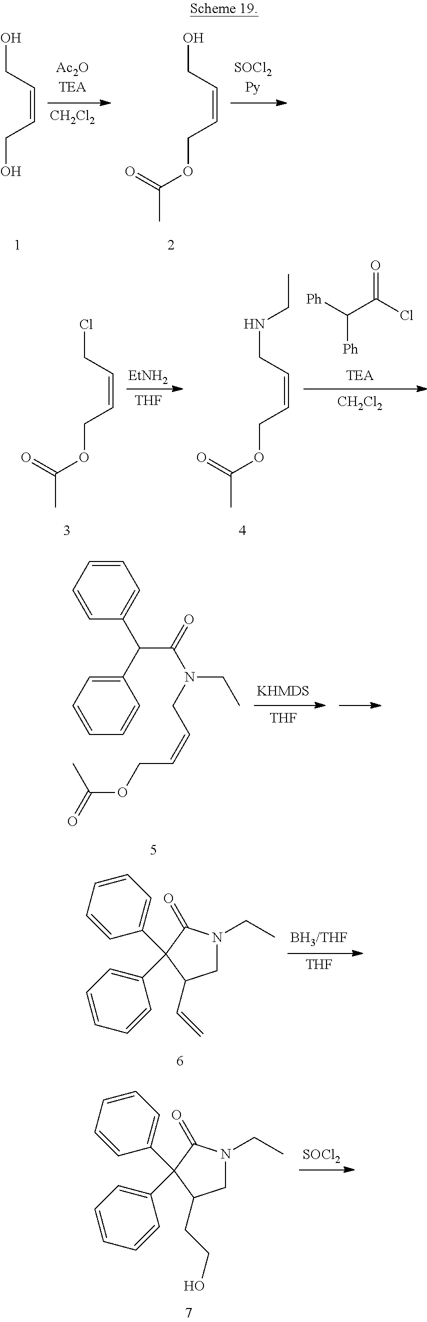

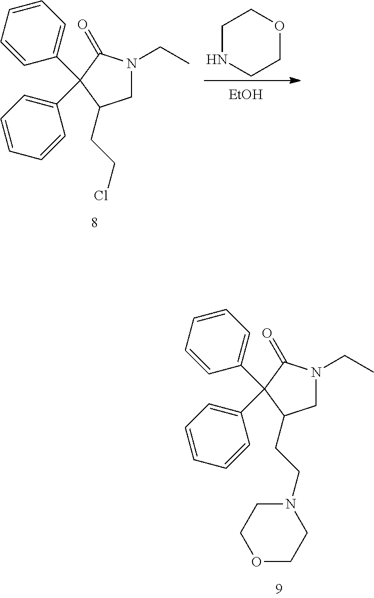

### Acetic acid (Z)-4-hydroxy-but-2-enyl ester (2)

To a solution of (Z)-butene-1.4 diol (1) (10.00 g, 113.49 minor) and TEA (15.8 mL, 113.49 mmol) in CH₂Cl₂ (60 mL) was added Ac₂O (10.7 mL, 113.49 mmol) in dropwise manner. The reaction mixture was stirred at room temperature for 20 h. The solvent was then removed under reduced pressure, and the crude product was suspended in H₂O (30 mL). The suspension was extracted with petroleum ether (3×40 mL) to remove diacetate by-product. The aqueous solution was then extracted with CH₂Cl₂ (4×30 mL).

The combined organic extracts were washed with saturated NaHCO₃ solution and dried over anhydrous Na₂SO₄, and then filtered and evaporated to yield acetic acid (Z)-4-hydroxy-but-2-enyl ester (2) (6.70 g, 45%). 400 MHz ¹H NMR (CDCl₃, ppm) 5.88-5.80 (1H, m) 5.65-5.58 (1H, m) 4.67-4.64 (2H, m) 4.26-4.23 (2H, m) 2.06 (3H, s). Rf=0.35 (petroleum ether/EtOAc: 1/1 (v/v)).

### Acetic acid (Z)-4-chloro-but-2-enyl ester (3)

The solution of acetic acid, (Z)-4-hydroxy-but-2-enyl ester (2) (6.00 g, 46.10 mmol) in pyridine (21.4 mL, 276.62 mmol) was cooled to 0° C. After cooling, SOCl₂ (20.2 mL, 276.62 mmol) was added in a dropwise manner. The reaction mixture was stirred at room temperature for 4 h. The mixture then was cooled to 0° C. (ice bath) and then slowly treated via the addition of a 50% K₇CO₃ solution (˜250 mL) until the vigorous destruction of excess thionyl chloride is completed. Then the resulting mixture was extracted with CH₂Cl₂ (3×100 mL). The combined organic extracts were washed with 1N HCl (2×200 mL) and dried over anhydrous Na₂SO₄. After filtration, the solvent was evaporated and the residue was distilled at 1 mm Hg pressure and 80° C. to afford acetic acid (Z)-4-chloro-but-2-enyl ester (3) (3.00 g, 44%). 400 MHz ¹H NMR (CDCl₃, ppm) 5.89-5.79 (1H, m) 5.76-5.68 (1H, m) 4.67-4.64 (2H, m) 4.13-4.11 (2H, m) 2.06 (3H, s). Rf=0.58 (petroleum ether/EtOAc: 3/1 (v/v)). GC-MS (m/z): 113 (M−Cl).

### Acetic acid (Z)-4-ethylamino-but-2-enyl ester (4)

Acetic acid (Z)-4-chloro-but-2-enyl ester (3) (4.37 g, 29.41 mmol) was added to a cooled (0° C.) ethylamine solution in THF (2M, 30 ml) under an argon atmosphere. The vial was closed and the mixture heated at 50° C. for 24 h. The volatiles were then removed under reduced pressure, and 30% K₂CO₃ solution (80 mL) was added. The resulting suspension was extracted with CH₂Cl₂ (4×60 mL). The combined organic extracts were dried over anhydrous Na₂SO₄, filtered, and evaporated. The resultant residue was distilled at 1 mm Hg pressure and 40° C. to yield acetic acid (Z)-4-ethylamino-but-2-enyl ester (4) (1.49 g, 32%). 400 MHz ¹H NMR (CDCl₃, ppm) 5.78-5.70 (1H, m) 5.66-5.58 (1H, m) 4.65-4.62 (2H, m) 3.33 (2H, dd, J=6.8, 1.2 Hz) 2.65 (2H, q, J=7.2 Hz) 2.05 (3H, s) 1.11 (3H, 1, J=7.2 Hz). Rf=0.23 (CH₂Cl₂/MeOH-9/1). ESI-MS (m/z): 158 (M+H)⁺.

### Acetic acid (Z)-4-(diphenylacetyl-ethyl-amino)-but-2-enyl ester (5)

Triethylamine (1.1 mL, 8.15 mmol) and diphenylacetyl chloride (1.87 g. 8.11 mmol) was added dropwise to a cooled (0° C.) solution of acetic acid (Z)-4-ethylamino-but-2-enyl ester (4) (1.07 g, 6.81 mmol) in dry CH₂Cl₂ (20 mL) under an argon atmosphere. The reaction mixture was stirred at room temperature for 6 h. After this time, water (40 mL) was added and the resulting suspension was extracted with CH₂Cl₂ (3×20 mL). The combined organic extracts were washed with water (80 mL) and dried over anhydrous Na₂SO₄. The solvents were evaporated and the crude product was purified by flash chromatography [gradient elution from petroleum ether/EtOAc: 9.1 (v/v) to petroleum ether/EtOAc:2:1 (v/v)], followed by reverse phase chromatography [gradient elution from MeCN/H₂O: 1:9 (v/v) to MeCN/H₂O: 99:1 (v/v)] to yield acetic acid (Z)-4-(diphenylacetyl-ethyl-amino)-but-2-enyl ester (5) (1.40 g, 59%). 400 MHz ¹H NMR (CDCl₃, ppm): 7.34-7.21 (10H, m) 5.20-5.17 (0.6H, m) 5.15-5.13 (0.4H, m) 469-4.66 (1.2H, m) 4.60-4.67 (0.8H, m) 4.16-4.11 (1.2H, m) 3.99-3.95 (0.8H, m) 3.45 (0.8H, q, J=7.2 Hz) 3.33 (1.2H, q, J=7.2 Hz) 2.06 (1.2H, s) 2.05 (1.8H, s) 1.15 (1.8H, t, J=7.2 Hz) 1.14 (1.2H, t, J=7.2 Hz) as rotamer mixture 40/60. Rf=0.50 (petroleum ether/EtOAc: 3/1 v/v)). ESI-MS (m/z): 352 (M+H)⁺.

### 1-Ethyl-3,3-diphenyl-4-vinyl-pyrrolidin-2-one (6)

An oven dried vial was charged acetic acid (Z)-4-(diphenylacetyl-ethyl-amino)-but-2-enyl ester (5) (0.53 g, 1.51 mmol) and THF (6 mL). The vial was cooled to −78° C. (dry CO₂/acetone bath) and a KHMDS solution in THF (0.91 M, 1.80 mL, 1.64 mmol) was added in a dropwise manner under an argon atmosphere. The reaction mixture was stirred at room temperature for 1 h. The solution was treated with saturated NH₄Cl solution (20 mL) and then extracted with EtOAc (3×15 mL). The combined organic extracts were washed with H₂O (50 mL), then with a brine solution (40 mL) and dried over anhydrous Na₂SO₄. The volatiles were removed via evaporation and the crude product was purified by flash chromatography (gradient elution from petroleum ether/EtOAc: 9:1 (v/v) to petroleum ether/EtOAc 2:1 (v/v)) to yield 1-ethyl-3,3-diphenyl-4-vinyl-pyrrolidin-2-one (6) (0.30 g, 69%). 400 MHz ¹H NMR (CDCl₃, ppm) 7.58-7.54 (2H, m) 7.34-7.28 (2H, m) 7.27-7.15 (4H, m) 6.96-6.92 (2H, m) 5.40 (1H, ddd, J=17.2, 10.2, 8.4 Hz) 5.22 (1H, ddd, J=17.2, 1.6, 0.8 Hz) 5.05 (1H, ddd, J=10.2, 1.6, 0.8 Hz) 3.86-3.79 (1H, m) 3.61-3.51 (1H, m) 3.50-3.39 (2H, m) 3.21-3.15 (1H, m) 1.21 (3H, t, J=7.4 Hz). ¹³C NMR (100 MHz, CDCl₃): δ=174.4, 141.9, 140.6, 135.9, 129.1, 128.6, 127.9, 127.8, 126.9, 126.7, 117.7, 60.6, 48.3, 46.2, 37.7, 12.5. Rf=0.46 (petroleum ether/EtOAc-3/1). ESI-MS (m/z): 292 (M+H)⁺.

### 1-Ethyl-4-(2-hydroxy-ethyl-3,3-diphenylpyrrolidin-2-one (7)

A BH₃ in THF solution (1 M, 1.02 mL) was added to the solution of 1-ethyl-3,3-diphenyl-4-vinyl-pyrrolidin-2-one (6) (150 mg, 0.51 mmol) in dry THF (5 mL) at 0° C. under an argon atmosphere. The reaction mixture was stirred at room temperature for 4 h, then cooled to 0° C. and treated via, the sequential additions of 4 N NaOH (3 mL) and 35% H₂O₂ (3 ml). The mixture was stirred at room temperature for 2 h and then saturated NH₄Cl solution (10 mL) was added. The resultant suspension was extracted with CH₂Cl₂ (3×10 mL). The combined organic extracts were washed with saturated NH₄Cl solution (10 mL) and dried over anhydrous Na₂SO₄. The volatiles were evaporated and the crude product was purified by flash chromatography (gradient elution from petroleum ether/EtOAc: 4:1 (v/v) to petroleum ether/EtOAc: 1:9 (v/v)) to yield 1-ethyl-4-(2-hydroxy-ethyl)-3,3-diphenyl-pyrrolidin-2-one (7) (70 mg, 45%). 400 MHz ¹H NMR (CDCl₃, ppm) 7.57-7.52 (2H, m) 7.36-7.30 (2H, m) 7.30-7.17 (4H, m) 6.92-6.87 (2H, m) 3.75-3.62 (2H, m) 3.62-3.52 (2H, m) 3.49-3.37 (2H, m) 3.10-3.04 (1H, m) 1.81-1.71 (1H, m) 1.55-1.44 (1H, m) 1.23 (3H, t, J=7.2 Hz) 1.00-0.89 (1H, m). Rf=0.30 (petroleum ether/EtOAc: 1:1 (v/v)) ESI-MS (m/z): 310 (M+H)⁺.

### 4-(2-Chloro-ethyl)-1-ethyl-3,3-diphenyl-pyrrolidin-2-one (8)

A mixture of 1-ethyl-4-(2-hydroxy-ethyl)-3,3-diphenyl-pyrrolidin-2-one (7) (70 mg, 0.22 mmol) and SOCl₂ (0.5 mL) was heated at reflux for 5 h. The mixture was cooled to 0° C. and treated with 50% aqueous K₂CO₃ solution (˜20 mL) until the vigorous reaction with thionyl chloride is ceased. The mixture was then extracted with CH₂Cl₂ (3×10 mL), and the combined organic extracts were washed with saturated NH₄Cl solution (20 mL) and dried over anhydrous Na₂SO₄. The solvents were evaporated and the crude product was purified by flash chromatography (gradient elution from petroleum ether/EtOAc: 9:1 (v/v) to petroleum ether/EtOAc: 2:1 (v/v)) to yield 4-(2-chloro-ethyl)-1-ethyl-3,3-diphenyl-pyrrolidin-2-one (8) (70 mg, 97%). 400 MHz ¹H NMR (CDCl₃, ppm) 7.57-7.51 (2H, m) 7.37-7.31 (2H, m) 7.30-7.17 (4H, m) 6.90-6.85 (2H, m) 3.63-3.49 (4H, m) 3.49-3.39 (2H, m) 3.03 (1H, t, J=8.2 Hz) 2.00-1.61 (1H, m) 1.23 (3H, t, J=7.2 Hz) 1.21-1.12 (1H, m). ¹³C NMR (100 MHz, CDCl₃): δ=174.6, 141.1, 141.0, 128.7, 128.6, 128.1, 127.9 127.1, 126.9, 60.1, 47.9, 42.9, 37.8, 37.5, 32.8, 12.6. Rf=0.50 (petroleum ether/EtOAc-3/1). ESI-MS (m/z): 328, 330 (M+H)⁺.

### 1-Ethyl-4-(2-morpholin-4-yl-ethyl)-3,3-diphenyl-pyrrolidin-2-one (9)

A mixture of 4-(2-chloro-ethyl)-1-ethyl-3,3-diphenyl-pyrrolidin-2-one (8) (67 mg, 0.20 mmol) and morpholine (72 μL, 0.81 mmol) EtOH (2 mL) was heated in a dosed vial at 105° C. for 16 h. The volatiles were removed in vacuo and water (10 mL) was added. The resulting suspension was extracted with EtOAc (3×10 mL). The combined organic extracts were washed with water (2×10 mL), then with a brine solution (10 mL) and lastly, dried over anhydrous Na₂SO₄. The crude product was purified by flash column chromatography [gradient elution from EtOAc to EtOAc/MeOH: 80/20 (v/v)] to yield 1-ethyl-4-(2-morpholin-4-yl-ethyl)-3,3-diphenyl-pyrrolidin-2-one (9) (52 mg, 69%). 400 MHz ¹H NMR (CDCl₃, ppm) 7.55-7.51 (2H, m) 7.36-7.30 m) 7.29-7.17 (4H, m) 6.92-6.87 (2H, m) 3.72-3.68 (4H, m) 3.64-3.51 (2H, m) 3.48-3.38 (1H, m) 3.35-3.26 (1H, m) 104 (1H, dd, J=9.0, 9.0 Hz) 144-126 (6H, m) 1.71-1.62 (1H, m) 1.23 (3H, t, J=7.2 Hz) 0.92-0.80 (1H, m) ESI-MS (m/z): 3.79 [M+H]⁺.

### Example 8

### Asymmetric Synthesis of (+)-Doxapram

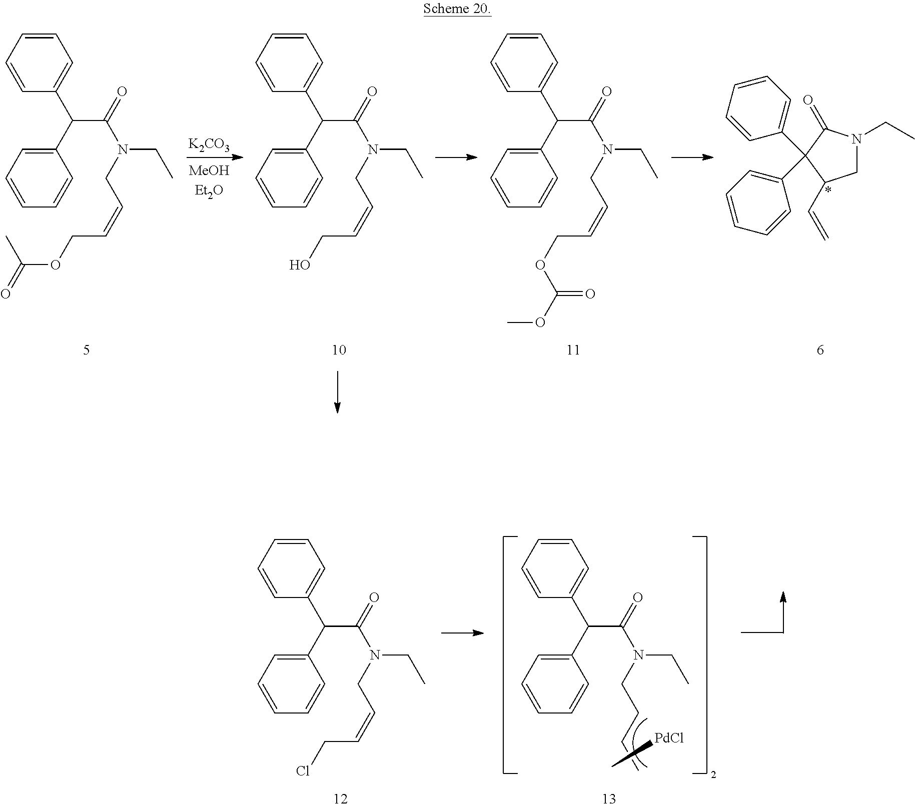

### (Z)—N-ethyl-N-(4-hydroxybut-2-enyl)-2,2-diphenylacetamide (10)

A mixture of acetic acid (Z)-4-(diphenylacetyl-ethyl-amino)-hut-2-enyl ester (5) (520 mg, 1.48 mmol) and K₂CO₃ (20 mg, 0.15 mmol) in MeOH (3 mL) and Et₂O (3 mL) was stirred at room temperature for 1 h. Water (10 mL) was added and the resulting suspension was extracted with CH₂Cl₂ (3×10 mL). The combined organic extracts were washed with water (20 mL) and dried over anhydrous Na₂SO₄. The volatiles were removed in vacuo to yield (Z)—N-ethyl-N-(4-hydroxybut-2-enyl)-2,2-diphenylacetamide (10) (420 mg, 92%) as a rotamer mixture (30/70). 400 MHz ¹H NMR (CDCl₃, ppm) 7.27-7.21 (4H, m) 7.20-7.14 (6H, m) 5.86-5.78 (0.7H, m) 5.75-5.66 (0.3H, m) 5.49-5.35 (1H, m) 5.09 (1H, s) 4.18-4.10 (2H, m) 4.01 (1.4H, d, J=7.8 Hz) 3.87 (0.6H, d, J=6.6 Hz) 3.39 (0.6H, q, J=7.2 Hz) 3.31 (1.4H, (4, J=7.2 Hz) 3.17-3.04 (1H, s) 1.12 (1.4H, t, J=7.2 Hz) 1.08 (0.611, t, J=7.2 Hz). Rf=0.18 (petroleum ether/EtOAc 3:1). ESI-MS (m/z): 310 [M+H]⁺.

### (Z)-4-(N-ethyl-2,2-diphenylacetamido)but-2-enyl methyl carbonate (11)

To a mixture of (Z)—N-ethyl-N-(4-hydroxybut-2-enyl)-2,2-diphenylacetamide (10) (700 mg, 2.26 mmol), DMAP (28 mg g, 0.23 mmol) and TEA (0.94 mL, 6.79 mmol) in CH₂Cl₂ (6 mL), methyl chloroformate (0.35 mL, 4.52 mL) was added in a dropwise manner at 0° C. The resultant mixture was stirred at 0° C. for 1 h after which time, saturated NH₄Cl solution (15 mL) was added. The resulting suspension was extracted with CH₂Cl₂ (3×15 mL). The combined organic extracts were washed with saturated NaHCO₃ solution (20 mL), then with water (20 mL) and lastly, dried over anhydrous Na₂SO₄. The volatiles were removed and the crude product was purified by flash column chromatography (PE:EtOAc: 4:1 (v/v) as eluent) to yield (Z)-4-(N-ethyl-2,2-diphenylacetamido)but-2-enyl methyl carbonate (It) (450 mg, 54%) as mixture of rotamers (30/70). 400 MHz ¹H NMR (CDCl₃, ppm) 7.32-7.19 (10H, m) 5.76-5.60 (1.6H, m) 5.56-5.49 (0.4H, m) 5.17 (0.6H, s) 5.13 (0.4H, s) 4.74 (1H, d, J=6.6 Hz) 4.62 d, J=7.7 Hz) 4.12 (1H, d, J=6.6 Hz) 3.97 (1H, d, J=6.6 Hz) 3.78 (1H, s) 3.75 (2H, s) 3.44 (1H, q, J=7.2 Hz) 3.32 (1H, q, J=7.2 Hz) 1.13 (3H, t, J=7.2 Hz). Rf=0.5 (PE/EtOAc-3:1). ESI-MS (m/z): 368 [M+H]⁺.

### Enantiomerically Enriched 1-Ethyl-3,3-diphenyl-4-vinyl-pyrrolidin-2-one (6)

An oven dried vial was charged with (Z)-4-(N-ethyl-2,2-diphenylacetamido)but-2-enyl methyl carbonate (11) (33 mg, 0.09 mmol), (−)-sparteine (23 μL, 0.1 mmol) and toluene (1 mL), purged with argon and cooled to −78° C. To this mixture was added 1M LiHMDS in toluene (100 μL, 0.1 mmol) in a dropwise manner at −78° C. The reaction mixture was stirred at ambient temperature for 1 h, and volatiles were removed in vacuo. The mixture was purified using preparative TLC (petroleum ether/EtOAc: 3:1 (v/v) as eluent) to yield enantiomerically enriched 1-ethyl-3,3-diphenyl-4-vinyl-pyrrolidin-2-one (6) (18 trig, 70%) that was determined by chiral HPLC to have an enantiomeric excess of 31%.

### (Z)—N-(4-chlorobut-2-enyl)-N-ethyl-2,2-diphenylacetamide (12)

To a mixture of (Z)—N-ethyl-N-(4-hydroxybut-2-enyl)-2,2-diphenylacetamide (10) (450 mg, 1.45 mmol), DMAP (18 mg, 0.15 mmol), TEA (0.61 mL, 4.36 mmol) and NaCl (255 mg, 4.36 mmol) in CH₂Cl₂ (8 mL), was added mesyl chloride (0.34 mL, 4.36 mL) under an argon atmosphere. The mixture was stirred at ambient temperature for 20 h. After this time, saturated NH₄Cl solution (15 mL) was added, and the resulting suspension was extracted with CH₂Cl₂ (3×15 mL). The combined organic extracts were washed water (20 mL) and dried over Na₂SO₄. The volatiles were removed in vacuo and the crude product was purified by flash column chromatography (PE:EtOAc: 4:1 (v/v) as eluent) to yield (Z)—N-(4-chlorobut-2-enyl)-N-ethyl-2,2-diphenylacetamide (12) (340 mg, 71%) as mixture of rotamers (30/70). 400 MHz ¹H NMR (CDCl₃, ppm) 7.32-7.19 (10H, m, overlapped with CDCl₃) 5.86-5.75 (1H, m) 5.64-5.47 (1H, m) 5.17 (0.6H, s) 5.11 (0.4H, s) 4.15 (1H, d, J=8.0 Hz) 4.11 (1H, dd, J=7.0, 1.2 Hz) 4.01 (1H, d, J=8.0 Hz) 3.94 (1H, dd, J=6.4, 1.4 Hz) 3.45 (1H, q, J=7.2 Hz) 3.32 (1H, q, J=7.2 Hz) 1.14 (1.4H, t, J=7.2 Hz) 1.13 (0.6H, t, J=7.2 Hz). Rf=0.64 (PE/EtOAc-3:1). ESI-MS (m/z): 328 (M+14)⁺.

### bis(η³-1-(Ethyl-(diphenylacetyl)aminomethylyl)-allyl)-dichloro-dipalladium(II)(13)

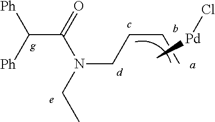

An oven dried flask was charged with tris(dibenzylideneacetone) dipalladium(0) (Pd₂ dba₃) (838 mg, 0.92 mmol), THF (6 mL) and (Z)—N-(4-chlorobut-2-enyl)-N-ethyl-2,2-diphenylacetamide (12) (600 mg, 1.83 mmol) in acetonitrile (3 mL). Additional THF (4 mL) was added under argon atmosphere. The resulting reaction mixture was stirred at ambient temperature for 1 h as the color of the reaction mixture changed from violet to green. The volatiles were removed and the resulting crude mixture was purified by flash column chromatography (PE:EtOAc: 2:1 (v/v) as eluent) to yield bis(η³-1-(ethyl-(diphenylacetyl)aminomethylyl)-allyl)-dichloro-dipalladium(II) (13) (616 mg, 71%) as yellow powder. 4 00 MHz ¹H NMR (CDCl₃, ppm) 736-7.16 (10H, m) 5.45-5.46 (1H, m, Hb) 5.17 (1H, s, Hg) 3.91 (1H, d, J=6.8 Hz, Ha anti) 3.75-3.30 (5H, m, Hc, Hd, He) 2.90 (1H, d, J=13.0 Hz, Ha anti) 1.11 (3H, t, J=7.0 Hz). Rf=0.29 (PE/ETOAc:3:1 (v/v). ESI-MS (m/z): 439 (M+MeCN)⁺, 911 (M+2MeCN+MeOH)⁻.

### 1-Ethyl-3,3-diphenyl-4-vinyl-pyrrolidin-2-one (6)

An oven dried vial was charged with palladium complex 13 (100 mg, 0.115 mmol), R-B1NAP (143 mg, 0.23 mmol) and DMF (2 mL) and purged with argon. After cooling to −30° C., KHMDS in THF (0.911M, 0.25 mL, 0.23 mmol) was added. The mixture was stirred at room temperature for 2 h. After this time, saturated NH₄Cl solution (10 mL) was added, and the resulting suspension was extracted with EtOAc (3×10 mL). The combined organic extracts were washed with water (20 mL), then with a brine solution (20 mL), and dried over anhydrous Na₂SO₄. The crude product was purified by flash column chromatography (PE:EtOAc:4:1 (v/v) as eluent) to yield (R)-enantiomerically enriched ethyl-3,3-diphenyl-4-vinyl-pyrrolidin-7-one (6) (30 mg, 45%) that was determined by chiral HPLC to have an enantiomeric excess of 70%.

### Example 9

### Chiral Synthesis of (+)-Doxapram via of (S)-3-hydroxypyrrolidine

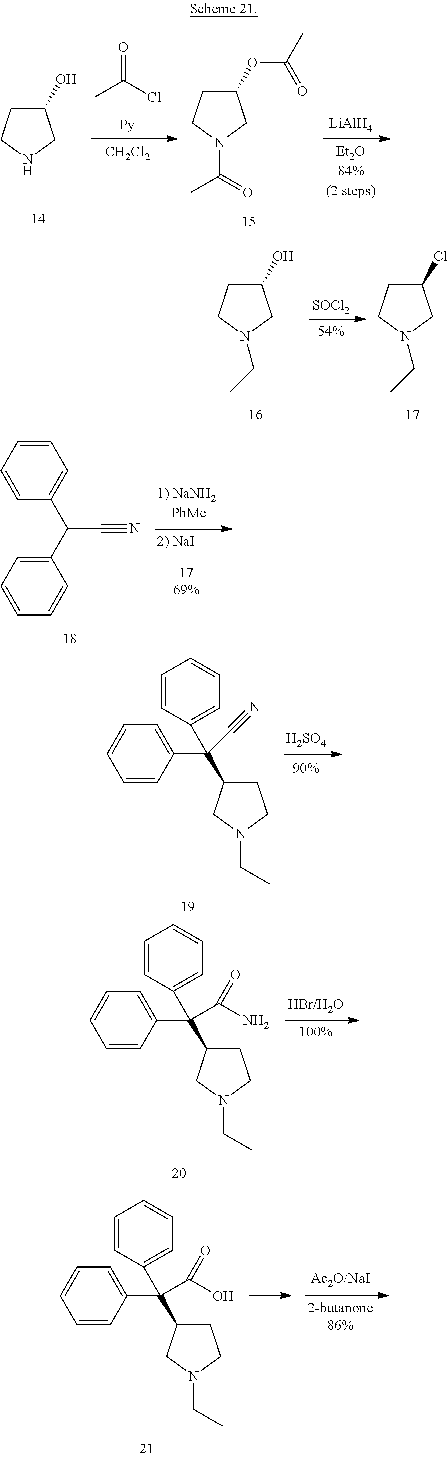

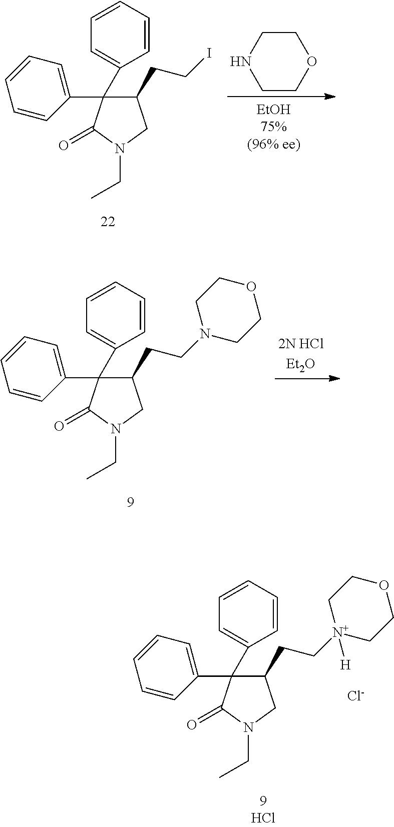

### (S)-1-Ethylpyrrolidin-3-ol (16)

To a solution of (S)-3-hydroxypyrrolidine hydrochloride (14) (5.0 g, 40.46 mmol) and pyridine (18.6 mL, 230.62 mmol) in CH₂Cl₂ (250 mL), acetyl chloride (6.06 mL, 84.97 mmol) CH₂Cl₂ (15 mL) was added in a dropwise manner at 0° C. under an argon atmosphere. After stirring at room temperature for 4 h, the reaction mixture was washed with water (50 mL) and then with saturated NaHCO₃ solution (2×50 mL). The aqueous extracts were saturated with NaCl and then extracted with CH₂Cl₂ (3×100 mL). The combined organic extracts (CH₂Cl₂) were dried (Na₂SO₄) and evaporated. The obtained product (S)-3-acetoxy-1-acetylpyrrolidine (15) was dissolved in diethyl ether (100 mL) and added to a stirred suspension of LiAlH₄ (6.27 g; 165.3 mmol) in Et₂O (120 mL) at 0° C. After stirring for 5 h at room temperature, the reaction mixture was cooled to 0° C. and quenched by the successive additions oh H₂O (2.6 mL), NaOH solution (4 M, 2.0 mL), and H₂O (7.8 mL). After stirring vigorously at room temperature for 4 h, the resulting white precipitate was removed by filtration, and washed on the filter with Et₂O (2×50 mL) and then with CH₂O₂ (2×50 mL). The combined filtrates were collected, dried (anhydrous Na₂SO₄), filtered and evaporated to yield product (S)-1-ethylpyrrolidin-3-ol (16) (3.6 g, 77%), which was used in the next step without purification. 400 MHz ¹H NMR (CDCl₃, ppm) 4.28 (1H, ddd, J=9.8, 5.2, 2.5 Hz) 2.93 (1H, hr s) 2.84-2.78 (1H, m) 2.61 (1H, dd, 2.2 Hz) 2.48-2.45 m) 2.43 (2H, q, J=7.2 Hz) 2.25-2.09 (2H, m) 1.71-1.59 (1H, m) 1.05 (3H, t, J=7.2 Hz).

### (R)-3-Chloro-1-ethylpyrrolidine (17)

(S)-1-Ethylpyrrolidin-3-ol (16) (10.00 g, 86.82 mmol) was added with cooling to SOCl₂ (15 mL). The reaction mixture was heated at reflux for 1 h. After this time, a K₂CO₃ solution (50%, ˜150 mL) was added with cooling and the resulting suspension was extracted with diethyl ether (3×100 mL). The combined organic extracts were washed with water (200 mL), dried over anhydrous Na₂SO₄ and concentrated. The residue was distilled in vacuo (b.p. 54-55° C., 18-20 mmHg) to yield (R)-3-chloro-1-ethylpyrrolidine (17) (6.30 g, 54%). 400 MHz ¹H NMR (CDCl₃, ppm) 4.41-4.34 (1H, m) 3.05 (1H, dd, J=10.7, 6.4 Hz) 2.79-2.69 (2H, m) 2.64-2.46 (3H, m) 2.46-2.36 (1H, m) 2.11-2.02 (1H, m) 1.10 (3H, t, J=7.3 Hz). ESI-MS (m/z): 134, 136 [M+H]⁺.

### (R)-2-(1-Ethylpyrrolidin-3-yl)-2,2-diphenylacetonitrile (19)

A solution of diphenylacetonitrile (18) (872 mg, 4.51 mmol) and sodium amide (229 mg, 5.86 mmol) in dry toluene (7 mL) was heated at reflux for 2 h. The reaction mixture was cooled to the room temperature, and then sodium iodide (743 mg, 4.96 mmol) and (R)-3-chloro-1-ethyl-pyrrolidine (3) (663 mg, 4.96 mmol) were added. The reaction mixture was heated at 110° C. for 20 h. After this time, water (15 mL) was added and the resulting suspension was extracted with EtOAc (3×15 mL). The combined organic extracts were washed with water (50 rut), then with a brine solution (50 mL) and dried over anhydrous Na₂SO₄. The volatiles were removed in vacuo and the residue was purified by flash column chromatography [gradient elution from CH₂Cl₂/MeOH:99:1 (vv) to CH₂Cl₂/MeOH: 4:1 (v/v)] to yield (R)-2-(1-ethylpyrrolidin-3-yl)-2,2-diphenylacetonitrile (19) (900 mg, 69%). 400 MHz ¹H-NMR (CDCl₃, ppm) 7.46-7.40 (4H, m) 7.35-7.27 (4H, m) 7.27-7.21 (2H, m) 3.49-3.40 (1H, m) 3.00 (1H, ddd, J=8.6, 8.6, 3.1 Hz) 2.80 (1H, dd, J=9.5, 7.4 Hz) 2.56-2.35 (3H, m) 2.25 (1H, dd, J=9.5, 9.5 Hz) 2.16-2.04 (1H, m) 1.86-1.77 (1H, m) 1.04 (3H, t, J=7.2 Hz). ESI-MS (m/z): 291 [M+H]⁺.

### (R)-2-(1-Ethylpyrrolidin-3-yl)-2,2-diphenylacetamide (20)

A solution of (R)-(1-ethyl-pyrrolidin-3-yl)-diphenyl-acetonitrile (19) (893 mg, 3.07 mmol) in 70% H₂SO₄ (12 mL) was heated at 120° C. for 4 h. The reaction mixture was poured on ice, made basic with 50% NaOH, and then extracted with CH₂Cl₂ (3×15 mL). The combined organic extracts were washed with water (20 mL) and dried over anhydrous Na₂SO₄. The volatiles were removed in vacuo to yield (R)-2-(1-ethylpyrrolidin-3-yl)-2,2-diphenylacetamide (20) (850 mg, 90%). 400 MHz ¹H NMR (CDCl₃, ppm) 7.62 (1H, hr s) 7.42-7.38 (2H, m) 7.32-7.16 (8H, m) 5.50 (1H, s) 3.48-3.39 (1H, m) 2.77 (1H, d, J=9.7, 5.9 Hz) 2.71-2.65 (1H, m) 2.59-2.51 (1H, m) 2.50-2.32 (3H, m) 1.96-1.89 (2H, m) 1.02 (3H, t, J=7.3 Hz). ESI-MS (m/z): 309 [M+H]⁺.

### (R)-2-(1-Ethylpyrrolidin-3-yl)-2,2-diphenylacetic acid (21)

A solution of (R)-2-(1-ethyl-pyrrolidin-3-yl)-2,2-diphenylacetamide (20) (850 mg, 2.76 mmol) in HBr (30 mL, 48% aqueous solution) was heated at 120° C. for 48 h. After cooling, the solution was made basic with 50% NaOH solution, then acidified to pH 3 by KHSO₄. The resulting mixture was extracted with CH₂Cl₂ (3×15 mL). The combined organic extracts were dried over anhydrous Na₂SO₄ and concentrated. To the resultant residue, hexanes (20 mL) were slowly added to produce a solid which was filtered to yield (R)-(1-ethyl-pyrrolidin-3-yl)-diphenylacetic acid (21) in quantitative yield, which was used in the next step without purification. ESI-MS (m/z): 310 [M+H]⁺.

### (R)-1-Ethyl-4-(2-iodoethyl)-3,3-diphenylpyrrolidin-2-one (21)

A mixture of (R)-(1-ethyl-pyrrolidin-3-yl)-diphenylacetic acid (21) (2.76 mmol), NaI (827 mg, 5.52 mmol) and acetic anhydride (2.0 mL) in 2-butanone (10 mL) was heated at reflux for 2 h. The reaction mixture was cooled and filtered. The filtrate was evaporated and the resultant residue was crystallized from EtOH to yield 1.00 g (86%) of 1-ethyl-4-(2-iodo-ethyl)-3,3-diphenylpyrrolidin-2-one (22). 400 MHz ¹H NMR (CDCl₃, ppm) 7.57-7.53 (2H, m) 7.37-7.31 (2H, m) 7.30-7.17 (4H, m) 6.88-6.84 (2H, m) 3.64-3.54 (1H, m) 3.53 (1H, dd, J=8.9, 7.3 Hz) 3.47-3.38 (2H, m) 3.23 (1H, ddd, J=10.0, 6.4, 4.4 Hz) 3.04-2.94 (2H, m) 2.02-1.92 (1H, m) 1.23 (3H, t, J=7.3 Hz) 1.19-1.09 (1H, m). ESI-MS (m/z): 420 [M+H]⁺.

### (R)-1-Ethyl-4-(2-morpholinoethyl)-3,3-diphenylpyrrolidin-2-one (9)

A mixture of (R)-1-ethyl-4-(2-iodoethyl)-3,3-diphenylpyrrolidin-2-one (22) (200 mg, 0.48 mmol) and morpholine (125 μL, 1.44 mmol) in EtOH (2 mL) was heated in a closed vial at 105° C. for 4 h. After this time, the volatiles were removed in vacuo and water (10 mL) was added. The resulting suspension was extracted with EtOAc (3×10 mL). The combined organic extracts were washed with water (2×20 mL), then with a brine solution and dried over anhydrous Na₂SO₄. The crude product was purified by flash column chromatography [gradient elution from EtOAc to EtOAc/MeOH:4:1 (v/v)] to yield (R)-1-ethyl-4-(2-morpholinoethyl)-3,3-diphenylpyrrolidin-2-one (9) (137 mg, 75%). 400 MHz ¹H NMR (CDCl₃, ppm) 7.55-7.51 (2H, m) 7.36-7.30 (2H, m) 7.29-7.17 (4H, m) 6.92-6.87 (2H, m) 3.72-3.68 (4H, m) 3.64-3.51 (2H, m) 3.48-3.38 (1H, m) 3.35-3.26 (1H, m) 3.04 (1H, dd, J=9.0, 9.0 Hz) 2.44-2.26 (6H, m) 1.71-1.62 (1H, m) 1.23 (3H, t, J=7.2 Hz) 0.92-0.80 (1H, ESI-MS (m/z): 379 [M+H]⁺.

### Example 10

### Chiral Synthesis of (R)-Doxapram from (R)-4-Ethyl-3-pyrrolidinol and Chiral Analytical HPLC Methods

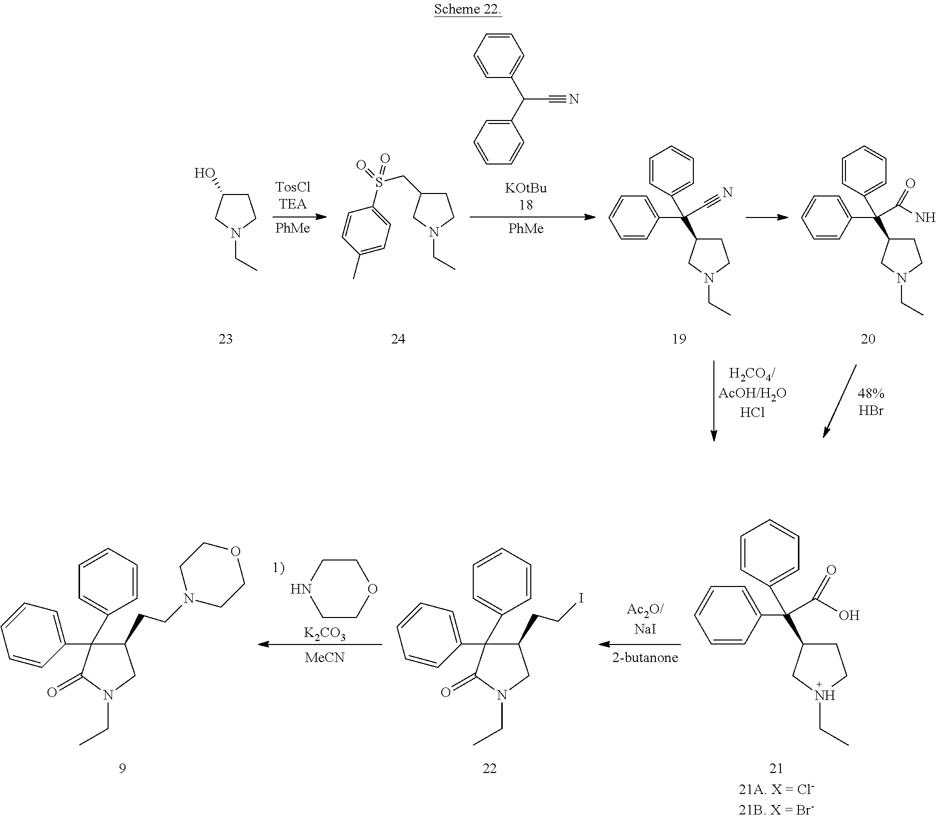

### (R)-1-ethylpyrrolidin-3-yl 4-methylbenzenesulfonate (24)

p-Toluenesulfonyl chloride (3.43 g, 17.63 mmol) in toluene (15 mL) was added to a solution of (R)-1ethyl-3-pyrrolidinol (23) (1.93 g, 16.79 mmol) and triethylamine (4.7 mL, 33.59 mmol) in toluene (15 mL) under argon atmosphere at 0° C. The reaction mixture was stirred at ambient temperature for 16 h. The precipitated triethylammonium chloride was filtered off. Saturated NaHCO₃ solution (25 mL) was added to the filtrate, and the resulting mixture was stirred at room temperature for 1 hour. The mixture was extracted with EtOAc (3×50 mL). The combined organic extracts were washed with saturated NH₄Cl solution (75 mL), water (75 mL) and brine (50 mL). The organic phase was dried over MgSO₄, the solvent was removed in vacuo to yield 4.15 g (91%) of (R)-1-ethylpyrrolidin-3-yl 4-methylbenzenesulfonate (24) with >99% ee (Analytical Method A). HPLC purity: 94%. 400 MHz ¹H NMR (CDCl₃, ppm) 7.79-7.75 (2H, m), 7.36-7.32 (2H, m), 5.04-4.98 (1H, m), 3.22-3.13 (1H, m), 192-2.78 (3H, m), 2.77-2.63 (2H, m), 2.44 (3H, s), 2.29-2.17 (1H, m), 2.06-1.97 (1H, m), 1.17 (3H, t, J=7.2 Hz). ESI-MS (m/z): 270 [M+H]⁺.

### (R)-1-Ethylpyrrolidin-3-yl 4-methylbenzenesulfonate (24)

p-Toluenesulfonyl chloride (348.0 g, 1.83 mol) in THF (1000 mL) was added portion-wise to the stirred solution of (R)-1-ethyl-3-pyrrolidinol (23) (200.0 g, 1.74 mol) and triethylamine (364 mL, 2.61 mol) in THF (800 mL) under argon atmosphere at 0° C. The reaction mixture was stirred at ambient temperature for 16 h. The precipitated triethylammonium chloride was filtered off; the filter cake was washed with THF (200 mL), Saturated NaIHCO₃ solution (800 mL) was added to the filtrate, and the resulting mixture was stirred at room temperature for 2 h. The mixture was evaporated to about half of the original volume and extracted with EtOAc (3×500 mL). The combined organic extracts were washed with saturated NH₄Cl solution (800 mL), water (800 mL) and brine (500 mL). The organic phase was dried over MgSO₄, and the solvent was removed in vacuo to yield (R)-1-ethyl pyrrolidin-3-yl-4-methylbenzenesulfonate (24) with 92% ee (Analytical Method A). (412.0 g, 88%) which was used in the next stage without purification. 400 MHz ¹H NMR (CDCl₃, ppm) 7.79-7.75 (2H, m), 7.36-7.32 (2H, m), 5.04-4.98 (1H, m), 3.22-3.13 (1H, m), 2.92-2.78 (3H, m), 2.77-2.63 (2H, m), 2.44 (3H, s), 2.29-2.17 (1H, m), 2.06-1.97 (1H, m), 1.17 (311, t, J=7.2 Hz). ESI-MS (m/z): 270 [M+H]⁺.

### (R)-2-(1-Ethylpyrrolidin-3-yl)-2,2-diphenylacetonitrile (19)

A solution of diphenylacetonitrile (18) (3.09 g; 16.00 mmol) and KOtBu (2.57 g, 22.86 mmol) in toluene (30 mL) was stirred at 0° C. for 30 min. (R)-1-Ethylpyrrolidin-3-yl-4-methylbenzenesulfonate (24) (4.10 g, 15.24 mmol) in toluene (30 mL) was added the reaction mixture was heated at 90° C. for 3 h, then cooled to the ambient temperature. The mixture was extracted with 2N H₂SO₄ solution (3×30 mL). The combined aqueous extracts were washed with EtOAc (50 mL), then made basic (pH ˜10) by adding 4M NaOH solution. The resulting suspension was extracted with EtOAc (3×30 mL). Combined organic extracts were washed with water (50 mL), brine (50 mL), and dried over MgSO₄. The volatiles were removed in vacuo to yield (R)-2-(1-ethylpyrrolidin-3-yl)-2,2-diphenylacetonitrile (19) with >99% ee (Analytical Method A) (3.41 g, 77%). HPLC purity: 98%. 400 MHz ¹H NMR (CDCl₃, ppm) 7.46-7.40 (4H, m) 7.35-7.27 (4H, m) 7.27-7.21 (2H, m) 3.49-3.40 (1H, m) 3.00 (1H, ddd, J=8.6, 8.6, 3.2 Hz) 2.80 (1H, dd, J=9.5, 7.4 Hz) 2.56-2.35 (3H, m) 2.25 (1H, dd, J=9.5, 9.5 Hz) 2.16-2.04 (1H, m) 1.86-1.77 (1H, m) 1.04 (3H, t, J=7.2 Hz). ESI-MS (m/z): 291 [M+H]+.

### (R)-2-(1-Ethylpyrrolidin-3-yl)-2,2-diphenylacetonitrile (19)

A solution of diphenylacetonitrile (18) (151.0 g, 0.78 mol) and KOtBu (124.0 g, 1.11 mol) in toluene (1000 mL) was stirred at 0° C. for 1 h. (R)-1-Ethylpyrrolidin-3-yl 4-methylbenzenesulfonate (24) (200 g, 0.74 mot) in toluene (1000 mL) was added portion-wise over 40 min. The reaction mixture was heated at 90° C. for 4 h, then cooled to the ambient temperature, and water (600 mL) was added. The organic layer was separated and extracted with 2N H₂SO₄ solution (3×700 mL). The combined aqueous extracts were made basic (pH ˜1.0) using 4M NaOH solution and extracted with EtOAc (3×600 mL). Combined organic extracts were washed with water (1000 mL), brine (600 mL), and dried over MgSO₄. The volatiles were removed in vacuo to yield (R)-2-(1-ethylpyrrolidin-3-yl)-2,2-diphenylacetonitrile (19) with 92% ee (Analytical Method A) (178.0 g, 83%), which was used in the next stage without purification, 400 MHz ¹H-NMR (CDCl₃, ppm) 7.46-7.40 (4H, m) 7.35-7.27 (4H, m) 7.27-7.21 (2H, m) 3.49-3.40 (1H, m) 3.00 (111, ddd, 8.6, 3.2 Hz) 2.80 (1H, dd, J=9.5, 7.4 Hz) 2.56-2.35 (3H, m) 2.25 (1H, dd, J=9.5, 9.5 Hz) 2.16-2.04 (1H, m) 1.86-1.77 (1H, m) 1.04 (3H, t, J=7.2 Hz). 100 MHz ¹³C-NMR (CDCl₃, ppm) 140.5, 140.4, 129.5, 128.2, 127.0, 126.6, 121.9, 57.4, 57.3, 53.6, 49.6, 43.5, 28.8, 14.1. ESI-MS (m/z): 291 [M+H].

### (R)-2-(1-Ethylpyrrolidin-3-yl)-2,2-diphenylacetonitrile (19) (Telescoped Procedure)

Triethylamine (18.4 mL, 132.20 mmol) was added to a solution of (R)-1-ethyl-3-pyrrolidinol (23) (10.15 g, 88.13 mmol) in toluene (125 mL). p-Toluenesulfonyl chloride (18.00 g, 92.54 mmol) was then added portion-wise to the reaction mixture under argon atmosphere at 0° C. The reaction mixture was stirred at ambient temperature for 16 h. The precipitated triethylammonium chloride was filtered off; the filter cake was washed with toluene (35 mL). The combined toluene solutions containing (R)-1-ethylpyrrolidin-3-yl-4-methylbenzenesulfonate (24) was used in the next step without isolation. The analytical sample was isolated (99% ee as per Analytical method A). 400 MHz ¹H NMR (CDCl₃, ppm) 7.79-7.75 (2H, m), 7.36-7.32 (2H, m), 5.04-4.98 (1H, m), 3.22-3.13 (1H, 2.92-2.78 (3H, m), 2.77-2.63 (2H, m), 2.44 (3H, s), 2.29-2.17 (1H, m), 2.06-1.97 (1H, m), 1.17 (3H, t, J=7.2 Hz). ESI-MS (m/z): 270 [M+H]⁺.

A solution of diphenylacetonitrile (18) (17.88 g; 92.54 mmol) and KOtBu (15.29 g, 132.2 mmol) in toluene (100 mL) was stirred at 0° C. for 30 min. The toluene solution containing (R)-1-ethylpyrrolidin-3-yl-4-methylbenzenesulfonate (24) was added portion wise over 20 min. The resulting reaction mixture was heated at 90° C. for 4 hours, then cooled to the ambient temperature and water (150 mL) was added. The organic layer was separated and extracted with 2N H₂SO₄ solution (3×100 mL). The combined aqueous extracts were made basic (pH ˜10) using 4M NaOH solution and extracted with EtOAc (3×150 mL). The combined organic extracts were washed with water (200 mL), brine (200 and dried over MgSO₄. The volatiles were removed in vacuo to yield compound (19) with >99% ee (as per Analytical Method A (20.80 g, 81% in 2 steps starting from (R)-1-ethyl-3-pyrrolidinol (1)). 400 MHz ¹H NMR (CDCl₃, ppm) 7.46-7.40 (4H, m) 7.35-7.27 (4H, m) 7.27-7.21 (2H, m) 3.49-3.40 (1H, m) 3.00 (1H, ddd, J=8.6, 8.6, 3.2 Hz) 2.80 (1H, dd, J=9.5, 7.4 Hz) 2.56-2.35 (1H, m) 2.25 (1H, dd, J=9.5, 9.5 Hz) 2.16-2.04 (1H, m) 1.86-1.77 (1H, m) 1.04 (3H, t, J=7.2 Hz). 100 MHz ¹³C NMR (CDCl₃, ppm) 140.5, 140.4, 129.5, 128.2, 127.0, 126.6, 121.9, 57.4, 57.3, 53.6, 49.6, 43.5, 28.8, 14.1. ESI-MS (m/z): 291 [M+H]⁺.

### Chiral Enrichment of (R)-2-(1-Ethylpyrrolidin-3-yl)-2,2-diphenylacetonitrile (19)

(R)-2-(1-ethylpyrrolidin-3-yl)-2,2-diphenylacetonitrile (92% ee, 1.00 g, 3.44 mmol) was dissolved in acetone (12 mL), D-DBTA×H₂O (907 mg, 2.41 mmol, 0.7 eq) in acetone (5 mL) was added dropwise. The mixture was stirred for 30 minutes. The solvent was removed in vacuo till Y of volume; the mixture was stirred for another 30 minutes with cooling (0° C.). The precipitate was filtered, washed with cold acetone (10 mL) and dried to afford (R)-2-(1-ethylpyrrolidin-3-yl)-2,2-diphenylacetonitrile. D-DBTA salt (830 mg salt, 515 mg of (19) free base, 1.77 mmole, 51%), cc of 96.3% (Analytical Method A). NMR supports a salt comprising two moles of 19 per moles of DBTA.

The product was re-crystallized EtOAc/EtOH (9/1), ee of 98.2 (Analytical Method A). 400 MHz ¹H NMR (CDCl₃, ppm) 8.21-8.17 (2H, m) 7.50-7.45 (1H, m) 7.45-7.39 (4H, m) 7.38-7.33 (2H, m) 7.33-7.27 (4H, m) 7.27-7.20 (2H, m) 6.03 (1H, s) 3.90-3.78 (1H, m) 3.60-3.48 (2H, m) 2.93-2.79 (2H, m) 2.78-2.69 (1H, m) 2.49 t, J=10.4 Hz) 2.28-2.15 (1H, m) 1.88-1.78 (1H, s) 1.09 (31-1, t, J=7.2 Hz). Melting point: 157° C. (decomposed). ESI-MS (m/z): 291 [M+H]⁺; 357 [M−H]⁻.

(R)-2-(1-Ethylpyrrolidin-3-yl)-2,2-diphenylacetic acid hydrochloride (21A)

(R)-2-(1-Ethylpyrrolidin-3-yl)-2,2-diphenylacetonitrile (19) (20 g, 68.87 mmol) was suspended in the mixture of H₂SO₄ (100 mL), AcOH (50 mL) and water (50 mL), and heated at 140° C. for 72 h. The cooled solution was poured on ice (200 mL), made basic (pH ˜10) with 25% NaOH solution, then washed with CH₂Cl_{2×150} nit). The aqueous phase was acidified to pH 3 by cone. HCl. The resulting mixture was extracted with CH₂Cl₂ (3×200 mL). The combined organic extracts were dried over MgSO₄ and concentrated. The residue was treated with hexanes, filtrated to yield (R)-(1-ethyl-pyrrolidin-3-yl)-diphenylacetic acid hydrochloride (21A) (16.70 g, 70%), which 1 was used in the next step without purification. ESI-MS (m/z): 310 [M+H]⁺.

### (R)-2-(1-Ethylpyrrolidin-3-yl)-2,2-diphenylacetic acid hydrobromide (21B)

A solution of (R)-(1-ethyl-pyrrolidin-3-yl)-diphenylacetonitrile (19) (136.8 g, 0.47 mol) in 80% H₂SO₄ (600 mL) was heated at 120° C. for 8 h. The reaction mixture was poured on ice (600 mL), and made basic (pH 10) with 25% NaOH (˜1200 mL). The precipitated Na₂SO₄ was filtered off, and the filtrate was extracted with CH₂Cl₂ (3×600 mL). Combined organic extracts were washed with water (2×600 mL), and dried over MgSO₄. The volatiles were removed in vacuo to yield (R)-2-(1-ethylpyrrolidin-3-yl)-2,2-diphenylacetamide (20) (137.1 g, 95%), which was used in the next stage without purification. 400 MHz ¹H NMR (CDCl₃, ppm) 7.60-7.45 (111, br s) 7.42-7.38 (2H, m) 7.32-7.16 (8H, m) 5.78 (1H, s) 3.49-3.39 (1H, m) 2.77 (1H, d, J=9.8, 6.0 Hz) 2.70 (111, t, J=9.5 Hz) 2.59-2.50 (1H, m) 2.50-2.32 (3H, m) 1.96-1.89 (2H, m) 1.01 (3H, t, J=7.2 Hz). ESI-MS (m/z): 309 [M+H]⁺.

A solution of (R)-2-(1-ethyl-pyrrolidin-3-yl)-2,2-diphenylacetamide (20) (136 g, 0.44 mol) in 48% HBr (600 mL, water solution) was heated at 120° C. for 70 h. The reaction mixture was cooled to the room temperature, poured on ice (500 ml) and the pH of the solution was adjusted to pH 3 with 25% NaOH solution. The resulting mixture was extracted with CH₂Cl₂ (3×500 mL). The combined organic extracts were dried over MgSO₄ and concentrated to afford (R)-(1-ethyl-pyrrolidin-3-yl)-diphenyl-acetic acid as hydrobromide salt (21B) (139.5 g, 81%) ESI-MS (m/z): 310 [M+H]⁺.

### (R)-1-Ethyl-4-(2-iodo-ethyl)-3,3-diphenyl-pyrrolidin-2-one (22)

A mixture of (1-ethyl-pyrrolidin-3-yl)-diphenylacetic acid hydrobromide (21B) (27.0 g, 69.18 mmol), NaI (20.8 g, 138.77 mmol) and acetic anhydride (40 mL) in 2-butanone (185 mL) was heated at reflux for 2 h. The reaction mixture was cooled to the room temperature and filtered. The filtrate was evaporated, the residue was suspended in saturated NaHCO₃ solution (200 mL) and extracted with EtOAc (3×150 mL). The combined organic extracts were washed with water (2×150 mL) and brine (200 mL), dried over Na₂SO₄, and concentrated to yield 1-ethyl-4-(2-iodo-ethyl)-3,3-diphenyl-pyrrolidin-2-one (22) (25.2 g, 87%). 400 MHz ¹H NMR (CDCl₃, ppm) 7.57-7.53 (2H, m) 7.37-7.31 (2H, m) 7.30-7.17 (4H, m) 6.88-6.84 (2H, m) 3.64-3.54 (1H, m) 3.53 (1H, dd, J=8.9, 7.3 Hz) 3.47-3.38 (2H, m) 3.23 (1H, ddd, J=10.0, 6.4, 4.4 Hz) 3.04-2.94 (2H, m) 2.02-1.92 (1H, m) 1.23 (3H, t, J=7.3 Hz) 1.19-1.09 (1H, m). ESI-MS (m/z): 420 [M+H]⁺.

### (R)-1-Ethyl-4-(2-morpholinoethyl)-3,3-diphenylpyrrolidin-2-one (9)

The reaction mixture of (R)-1-ethyl-4-(2-iodoethyl)-3,3-diphenyl pyrrolidin-2-one (22) (25.2 g, 60.01 mmol), K₂CO₃ (24.9 g, 180.30 mmol) and morpholine (6.3 mL, 72.12 mmol) in acetonitrile (250 mL) was heated at reflux for 3 h. Volatiles were removed under vacuum and water (150 mL) was added. The resulting suspension was extracted with EtOAc (3×150 mL). The combined organic extracts were washed with water (2×150 mL), brine (150 mL), and dried over Na₂SO₄. The product was purified by flash column chromatography using gradient elution from EtOAc/MeOH (100/0) to EtOAc/MeOH (80/20) to yield (R)-1-ethyl-4-(2-morpholino ethyl)-3,3-diphenylpyrrolidin-2-one (9) (20.1 g (88%) 400 MHz ¹H NMR (CDCl₃, ppm) 7.55-7.51 (2H, m) 7.36-7.30 (2H, m) 7.29-7.17 (4H, m) 6.92-6.87 (2H, m) 3.72-3.68 (4H, m) 3.64-3.51 (2H, m) 3.48-3.38 (1H, m) 3.35-3.26 (1H, m) 3.04 (1H, dd, J=.0, 9.0 Hz) 2.44-2.26 (6H, m) 11.71-1.62 (1H, m) 1.23 (3H, t, J=7.2 Hz) 0.92-0.80 (1H, m). ESI-MS (m/z): 379 [M-1-14].

### Analytical Method A

### Chiral HPLC Method for (R)-1-Ethylpyrrolidin-3-yl 4-methylbenzenesulfonate (24) and (R)-2-(1-Ethylpyrrolidin-3-yl)-2,2-diphenylacetonitrile (19)

Column: Daicel Chiralpak® IA; Eluent: hexane/iPrOH −98/2+0.1% DEA; Flow: 0.9 mL/min; Sample: 1 mg/ml; Detection: 228 nm. A sample trace for chiral HPLC analysis of (R,S)-2-(1-Ethylpyrrolidin-3-yl)-2,2-diphenylacetonitrile (19) is illustrated in FIG. 16.

### Analytical Method B

### Chiral HPLC Method for (R)-1-Ethyl-4-(2-iodo-ethyl)-3,3-diphenyl-pyrrolidin-2-one (22)

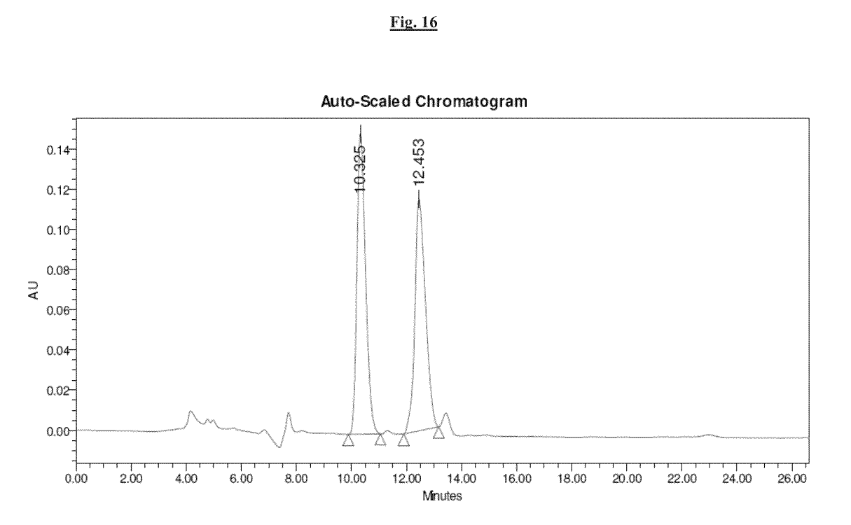

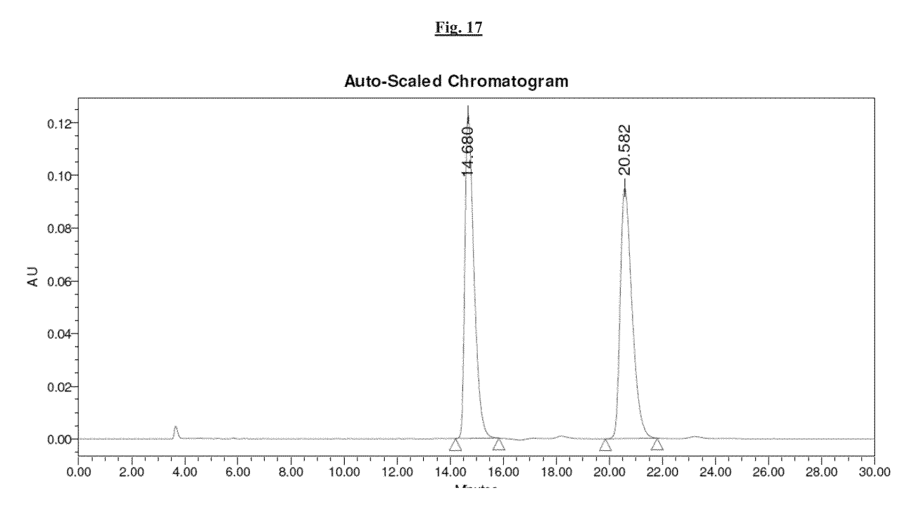

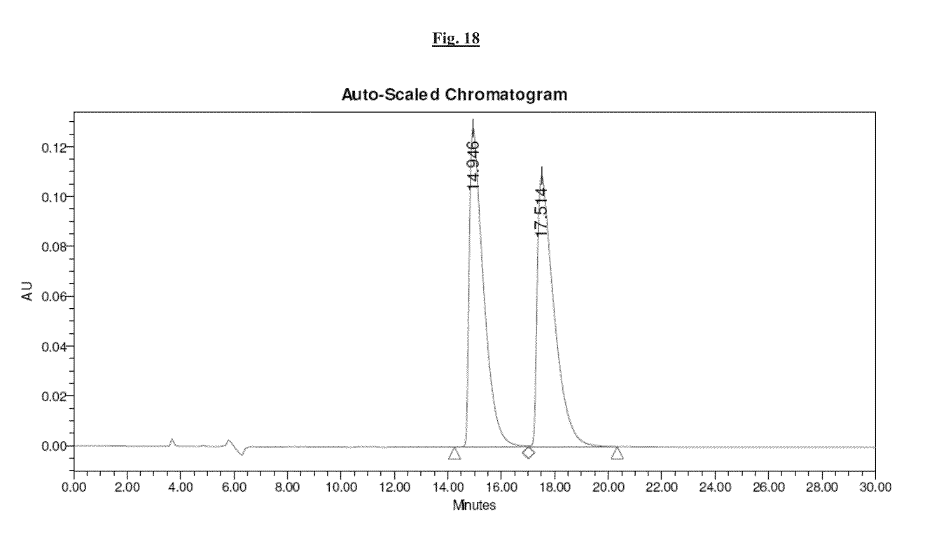

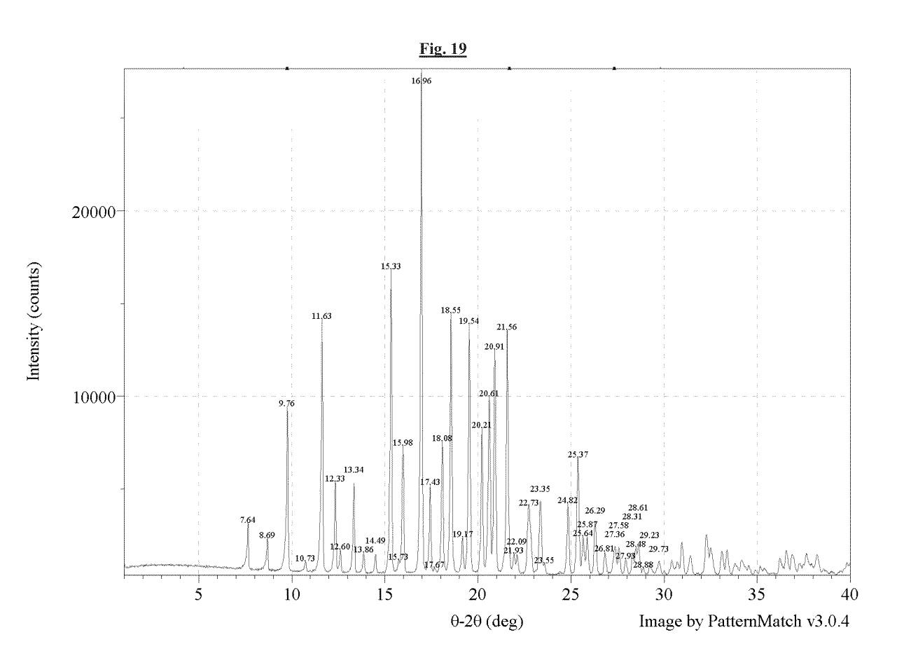

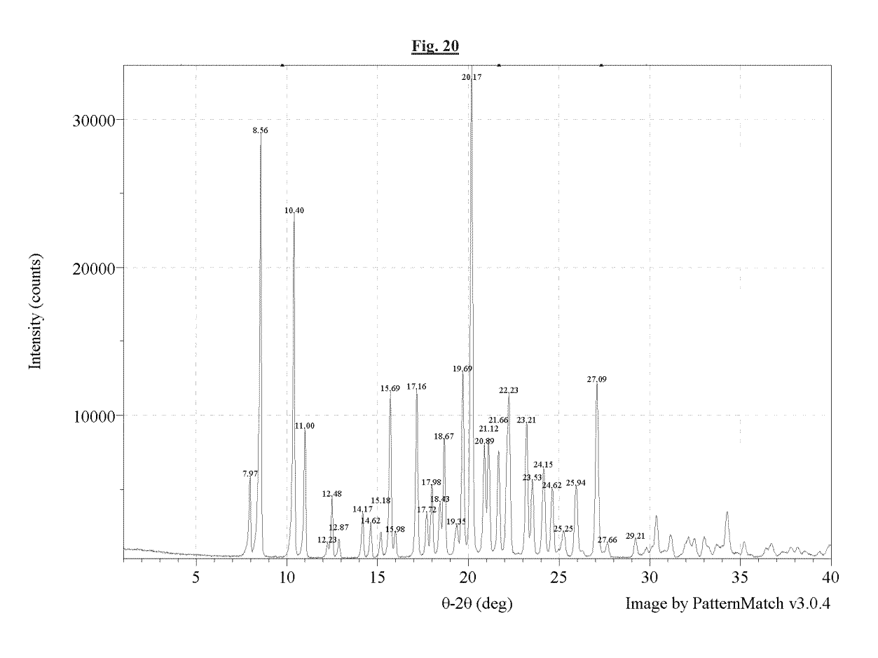

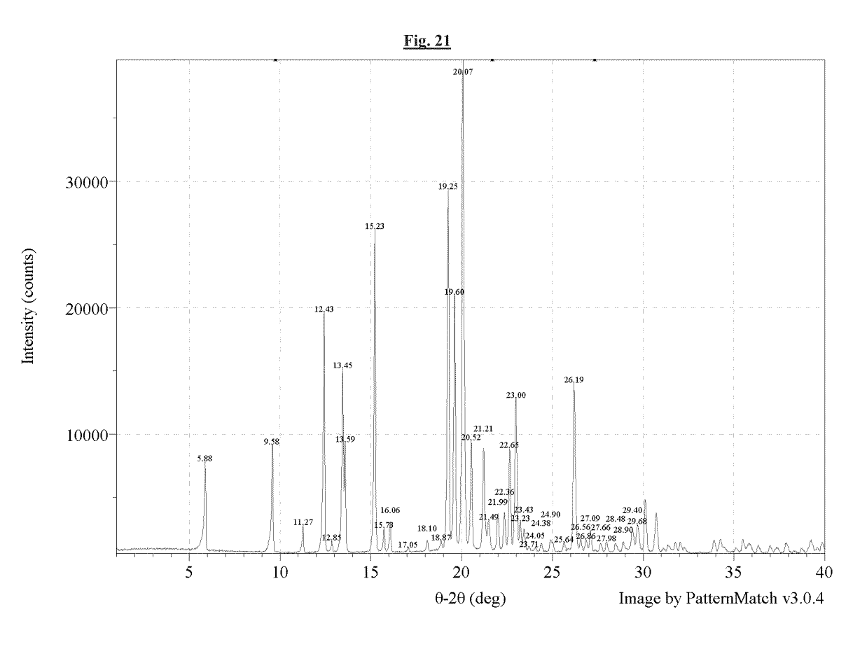

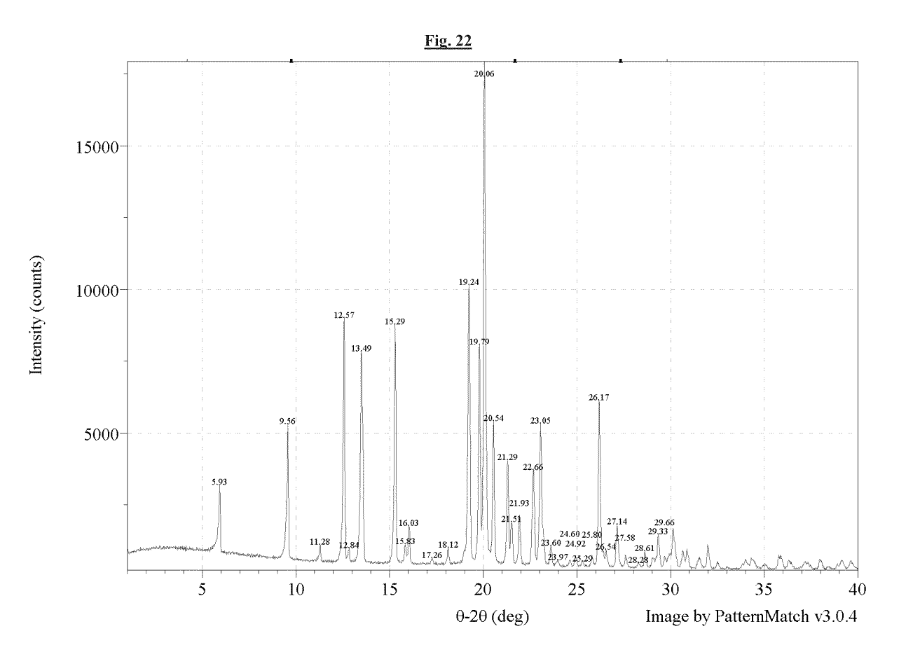

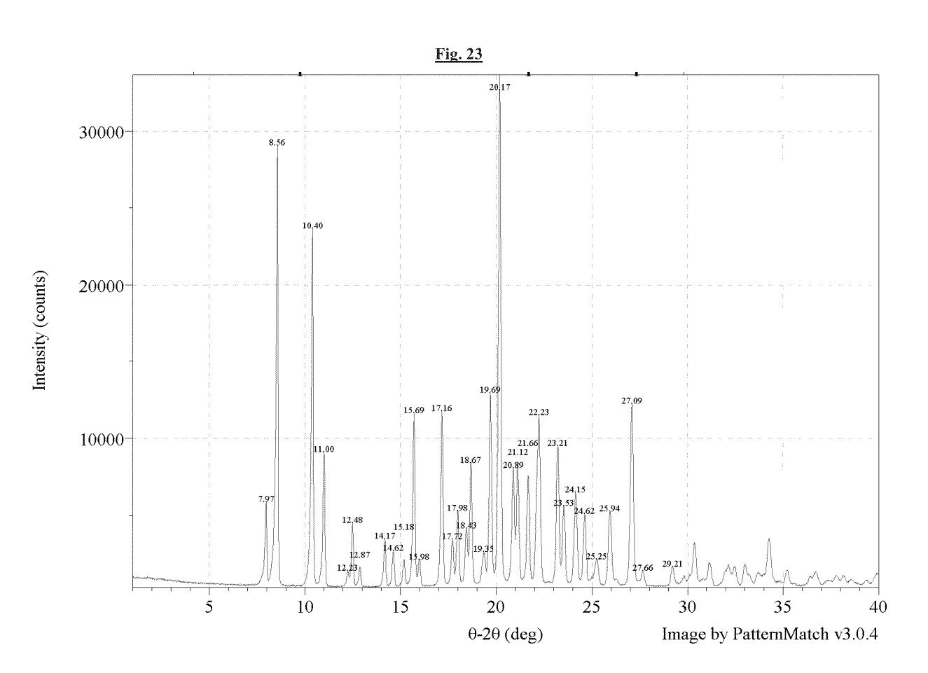

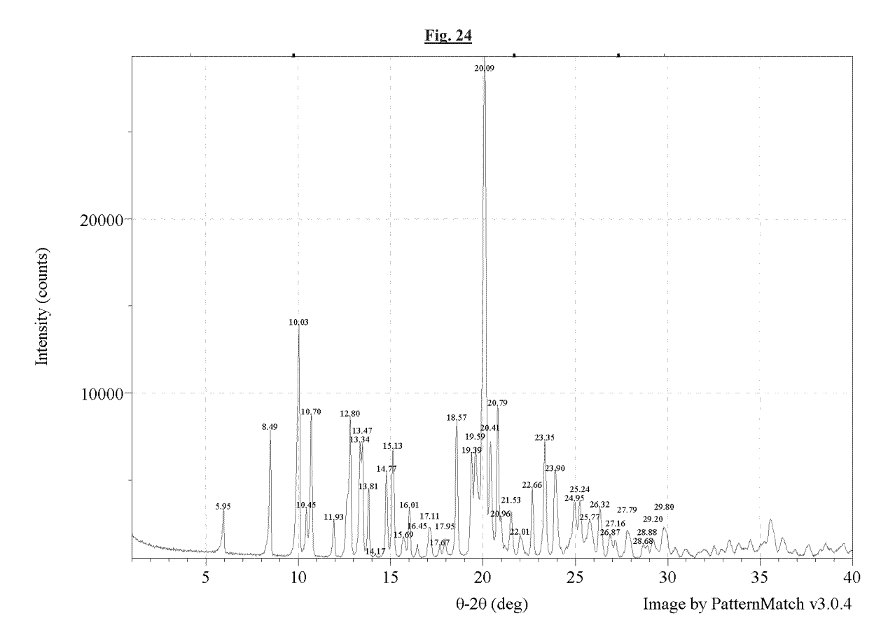

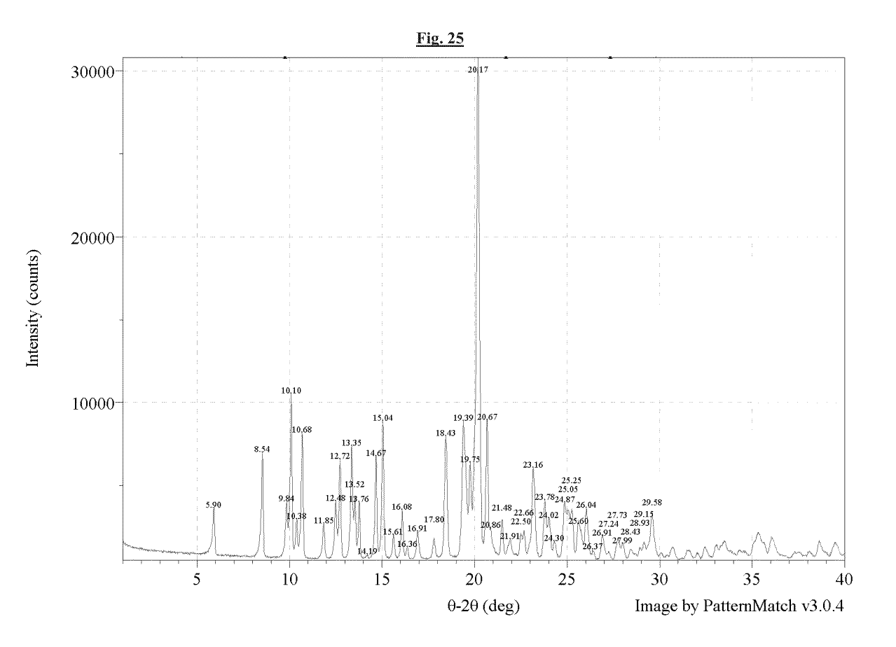

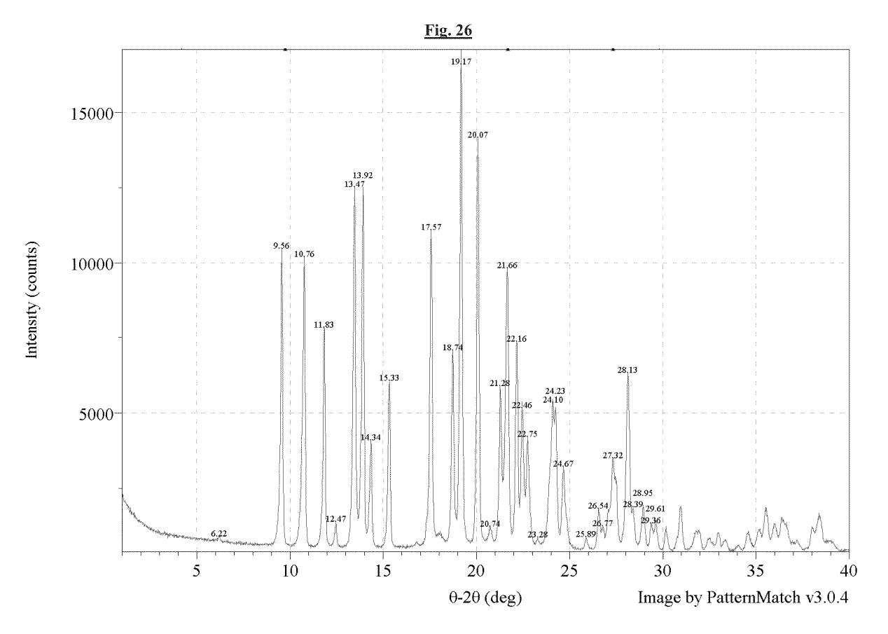

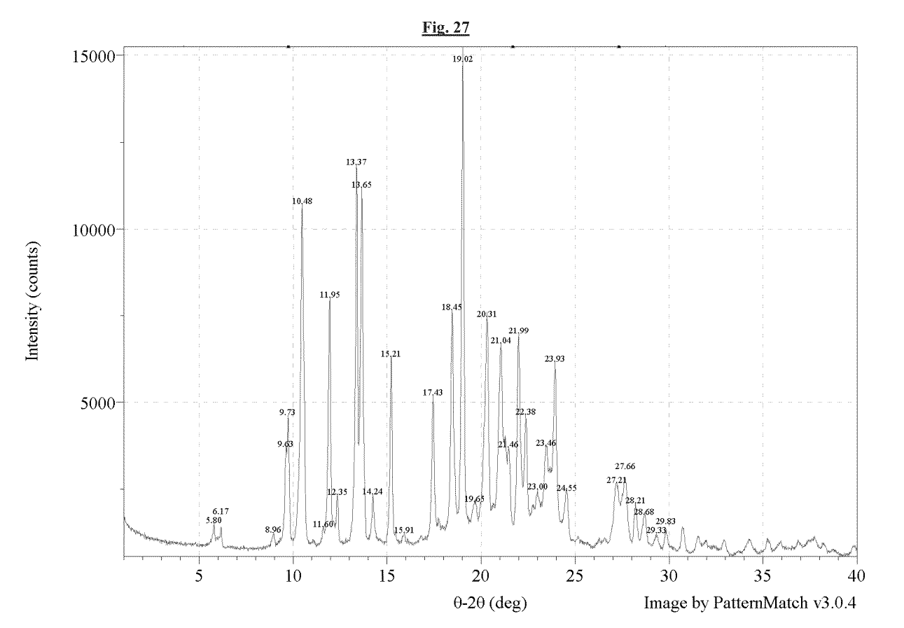

Column: Daicel Chiralpak® IA; Eluent: 70% A/30% B; A) 100% Hex; B) 20% IPA. 80% Hex. A sample trace for chiral HPLC analysis of (R,S)-1-ethyl-4-(2-iodo-ethyl)-3,3-diphenyl-pyrrolidin-2-one (22) is illustrated in FIG. 17.

### Analytical Method C

### Chiral HPLC Method for (+)Doxapram

Column: Daicel Chiralpak® IA; Eluent: 60% A/40% B; A) 100% Hex, 0.1% DEA; B) 20% IPA, 80% Hex. A sample trace for chiral HPLC analysis of (±)-doxapram is illustrated in FIG. 18.

### Example 11

### Synthesis of d₈-(+)-Doxapram

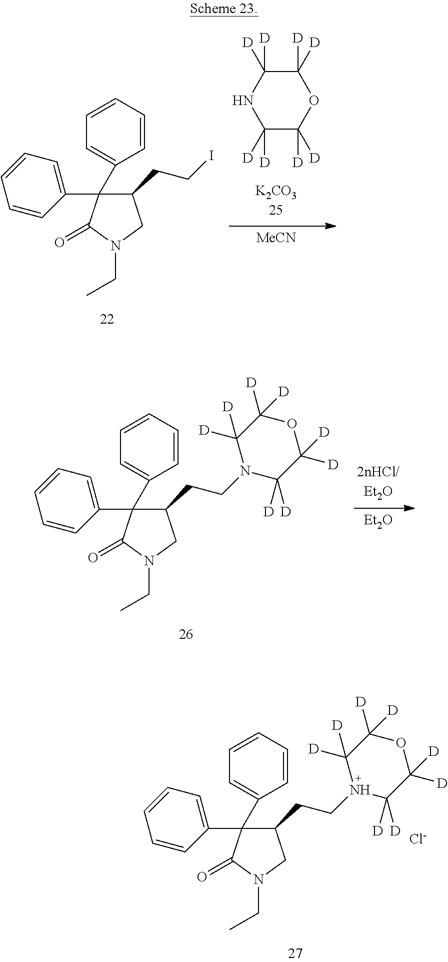

### (R)-1-Ethyl-4-(2-(2,2,3,3,5,5,6,6-octadeuterio-4-morpholino)ethyl)-3,3-diphenylpyrrolidin-2-one (22)

The mixture of (R)-1-ethyl-4-(2-iodoethyl)-3,3-diphenylpyrrolidin-2-one (22) (500 mg, 1.19 mmol), morpholine-d₈ (25) (136 mg, 1.43 mmol) and K₂C01 (412 mg, 2.98 mmol) in acetonitrile (10 mL) was heated in the closed vial at 90° C. for 3 h. The mixture was cooled to room temperature and then water (50 mL) was added. The resulting suspension was extracted with EtOAc (3×20 mL). The combined organic extracts were washed with water (2×20 mL), then with a brine solution (25 mL) and dried over anhydrous Na₂SO₄. The crude product was purified by flash column chromatography (gradient elution from EtOAc to EtOAc/MeOH:95/5 (v/v) to yield (R)-1-ethyl-4-(2-morpholinoethyl)-3,3-diphenylpyrrolidin-2-one-d₈ (26) (350 mg, 76%). ESI-MS (m/z): 387 [M+H]⁺.

### (R)-4-Ethyl-4-(2-(2,2,3,3,5,5,6,6-octadeuterio-4-morpholino)ethyl)-3,3-diphenylpyrrolidin-2-one (27)

(R)-1-Ethyl-4-(2-morpholinoethyl)-3,3-diphenylpyrrolidin-2-one-dₛ (26) (328 mg, 0.85 mmol) was dissolved in Et₂O (5 mL), and 2N HCl/Et₂O (415 μL, 0.83 mmol) was added. The mixture was stirred at room temperature for 30 min. The resultant precipitate was filtered, washed with Et₂O (15 mL), dried at 100° C. for 2 h to yield (R)-1-ethyl-4-(2-morpholin-4-yl-ethyl)-3,3-diphenyl-pyrrolidin-2-one-d₈ hydrochloride (27) (320 mg, 91%). 400 MHz ¹H NMR (CDCl₃, ppm) 13.03 (1H, s) 7.52-7.47 (2H, s) 7.36-7.30 (2H, m) 7.30-7.21 (4H, m) 7.08-7.03 (2H, m) 3.63 (1H, dd, J=9.8, 6.7 Hz) 3.51-3.34 (2H, m) 3.32-3.23 (1H, m) 3.16 (1H, dd, J=9.8, 5.4 Hz) 2.81-2.70 (1H, m) 2.57-2.47 (1H, m) 1.93-1.82 (1H, m) 1.72-1.61 (1H, m) 1.13 (3H, t, J=7.2 Hz), ESI-MS (m/z): 387 [M+H]⁺. HPLC (Daicel Chiralpak® IA; eluent: hexane/iPrOH 98/2 (v/v) with 0.1% DEA; flow rate: 0.9 mL/min; sample concentration: 1 mg/ml; detection wavelength: 228 nm): 99% ee.

### Example 12

### Salt of (+)-Doxapram

### D-glucuronate Salt (28)

(+)-Doxapram free base (9, 308 mg, 0.814 mmole) was dissolved in acetone (2.5 mL) at ambient temperature to yield a clear solution. To this clear solution, a solution of D-glucuronic acid (160 mg, 0.824 mmole) in water (2.5 mL) was added. The resulting clear solution was stirred at ambient temperature for ˜2 days prior to vacuum filtration to collect the solid product. The isolated solids were vacuum dried for ˜1 day (144.5 mg, 31%).

DSC (10° C./min): sharp strong endotherm with two maxima at ˜191.8 and ˜192.2° C., possibly attributed to two melting events or a result of decomposition. Elemental analysis—theoretical (for 1:1 salt): 62.92% C, 7.04% H, 4.89% N. found: 62.90% C, 7.26% FL, 4.82% N. Table 20 summarizes prominent XRPD signals for (+)-doxapram glucuronate.

TABLE 20
Summary of XRPD signals for (+)-doxapram glucuronate

| 2θ | d space (Å) | Intensity (%) |
|---|---|---|
| 7.64 ± 0.20 | 11.574 ± 0.311 | 11 |
| 8.69 ± 0.20 | 10.174 ± 0.239 | 8 |
| 9.76 ± 0.20 | 9.061 ± 0.189 | 34 |
| 11.63 ± 0.20 | 7.607 ± 0.133 | 51 |
| 12.33 ± 0.20 | 7.176 ± 0.118 | 19 |
| 13.34 ± 0.20 | 6.639 ± 0.101 | 19 |
| 15.33 ± 0.20 | 5.782 ± 0.076 | 61 |
| 15.98 ± 0.20 | 5.547 ± 0.070 | 27 |
| 16.96 ± 0.20 | 5.227 ± 0.062 | 100 |
| 17.43 ± 0.20 | 5.088 ± 0.059 | 19 |
| 18.08 ± 0.20 | 4.906 ± 0.054 | 27 |
| 18.55 ± 0.20 | 4.783 ± 0.052 | 52 |
| 19.54 ± 0.20 | 4.544 ± 0.047 | 50 |
| 20.21 ± 0.20 | 4.395 ± 0.043 | 30 |
| 20.61 ± 0.20 | 4.310 ± 0.042 | 36 |
| 20.91 ± 0.20 | 4.249 ± 0.041 | 45 |
| 21.56 ± 0.20 | 4.122 ± 0.038 | 49 |
| 22.73 ± 0.20 | 3.912 ± 0.034 | 15 |
| 23.35 ± 0.20 | 3.810 ± 0.032 | 16 |
| 24.82 ± 0.20 | 3.588 ± 0.029 | 15 |
| 25.37 ± 0.20 | 3.511 ± 0.027 | 24 |

### Sulfate Salt (29)

(+)-Doxapram free base (9, 508 mg, 1.34 mmoles) was dissolved in 2-propanol (15 mL) at ambient temperature to yield a clear solution. To this clear solution, concentrated (˜18 M) sulfuric acid (75 μL, 1.53 mmole) was added. The resulting clear solution was stirred at ambient temperature for ˜2 days prior to vacuum filtration to collect the solid product. The isolated solids were vacuum dried for ˜1 day (574 mg, 90%).

DSC (10° C./min.): weak broad endotherm between ˜130° C. and ˜167° C. possibly associated with the release of solvent. Sharp strong endotherm at ˜190.0° C., (peak maximum) likely associated with the material melting. Elemental analysis—theoretical (for 1:1 salt): 62.58% C, 7.00% H; 6.08% N, 6.08% S. found: 60.41% C, 6.83% H; 5.70% N, 6.85% S. Table 21 summarizes prominent XRPD signals for (+)-doxapram sulfate.

TABLE 21
Summary of XRPD signals for (+)-doxapram sulfate

| 2θ | d space (Å) | Intensity (%) |
|---|---|---|
| 7.97 ± 0.20 | 11.089 ± 0.285 | 17 |
| 8.56 ± 0.20 | 10.333 ± 0.247 | 86 |
| 10.40 ± 0.20 | 8.509 ± 0.166 | 70 |
| 11.00 ± 0.20 | 8.045 ± 0.149 | 27 |
| 12.48 ± 0.20 | 7.090 ± 0.115 | 13 |
| 15.69 ± 0.20 | 5.647 ± 0.072 | 35 |
| 17.16 ± 0.20 | 5.166 ± 0.060 | 35 |
| 17.98 ± 0.20 | 4.933 ± 0.055 | 16 |
| 18.67 ± 0.20 | 4.753 ± 0.051 | 25 |
| 19.69 ± 0.20 | 4.509 ± 0.046 | 39 |
| 20.17 ± 0.20 | 4.402 ± 0.044 | 100 |
| 20.89 ± 0.20 | 4.252 ± 0.041 | 24 |
| 21.12 ± 0.20 | 4.206 ± 0.040 | 25 |
| 21.66 ± 0.20 | 4.103 ± 0.038 | 23 |
| 22.23 ± 0.20 | 3.999 ± 0.036 | 34 |
| 23.21 ± 0.20 | 3.832 ± 0.033 | 28 |
| 23.53 ± 0.20 | 3.781 ± 0.032 | 17 |
| 24.15 ± 0.20 | 3.685 ± 0.030 | 19 |
| 24.62 ± 0.20 | 3.616 ± 0.029 | 15 |
| 25.94 ± 0.20 | 3.435 ± 0.026 | 16 |
| 27.09 ± 0.20 | 3.292 ± 0.024 | 36 |

### Maleate Salt (30)

(+)-Doxapram free base (9, 300 mg, 0.793 mmole) and maleic acid (93.3 mg, 0.803 mmole) were slurried in isopropyl ether (12 mL) at ˜60° C. Methanol (480 μL) was added to this slurry at ˜60° C., which resulted in partial dissolution of the solids followed by precipitation. This reaction mixture was stirred at ˜60° C. for ˜1 day and then allowed to cool naturally to ambient temperature. This ambient temperature mixture was vacuum filtered and solids were isolated (isopropyl ether:methanol ratio was 25:1) (368 mg, 94%).

DSC (10° C./min.): sharp strong endotherm at ˜171.1° C. (peak maximum) likely attributed to the material melting. Elemental analysis—theoretical (for 1:1 salt): 66.65% C, 6.88% H, 5.98% N. found: 67.64% C, 7.12% H; 5.54% N. Table 22 summarizes prominent XRPD signals for (+)-doxapram maleate.

TABLE 22
Summary of XRPD signals for (+)-doxapram maleate

| 2θ | d space (Å) | Intensity (%) |
|---|---|---|
| (5.88-5.93) ± 0.20 | 15.021 ± 0.528-14.894 ± 0.519 | 18-20 |
| (9.56-9.58) ± 0.20 | 9.251 ± 0.197-9.235 ± 0.197 | 23-30 |
| (12.43-12.57) ± 0.20 | 7.118 ± 0.116-7.043 ± 0.113 | 50-60 |
| (13.45-13.49) ± 0.20 | 6.581 ± 0.099-6.565 ± 0.098 | 38-44 |
| (15.23-15.29) ± 0.20 | 5.819 ± 0.077-5.794 ± 0.076 | 49-66 |
| (19.24-19.25) ± 0.20 | 4.614 ± 0.048-4.610 ± 0.048 | 57-74 |
| (19.60-1979) ± 0.20 | 4.528 ± 0.046-4.487 ± 0.045 | 45-53 |
| (20.06-20.07) ± 0.20 | 4.428 ± 0.044-4.424 ± 0.044 | 100 |
| (20.52-20.54) ± 0.20 | 4.328 ± 0.042-4.324 ± 0.042 | 24-30 |
| (21.21-21.29) ± 0.20 | 4.189 ± 0.039-4.173 ± 0.039 | 22-23 |
| (22.65-22.66) ± 0.20 | 3.927 ± 0.035-3.924 ± 0.034 | 21-22 |
| (23.00-23.05) ± 0.20 | 3.868 ± 0.033-3.859 ± 0.033 | 30-32 |

### Oxalate Salt (31)

Doxapram free base (9, 70 mg, 0.185 mmole) and 17 mg of oxalic acid (17 mg, 0,188 mmole) were slurried in isopropyl ether (2.5 mL) at ˜60° C., Methanol (100 μL) was added to this reaction mixture in an attempt to dissolve solids. The resulting reaction mixture was slurried at ˜60° C. for ˜1 day prior to slowly cooling to ambient temperature. This ambient temperature mixture was vacuum filtered to collect the solid product (isopropyl ether:methanol ratio was 25:1). Elemental analysis theoretical (for 1:1 salt): 66.65% C, 6.88% H, 5.98% N. found: 66.47% C, 7.17% H, 5.85% N. Table 23 summarizes prominent XRPD signals for (+)-doxapram oxalate.

TABLE 23
Summary of XRPD signals for (+)-doxapram oxalate

| 2θ | d space (Å) | Intensity (%) |
|---|---|---|
| 8.64 ± 0.20 | 10.233 ± 0.242 | 46 |
| 9.93 ± 0.20 | 8.909 ± 0.183 | 100 |
| 11.03 ± 0.20 | 8.021 ± 0.148 | 15 |
| 12.57 ± 0.20 | 7.043 ± 0.113 | 15 |
| 15.89 ± 0.20 | 5.576 ± 0.071 | 66 |
| 17.33 ± 0.20 | 5.117 ± 0.059 | 11 |
| 17.62 ± 0.20 | 5.035 ± 0.057 | 26 |
| 17.92 ± 0.20 | 4.951 ± 0.055 | 16 |
| 18.30 ± 0.20 | 4.848 ± 0.053 | 16 |
| 18.72 ± 0.20 | 4.741 ± 0.051 | 21 |
| 19.62 ± 0.20 | 4.525 ± 0.046 | 24 |
| 19.87 ± 0.20 | 4.468 ± 0.045 | 72 |
| 20.94 ± 0.20 | 4.242 ± 0.040 | 28 |
| 23.20 ± 0.20 | 3.835 ± 0.033 | 28 |
| 23.53 ± 0.20 | 3.781 ± 0.032 | 29 |
| 24.00 ± 0.20 | 3.708 ± 0.031 | 26 |

### L-tartrate Salt (32)

(+) Doxapram free base (9, 307 mg, 0.811 mmole) and L-tartaric acid (123 mg, 0.820 mmole) were slurried in isopropyl ether (12 mL) at ˜60° C. Methanol (1.5 mL) was added to this reaction mixture in an attempt to dissolve solids. The resulting reaction mixture was slurried at ˜60° C. for ˜1 day and then allowed to naturally cool to ambient temperature prior to slurring at ambient temperature for ˜1 day. Solids were isolated by vacuum filtration. The isolated solids were vacuum dried for ˜1 day (69 mg, 16% as hemisolvate) (isopropyl ether:methanol ratio was 8:1).

Drying data and water resorption after drying support the identity of salt as a variable hydrate. TGA: ˜2.6 wt % loss between ˜26° C. and ˜101° C., attributed to release of methanol and water. DSC (10° C./min.): broad endotherm between ˜0° C. and ˜103° C. (peak maximum at ˜59.2° C.), Sharp broad endotherm at ˜123.5° C. (peak maximum), likely associated with the material melting. Elemental analysis—theoretical (for 1:1 monohydrated salt): 61.52% C, 7.00% H, 5.12% N. found: 61.20% C; 7.11% H, 4.91% N. Table 24 summarizes prominent XRPD signals for (f)-doxapram tartrate.

TABLE 24
Summary of XRPD signals for (+)-doxapram tartrate

| 2θ | d space (Å) | Intensity (%) |
|---|---|---|
| (5.90-5.95) ± 0.20 | 14.978 ± 0.525-14.852 ± 0.516 | 11-12 |
| (8.49-8.54) ± 0.20 | 10.414 ± 0.251-10.353 ± 0.248 | 23-27 |
| (10.03-10.10) ± 0.20 | 8.820 ± 0.179-8.762 ± 0.177 | 34-47 |
| (10.68-10.70) ± 0.20 | 8.284 ± 0.158-8.271 ± 0.157 | 27-30 |
| (12.72-12.80) ± 0.20 | 6.960 ± 0.111-6.915 ± 0.109 | 21-29 |
| (14.67-14.77) ± 0.20 | 6.037 ± 0.083-5.996 ± 0.082 | 19-22 |
| (15.04-15.13) ± 0.20 | 5.890 ± 0.079-5.858 ± 0.078 | 23-29 |
| (18.43-18.57) ± 0.20 | 4.813 ± 0.052-4.779 ± 0.052 | 26-29 |
| (20.09-20.17) ± 0.20 | 4.420 ± 0.044-4.402 ± 0.044 | 100 |
| (20.67-20.79) ± 0.20 | 4.297 ± 0.042-4.273 ± 0.041 | 29-32 |

### Phosphate Salt, Form A (33)

(+) Doxapram free base (9, 502 mg, 1.32 mmole) was slurried in isopropyl ether (18.5 mL) at ambient temperature. To this slurry, a methanolic solution of phosphoric acid (91 μL, 1.34 mmole concentrated phosphoric acid in 735 μL methanol) at ambient temperature was added. The resulting precipitated solids were stirred at ambient temperature for ˜30 minute prior to stirring at ˜60° C. for ˜3 hours. The sample was allowed to slowly cool to ambient temperature and then stirred at sub-ambient (˜2-8° C.) temperature for ˜1 day. The solids were isolated by vacuum filtration (isopropyl ether:methanol ratio was 25:1) (144 mg, 22% yield as monohydrate).

TGA data supports isolation of monohydrate: ˜4.0 wt % loss between ˜29.7° C. and ˜86.4° C. associated with the release of approximately one mole of water per mole of salt. DSC: (10° C./min.): broad endotherm between ˜41° C. and ˜132° C. likely due to loss of water and possibly the dissolution of solids into the fluid (hotstage (microscopy observation). Elemental analysis—theoretical (for 1:1 monohydrate 58.29% C, 7.13% H, 5.66% N, 6.26% P. found: 57.85% C, 7.31% H, 5.23% N, 6.10% P. Table 25 summarizes prominent XRPD signals for (+)-doxapram phosphate, Form A.

TABLE 25
Summary of XRPD signals for (+)-doxapram phosphate, Form A

| 2θ | d space (Å) | Intensity (%) |
|---|---|---|
| 9.56 ± 0.20 | 9.251 ± 0.197 | 61 |
| 10.76 ± 0.20 | 8.220 ± 0.155 | 60 |
| 11.83 ± 0.20 | 7.479 ± 0.128 | 46 |
| 13.47 ± 0.20 | 6.573 ± 0.099 | 73 |
| 13.92 ± 0.20 | 6.361 ± 0.092 | 73 |
| 15.33 ± 0.20 | 5.782 ± 0.076 | 36 |
| 17.57 ± 0.20 | 5.049 ± 0.058 | 65 |
| 18.74 ± 0.20 | 4.736 ± 0.051 | 41 |
| 19.17 ± 0.20 | 4.630 ± 0.048 | 100 |
| 20.07 ± 0.20 | 4.424 ± 0.044 | 83 |
| 21.28 ± 0.20 | 4.176 ± 0.039 | 34 |
| 21.66 ± 0.20 | 4.103 ± 0.038 | 58 |
| 22.16 ± 0.20 | 4.011 ± 0.036 | 43 |
| 22.46 ± 0.20 | 3.958 ± 0.035 | 30 |
| 28.13 ± 0.20 | 3.173 ± 0.022 | 37 |

### Phosphate Salt, Hemihydrate B (34)

(+)Doxapram free base (508 mg, 1.34 mmole) was slurried in isopropyl ether (18.5 ml) at ambient temperature. To this slurry, a methanolic solution of phosphoric acid (92 μL, 1.36 mmole) concentrated phosphoric acid in 735 μL methanol) at ambient temperature was added. The resulting precipitated solids were stirred at ambient temperature for ˜30 minute prior to stirring at ˜60° C. for ˜3 h. The sample was allowed to slowly cool to ambient temperature and then stirred at sub-ambient (˜2-8° C.) temperature for ˜1 day. The solids were isolated by vacuum filtration prior to vacuum drying at ambient temperature for ˜1 day (isopropyl ether:methanol ratio was 25:1) (525 mg, 81% yield as hemihydrate).

TGA supports isolation of hemihydrate: ˜2.3 wt % loss between ˜35° C. and ˜100° C. for loss of ca. 0.6 moles water per moler of salt. Elemental analysis—theoretical (for hemihydrated salt): 59.37% C, 6.97% H, 5.77% N, 6.38% P. found: 58.77% 7.08% H, 5.60% N. Table 26 summarizes prominent XRPD signals for (+)-doxapram phosphate, Form B.

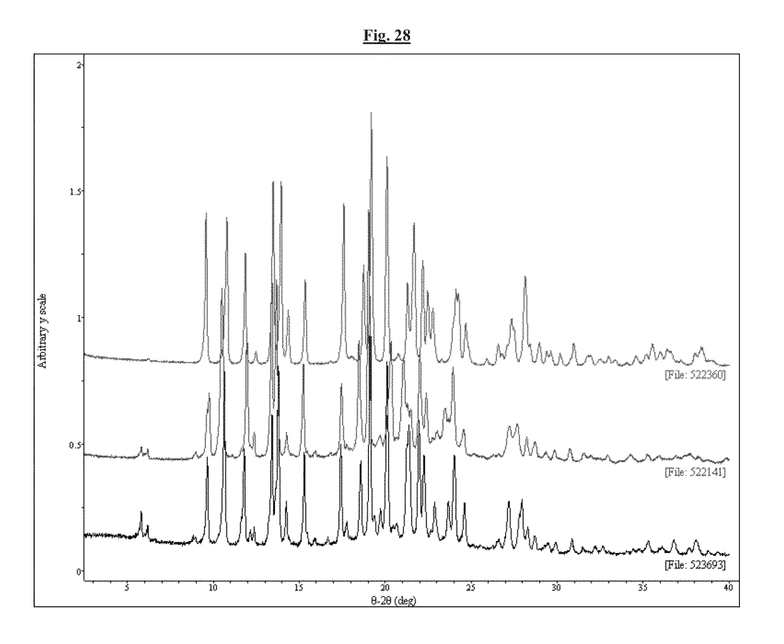

Form B was stored over phosphorus pentoxide. The resulting solid upon TGA analysis showed ˜1.7 wt % loss. The XRPD is similar to but shifted relative to Form B, and is designated (+)-doxapram phosphate, Form C. FIG. 28 shows an overlay of the XRPD diffractograms of (+)-doxapram phosphate Forms A, B, and C.

TABLE 26
Summary of XRPD signals for (+)-doxapram phosphate, Form B

| 2θ | d space (Å) | Intensity (%) |
|---|---|---|
| 9.63 ± 0.20 | 9.187 ± 0.194 | 24 |
| 9.73 ± 0.20 | 9.093 ± 0.190 | 30 |
| 10.48 ± 0.20 | 8.442 ± 0.164 | 70 |
| 11.95 ± 0.20 | 7.406 ± 0.126 | 53 |
| 13.37 ± 0.20 | 6.622 ± 0.100 | 78 |
| 13.65 ± 0.20 | 6.485 ± 0.096 | 73 |
| 15.21 ± 0.20 | 5.826 ± 0.077 | 41 |
| 17.43 ± 0.20 | 5.088 ± 0.059 | 34 |
| 18.45 ± 0.20 | 4.809 ± 0.052 | 50 |
| 19.02 ± 0.20 | 4.666 ± 0.049 | 100 |
| 20.31 ± 0.20 | 4.373 ± 0.043 | 49 |
| 21.04 ± 0.20 | 4.222 ± 0.040 | 44 |
| 21.99 ± 0.20 | 4.042 ± 0.037 | 46 |
| 22.38 ± 0.20 | 3.973 ± 0.035 | 30 |
| 23.46 ± 0.20 | 3.791 ± 0.032 | 25 |
| 23.93 ± 0.20 | 3.718 ± 0.031 | 40 |

### Hydrochloride Salt:

TABLE 27
Summary of XRPD signals for (+)-doxapram hydrochloride

| 2θ | d space (Å) | Intensity (%) |
|---|---|---|
| 8.47 ± 0.20 | 10.427 | 3.0 |
| 10.12 ± 0.20 | 8.735 | 83.0 |
| 11.39 ± 0.20 | 7.760 | 21.9 |
| 11.96 ± 0.20 | 7.394 | 5.0 |
| 12.67 ± 0.20 | 6.979 | 19.6 |
| 14.68 ± 0.20 | 6.030 | 1.4 |
| 15.87 ± 0.20 | 5.679 | 100.0 |
| 16.88 ± 0.20 | 5.248 | 15.4 |
| 17.43 ± 0.20 | 5.082 | 43.5 |
| 17.84 ± 0.20 | 4.967 | 22.5 |
| 18.40 ± 0.20 | 4.817 | 46.3 |
| 19.24 ± 0.20 | 4.610 | 33.9 |
| 19.76 ± 0.20 | 4.489 | 22.3 |
| 20.72 ± 0.20 | 4.284 | 89.2 |
| 21.68 ± 0.20 | 4.096 | 34.5 |
| 22.84 ± 0.20 | 3.890 | 38.7 |
| 23.40 ± 0.20 | 3.799 | 38.1 |
| 24.35 ± 0.20 | 3.652 | 59.3 |
| 25.44 ± 0.20 | 3.499 | 6.2 |
| 26.24 ± 0.20 | 3.394 | 22.4 |
| 27.07 ± 0.20 | 3.291 | 6.4 |
| 28.13 ± 0.20 | 3.170 | 10.9 |
| 29.16 ± 0.20 | 3.060 | 36.7 |
| 29.89 ± 0.20 | 2.988 | 45.5 |
| 30.83 ± 0.20 | 2.898 | 4.8 |
| 31.20 ± 0.20 | 2.864 | 10.3 |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising at least one crystalline salt of (R)-doxapram selected from the group consisting of
(i) a crystalline sulfate salt of (R)-doxapram, wherein the XRPD spectrum of the salt comprises 2θ values (in°) of 7.97±0.20, 8.56±0.20, 10.40±0.20, 11.00±0.20, 12.48±0.20, 15.69±0.20, 17.16±0.20, 17.98±0.20, 18.67±0.20, 19.69±0.20, 20.17±0.20, 20.89±0.20, 21.12±0.20, 21.66±0.20, 22.23±0.20, 23.21±0.20, 23.53±0.20, 24.15±0.20, 24.62±0.20, 25.94±0.20, and 27.09±0.20;
(ii) a crystalline 1)-glucuronate salt of (R)-doxapram, wherein the XRPD spectrum of the salt comprises 2θ values (in°) of 7.64±0.20, 8.69±0.20, 9.76±0.20, 11.63±0.20, 12.33±0.20, 13.34±0.20, 15.33±0.20, 15.98±0.20, 16.96±0.20, 17.43±0.20, 18.08±0.20, 18.55±0.20, 19.54±0.20, 20.21±0.20, 20.61±0.20, 20.91±0.20, 21.56±0.20, 22.73±0.20, 23.35±0.20, 24.82±0.20, and 25.37±0.20;
(iii) a crystalline maleate salt of (R)-doxapram, wherein the XRPD spectrum of the salt comprises 2θ values (in°) of (5.88-5.93)±0.20, (9.56-9.58)±0.20, (12.43-12.57)±0.20, (13.45-13.49)±0.20, (15.23-15.29)±0.20, (19.24-19.25)±0.20, (19.60-19.79)±0.20, (20.06-20.07)±0.20, (20.52-20.54)±0.20, (21.21-21.29)±0.20, (22.65-22.66)±0.20, and (23.00-23.05)±0.20;
(iv) a crystalline oxalate salt of (R)-doxapram, wherein the XRPD spectrum of the salt comprises 2θ values (in°) of 8.64±0.20, 9.93±0.20, 11.03±0.20, 15.89±0.20, 17.33±0.20, 17.62±0.20, 17.92±0.20, 18.30±0.20, 18.72±0.20, 19.62±0.20, 19.87±0.20, 20.94±0.20, 23.20±0.20, 23.53±0.20, and 24.00±0.20;
(v) a crystalline L-tartrate salt of (R)-doxapram, wherein the XRPD spectrum of the salt comprises 2θ values (in°) of (5.90-5.95)±0.20, (8.49-8.54)±0.20, (10.03-10.10)±0.20, (10.68-10.70)±0.20, (12.72-12.80)±0.20, (14.67-14.77)±0.20, (15.04-15.13)±0.20, (18.43-18.57)±0.20, (20.09-20.17)±0.20, and (20.67-20.79)±0.20;
(vi) a crystalline phosphate salt of (R)-doxapram, wherein the XRPD spectrum of the salt comprises 2θ values (in c) of 9.56±0.20, 10.76±0.20, 11.83±0.20, 13.47±0.20, 13.92±0.20, 15.33±0.20, 17.57±0.20, 18.74±0.20, 19.17±0.20, 20.07±0.20, 21.28±0.20, 22.16±0.20, 21.66±0.20, 22.46±0.20, and 28.13±0.20;
(vii) a crystalline phosphate salt of (R)-doxapram, wherein the XRPD spectrum of the salt comprises 2θ values (in c) of 9.63±0.20, 9.73±0.20, 10.48±0.20, 11.95±0.20, 11.95±0.20, 13.37±0.20, 13.65±0.20, 15.21±0.20, 17.43±0.20, 18.45±0.20, 19.02±0.20, 20.31±0.20, 21.04±0.20, 21.99±0.20, 22.38±0.20, 23.46±0.20, and 23.93±0.20;
(viii) a crystalline hydrochloride salt of (R)-doxapram, wherein the XRPD spectrum of the salt comprises 2θ values (in°) of 7.47±0.20, 10.12±0.20, 11.39±0.20, 11.96±0.20, 12.64±0.20, 14.68±0.20, 15.87±0.20, 16.88±0.20, 17.44±0.20, 17.83±0.20, 18.40±0.20, 10.24±0.20, 19.76±0.20, 20.72±0.20, 21.68±0.20, 22.8.4±0.20, 23.40±0.20, 24.36±0.20, 25.44±0.20, 26.24±0.20, 27.07±0.20, 28.13±0.20, 29.16±0.20, 29.88±0.20, 30.83, 3120±0.20;
and any combinations thereof.

2. The salt of claim 1, wherein the salt in (i) comprises about one molar equivalent of sulfuric acid.

3. The salt of claim 1, wherein the salt in (ii) comprises about one molar equivalent of D-glucuronic acid.

4. The salt of claim 1, wherein the salt in (iii) comprises about one molar equivalent of maleic acid.

5. The salt of claim 1, wherein the salt in (iv) comprises about one molar equivalent of oxalic acid.

6. The salt of claim 1, wherein the salt in (v) comprises about one molar equivalent of L-tartaric acid and about 0.4 to 0.6 molar equivalents of methanol.

7. The salt of claim 1, wherein the salt in (vi) comprises about one molar equivalent of phosphoric acid and about one molar equivalent of water.

8. The salt of claim 1, wherein the salt in (vii) comprises about one molar equivalent of phosphoric acid and about 0.6 molar equivalents of water.

9. The salt of claim 1 wherein the salt in (viii) comprises one molar equivalent of hydrochloric acid and about one molar equivalent of water.

10. A composition comprising at least one compound selected from the group consisting of
(R)-2-(1-Ethylpyrrolidin-3-yl-2,2-diphenylacetonitrile;
(R)-2-(1-Ethylpyrrolidin-3-yl)-2,2-diphenylacetamide;
(R)-2-(1-Ethylpyrrolidin-3-yl)-2,2-diphenylacetic acid;
(R)-1-Ethyl-4-(2-Iodo-ethyl)-3,3-diphenyl-pyrrolidin-2-one;
(R)-1-Ethyl-4-(2-d₈-morpholin-4-yl-ethyl)-3,3-diphenyl-pyrrolidin-2-one;
a salt thereof, and any combinations thereof.

11. A method of preparing a composition comprising doxapram or a salt thereof, comprising the steps of:
(i) cyclizing an ester derivative of (Z)-4-diphenylacetyl-ethyl-amino)-but-2-en-1-ol to generate 1-ethyl-3,3-diphenyl-4-vinyl-pyrrolin-2-one;
(ii) derivatizing 1-ethyl-3,3-diphenyl-4-vinyl-pyrrolin-2-one to generate 4-(2-X-ethyl)-1-ethyl-3,3-diphenyl-pyrrolidin-2-one, wherein X is a leaving group; and
(iii) reacting 4-(2-X-ethyl)-1-ethyl-3,3-diphenyl-pyrrolidin-2-one with morpholine to generate doxapram or a salt thereof.

12. The method of claim 11, wherein the ester derivative in (i) is acetate.

13. The method of claim 11, wherein X in (ii) is selected from the group consisting of fluoride, chloride, bromide, triflate, tosylate and mesylate.

14. The method of claim 11, wherein the cyclizing in (i) comprises reacting the ester derivative of (Z)-4-diphenylacetyl-ethyl-amino)-but-2-en-1-ol with a strong base.

15. The method of claim 14, wherein the strong base comprises potassium hexamethyldisilazide, sodium hexamethyldisilazide or sparteine.

16. The method of claim 11, wherein the cyclizing in (i) comprises the steps of:
derivatizing the ester derivative of (Z)-4-diphenylacetyl-ethyl-amino)-but-2-en-1-ol to generate a carbonate derivative of (Z)-4-diphenylacetyl-ethyl-amino)-but-2-en-1-ol; and,
reacting the carbonate derivative of (Z)-4-diphenylacetyl-ethyl-amino)-but-2-en-1-ol with a strong base to generate 1-ethyl-3,3-diphenyl-4-vinyl-pyrrolin-2-one.

17. The method of claim 16, wherein the strong base comprises potassium hexamethyldisilazide, sodium hexamethyldisilazide or sparteine.

18. The method of claim 11, wherein the doxapram or a salt thereof is enantiomerically enriched in (R)-doxapram.

19. The method of claim 18, wherein (R)-doxapram or a salt thereof is in at least about 70% enantiomeric excess in the composition.

20. The method of claim 18, wherein the cyclizing in (i) comprises the steps of:
hydrolyzing the ester derivative of (Z)-4-diphenylacetyl-ethyl-amino)-but-2-en-1-ol to generate (Z)-4-diphenylacetyl-ethyl-amino)-but-2-en-1-ol;
derivatizing (Z)-4-diphenylacetyl-ethyl-amino)-but-2-en-1-ol to generate bis(η³-(ethyl-(diphenylacetyl)aminomethylyl)-allyl)-dichloro-dipalladium(II); and,
reacting bis(η³-1-(ethyl-(diphenylacetyl)aminomethylyl)-allyl)-dichloro-dipalladium(II) with R-BINAP in the presence of a strong base to generate 1-ethyl-3,3-diphenyl-4-vinyl-pyrrolin-2-one enriched in the (R)-enantiomer.

21. A method of preparing a composition comprising (R)-doxapram, wherein in the composition (R)-doxapram is in an enantiomeric excess over (S)-doxapram, the method comprising the steps of
(i) hydrolyzing (R)-2-(1-ethylpyrrolidin-3-yl)-2,2-diphenylacetic acid to generate (R)-2-(1-ethylpyrrolidin-3-yl)-2,2-diphenylacetic acid;
(ii) derivatizing (R)-2-(1-ethylpyrrolidin-3-yl)-2,2-diphenylacetic acid to generate (R)-1-ethyl-4-(2-iodoethyl)-3,3-diphenylpyrrolidin-2-one; and
(iii) reacting (R)-1-ethyl-4-(2-iodoethyl)-3,3-diphenylpyrrolidin-2-one with morpholine to generate (R)-doxapram.

22. The method of claim 21, wherein the hydrolyzing in comprises the steps of:
hydrolyzing (R)-2-(1-ethylpyrrolidin-3-yl)-2,2-diphenylacetonitrile with acid generate (R)-2-(1-ethylpyrrolidin-3-yl)-2,2-diphenylacetamide; and
hydrolyzing (R)-2-(1-ethylpyrrolidin-3-yl)-2,2-diphenylacetamide with acid to generate (R)-2-(1-ethylpyrrolidin-3-yl)-2,2-diphenylacetic acid.

23. The method of claim 21, wherein the derivatizing in (ii) comprises contacting (R)-2-(1-ethylpyrrolidin-3-yl)-2,2-diphenylacetic acid with acetic anhydride and an iodide salt.

24. The method of claim 21, wherein the (R)-2-(1-ethylpyrrolidin-3-yl)-2,2-diphenylacetonitrile to be used in (i) is of at least about 90% enantiomeric excess.

25. The method of claim 24, wherein the (R)-2-(1-ethylpyrrolidin-3-yl)-2,2-diphenylacetonitrile to be used in (i) is of at least about 95% enantiomeric excess.

26. The method of claim 25, wherein the (R)-2-(1-ethylpyrrolidin-3-yl)-2,2-diphenylacetonitrile to be used in (1) is of at least about 98% enantiomeric excess.

27. The method of claim 26, wherein the (R)-2-(1-ethylpyrrolidin-3-yl)-2,2-diphenylacetonitrile to be used in (i) is of at least about 99% enantiomeric excess.

28. The method of claim 21, wherein the (R)-2-(1-ethylpyrrolidin-3-yl)-2,2-diphenylacetonitrile to be used in (i) is chirally enriched by diastereoselective precipitation of its salt with a chiral organic acid.

29. The method of claim 28, wherein the chiral organic acid comprises (+)-dibenzoyl-D-tartaric acid (D-DBTA).

30. The method of claim 21, wherein (R)-2-(1-ethylpyrrolidin-3-yl)-2,2-diphenylacetonitrile is generated by contacting (R)-3-X-1-ethylpyrrolidine with a reaction mixture comprising diphenylacetonitrile and a strong base, wherein X is a leaving group.

31. The method of claim 30, wherein X is selected from the group consisting of chloride, bromide, iodide, tosylate, mesylate and triflate.

32. The method of claim 31, wherein X is chloride and the reaction mixture further comprises a promoter.

33. The method of claim 32, wherein the promoter comprises an iodide salt and the contacting is performed at a temperature of about 50° C. to about 145° C.

34. The method of claim 32, wherein (R)-3-chloride-1-ethylpyrrolidine is generated from (S)-1-ethylpyrrolidin-3-ol.

35. The method of claim 34, wherein (S)-1-ethylpyrrolidin-3-ol is reacted with thionyl chloride to generate (R)-3-chloride-1-ethylpyrrolidine.

36. The method of claim 30, wherein X is tosylate or mesylate.

37. The method of claim 36, wherein the contacting is performed at a temperature of about 50° C. to about 145° C.

38. The method of claim 36, wherein (R)-3-X-1-ethylpyrrolidine is generated by reacting (R)-1-ethyl-3-pyrrolidinol with tosyl chloride or mesyl chloride.

39. The method of claim 38, wherein (R)-3-X-1-ethylpyrrolidine is not isolated before being derivatized to generate (R)-2-(1-ethylpyrrolidin-3-yl)-2,2-diphenylacetonitrile.
